SEQUENCE LISTING (86 sequences)

SEQ ID NO: 1 - protein, 44 aa, Homo sapiens
TTTPAPRPPTPAPTIASQPLSLRPEACRPAAGGAVHTRGLDFAC

SEQ ID NO: 2 - DNA, 132 bp, Homo sapiens
  source 1..132: mol_type = genomic DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgt

SEQ ID NO: 3 - protein, 30 aa, Homo sapiens
QTTPGERSSLPAFYPGTSGSCSGCGSLSLP

SEQ ID NO: 4 - DNA, 90 bp, Homo sapiens
  source 1..90: mol_type = genomic DNA
cagacaacaccaggcgagagatctagcctgcccgccttctaccctggcaccagcggctcttgttctggctgtggcagcctgtctctgccc

SEQ ID NO: 5 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 25 aa, Homo sapiens
DIYIWAPLAGTCGVLLLSLVITLYC

SEQ ID NO: 7 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 23 aa, Homo sapiens
LGLLVAGVLVLLVSLGVAIHLCC

SEQ ID NO: 9 - protein, 26 aa, Homo sapiens
VAAILGLGLVLGLLGPLAILLALYLL

SEQ ID NO: 10 - protein, 25 aa, Homo sapiens
ALIVLGGVAGLLLFIGLGIFFCVRC

SEQ ID NO: 11 - protein, 23 aa, Homo sapiens
LCYLLDGILFIYGVILTALFLRV

SEQ ID NO: 12 - protein, 26 aa, Homo sapiens
WVLVVVGGVLACYSLLVTVAFIIFWV

SEQ ID NO: 13 - protein, 24 aa, Homo sapiens
IYIWAPLAGTCGVLLLSLVITLYC

SEQ ID NO: 14 - protein, 24 aa, Homo sapiens
ALPAALAVISFLLGLGLGVACVLA

SEQ ID NO: 15 - DNA, 72 bp, Homo sapiens
  source 1..72: mol_type = genomic DNA
atctatatttgggcacccctggctggaacctgcggagtgctgctgctgtctctcgtgattacactgtattgc

SEQ ID NO: 16 - DNA, 75 bp, Homo sapiens
  source 1..75: mol_type = genomic DNA
gatatctacatctgggcgcccttggccgggacttgtggggtccttctcctgtcactggttatcaccctttactgc

SEQ ID NO: 17 - DNA, 321 bp, synthetic construct
  source 1..321: mol_type = other DNA
gacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaca

SEQ ID NO: 18 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 19 - DNA, 45 bp, synthetic construct
  source 1..45: mol_type = other DNA
ggcggcggaggatctggcggaggcggaagtggcggagggggatct

SEQ ID NO: 20 - DNA, 360 bp, synthetic construct
  source 1..360: mol_type = other DNA
gaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagc

SEQ ID NO: 21 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 22 - protein, 10 aa, synthetic construct
GSGSGSGSGS

SEQ ID NO: 23 - protein, 8 aa, synthetic construct
GSGSGSGS

SEQ ID NO: 24 - protein, 8 aa, synthetic construct
RSGSGSGS

SEQ ID NO: 25 - DNA, 30 bp, synthetic construct
  source 1..30: mol_type = other DNA
ggatccggcagcggatctggcagtggaagc

SEQ ID NO: 26 - DNA, 24 bp, synthetic construct
  source 1..24: mol_type = other DNA
ggatctggctctggaagcggcagc

SEQ ID NO: 27 - DNA, 24 bp, synthetic construct
  source 1..24: mol_type = other DNA
agatccggatctggaagtggctcc

SEQ ID NO: 28 - DNA, 24 bp, synthetic construct
  source 1..24: mol_type = other DNA
ggaagtggatctgggagcggctct

SEQ ID NO: 29 - DNA, 24 bp, synthetic construct
  source 1..24: mol_type = other DNA
ggatcaggcagtggctctggcagc

SEQ ID NO: 30 - DNA, 18 bp, synthetic construct
  source 1..18: mol_type = other DNA
ggatctggaagtggctcc

SEQ ID NO: 31 - protein, 15 aa, synthetic construct
GGGGSGGGGSGGGGS

SEQ ID NO: 32 - protein, 509 aa, Homo sapiens
MGCGCSSHPEDDWMENIDVCENCHYPIVPLDGKGTLLIRNGSEVRDPLVTYEGSNPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 33 - protein, 509 aa, Homo sapiens
MGCVCSSNPEDDWMENIDVCENCHYPIVPLDSKISLPIRNGSEVRDPLVTYEGSLPPASPLQDNLVIALHSYEPSHDGDLGFEKGEQLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHDLVRHYTNASDGLCTKLSRPCQTQKPQKPWWEDEWEVPRETLKLVERLGAGQFGEVWMGYYNGHTKVAVKSLKQGSMSPDAFLAEANLMKQLQHPRLVRLYAVVTQEPIYIITEYMENGSLVDFLKTPSGIKLNVNKLLDMAAQIAEGMAFIEEQNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYHLMMLCWKERPEDRPTFDYLRSVLDDFFTATEGQYQPQP

SEQ ID NO: 34 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 35 - protein, 327 aa, Homo sapiens
MLWIWAVLPLVLAGSQLRVHTQGTNSISESLKLRRRVRETDKNCSEGLYQGGPFCCQPCQPGKKKVEDCKMNGGTPTCAPCTEGKEYMDKNHYADKCRRCTLCDEEHGLEVETNCTLTQNTKCKCKPDFYCDSPGCEHCVRCASCEHGTLEPCTATSNTNCRKQSPRNRLWLLTILVLLIPLVFIYRKYRKRKCWKRRQDDPESRTSSRETIPMNASNLSLSKYIPRIAEDMTIQEAKKFARENNIKEGKIDEIMHDSIQDTAEQKVQLLLCWYQSHGKSDAYQDLIKGLKKAECRRTLDKFQDMVQKDLGKSTPDTGNENEGQCLE

SEQ ID NO: 36 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 37 - protein, 454 aa, Homo sapiens
MGLPTVPGLLLSLVLLALLMGIHPSGVTGLVPSLGDREKRDSLCPQGKYVHSKNNSICCTKCHKGTYLVSDCPSPGRDTVCRECEKGTFTASQNYLRQCLSCKTCRKEMSQVEISPCQADKDTVCGCKENQFQRYLSETHFQCVDCSPCFNGTVTIPCKETQNTVCNCHAGFFLRESECVPCSHCKKNEECMKLCLPPPLANVTNPQDSGTAVLLPLVILLGLCLLSFIFISLMCRYPRWRPEVYSIICRDPVPVKEEKAGKPLTPAPSPAFSPTSGFNPTLGFSTPGFSSPVSSTPISPIFGPSNWHFMPPVSEVVPTQGADPLLYESLCSVPAPTSVQKWEDSAHPQRPDNADLAILYAVVDGVPPARWKEFMRFMGLSEHEIERLEMQNGRCLREAQYSMLEAWRRRTPRHEDTLEVVGLVLSKMNLAGCLENILEALRNPAPSSTTRLPR

SEQ ID NO: 38 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 39 - protein, 474 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRKKKPSCLQRDAKVPHVPDEKSQDAVGLEQQHLLTTAPSSSSSSLESSASAGDRRAPPGGHPQARVMAEAQGFQEARASSRISDSSHGSHGTHVNVTCIVNVCSSSDHSSQCSSQASATVGDPDAKPSASPKDEQVPFSQEECPSQSPCETTETLQSHEKPLPLGVPDMGMKPSQAGWFDQIAVKVA

SEQ ID NO: 40 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 41 - protein, 239 aa, Mus musculus
MESVVQPSVFVVDGQTDIPFRRLEQNHRRRRCGTVQVSLALVLLLGAGLATQGWFLLRLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 42 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 43 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 44 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 45 - protein, 42 aa, Homo sapiens
KRGRKKLLYIFKQPFMRPVQTTQEEDGCSCRFPEEEEGGCEL

SEQ ID NO: 46 - DNA, 126 bp, Homo sapiens
  source 1..126: mol_type = genomic DNA
aaaaggggccggaaaaagctgctgtatattttcaaacagccttttatgaggcctgtgcagacaacacaggaagaggacggctgtagctgtcggttccccgaagaggaagaggggggctgcgaactg

SEQ ID NO: 47 - protein, 41 aa, Homo sapiens
KRGRKKLLYIFKQPFMRPVQTTQEEDGCSCRFPEEEEGGCE

SEQ ID NO: 48 - DNA, 123 bp, Homo sapiens
  source 1..123: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaa

SEQ ID NO: 49 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 50 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 51 - protein, 48 aa, Homo sapiens
QRRKYRSNKGESPVEPAEPCHYSCPREEEGSTIPIQEDYRKPEPACSP

SEQ ID NO: 52 - protein, 250 aa, Mus musculus
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRPLCSSDCIRIFVTFSSMFLIFVLGAILFFHQRRNHGPNEDRQAVPEEPCPYSCPREEEGSAIPIQEDYRKPEPAFYP

SEQ ID NO: 53 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 54 - protein, 42 aa, Homo sapiens
ALYLLRRDQRLPPDAHKPPGGGSFRTPIQEEQADAHSTLAKI

SEQ ID NO: 55 - protein, 272 aa, Mus musculus
MYVWVQQPTALLLLALTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 56 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 57 - protein, 344 aa, Mus musculus
MKCKFLGSFFLLFSLSGKGADCRDNETIWGVLGHGITLNIPNFQMTDDIDEVRWVRRGTLVAEFKRKKPPFLISETYEVLANGSLKIKKPMMRNDSGTYNVMVYGTNGMTRLEKDLDVRILERVSKPMIHWECPNTTLTCAVLQGTDFELKLYQGETLLNSLPQKNMSYQWTNLNAPFKCEAINPVSKESKMEVVNCPEKGLSFYVTVGVGAGGLLLVLLVALFIFCICKRRKRNRRRKDEELEIKASRTSTVERGPKPHSTPAAAAQNSVALQAPPPPGHHLQTPGHRPLPPGHRTREHQQKKRPPPSGTQIHQQKGPPLPRPRVQPKPPCGSGDGVSLPPPN

SEQ ID NO: 58 - protein, 495 aa, Homo sapiens
MPMGSLQPLATLYLLGMLVASCLGRLSWYDPDFQARLTRSNSKCQGQLEVYLKDGWHMVCSQSWGRSSKQWEDPSQASKVCQRLNCGVPLSLGPFLVTYTPQSSIICYGQLGSFSNCSHSRNDMCHSLGLTCLEPQKTTPPTTRPPPTTTPEPTAPPRLQLVAQSGGQHCAGVVEFYSGSLGGTISYEAQDKTQDLENFLCNNLQCGSFLKHLPETEAGRAQDPGEPREHQPLPIQWKIQNSSCTSLEHCFRKIKPQKSGRVLALLCSGFQPKVQSRLVGGSSICEGTVEVRQGAQWAALCDSSSARSSLRWEEVCREQQCGSVNSYRVLDAGDPTSRGLFCPHQKLSQCHELWERNSYCKKVFVTCQDPNPAGLAAGTVASIILALVLLVVLLVVCGPLAYKKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 59 - protein, 494 aa, Mus musculus
MDSHEVLLAATYLLGTLAAFCLGQSGRGGLDIQVMLSGSNSKCQGQVEIQMENKWKTVCSSSWRLSQDHSKNAQQASAVCKQLRCGDPLALGPFPSLNRPQNQVFCQGSPWSISNCNNTSSQDQCLPLSLICLEPQRTTPPPTTTPPTTVPEPTAPPRLQLVPGHEGLRCTGVVEFYNGSWGGTILYKAKDRPLGLGNLICKSLQCGSFLTHLSGTEAAGTPAPAELRDPRPLPIRWEAPNGSCVSLQQCFQKTTAQEGGQALTVICSDFQPKVQSRLVGGSSVCEGIAEVRQRSQWEALCDSSAARGRGRWEELCREQQCGDLISFHTVDADKTSPGFLCAQEKLSQCYHLQKKKHCNKRVFVTCQDPNPAGLAPGTVASIILTLVLLVVLLAMCGPLVYKKLVKKFRQKKQRQWIGPTGVNQNMSFHRSHTATVRSQVENPTASHVDNEYSQPPRNSHLSAYPALEGALHRSSTQPDNSSDSDYDLQVAQRL

SEQ ID NO: 60 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 61 - protein, 498 aa, Mus musculus
MSALLTAAGLLFLGMLQAFPTDRPLKTTCAGDLSHYPGEAARNCCYQCPSGLSPTQPCPRGPAHCRKQCAPDYYVNEDGKCTACVTCLPGLVEKAPCSGNSPRICECQPGMHCCTPAVNSCARCKLHCSGEEVVKSPGTAKKDTICELPSSGSGPNCSNPGDRKTLTSHATPQAMPTLESPANDSARSLLPMRVTNLVQEDATELVKVPESSSSKAREPSPDPGNAEKNMTLELPSPGTLPDISTSENSKEPASTASTLSLVVDAWTSSRMQPTSPLSTGTPFLDPGPVLFWVAMVVLLVGSGSFLLCYWKACRRRFQQKFHLDYLVQTFQPKMEQTDSCPTEKLTQPQRSGSVTDPSTGHKLSPVSPPPAVETCASVGATYLENLPLLDDSPAGNPFSPREPPEPRVSTEHTNNRIEKIYIMKADTVIVGSVKTEVPEGRAPAGSTESELEAELEVDHAPHYPEQETEPPLGSCTEVMFSVEEGGKEDHGPTTVSEK

SEQ ID NO: 62 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 63 - protein, 63 aa, Homo sapiens
KKVAKKPTNKAPHPKQEPQEINFPDDLPGSNTAAPVQETLHGCQPVTQAEDGKESRISVQERQ

SEQ ID NO: 64 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 65 - protein, 41 aa, Homo sapiens
RSKRSRLLHSDYMNMTPRRPGPTRKHYQPYAPPRDFAAYRS

SEQ ID NO: 66 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 67 - protein, 1170 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 68 - protein, 59 aa, Homo sapiens
YKVGFFKRNLKEKMEAGRGVPNGIPAEDSEQLASGQEAGDPGCLKPLHEKDSESGGGKD

SEQ ID NO: 69 - protein, 1162 aa, Mus musculus
MSFRIAGPRLLLLGLQLFAKAWSYNLDTRPTQSFLAQAGRHFGYQVLQIEDGVVVGAPGEGDNTGGLYHCRTSSEFCQPVSLHGSNHTSKYLGMTLATDAAKGSLLACDPGLSRTCDQNTYLSGLCYLFPQSLEGPMLQNRPAYQECMKGKVDLVFLFDGSQSLDRKDFEKILEFMKDVMRKLSNTSYQFAAVQFSTDCRTEFTFLDYVKQNKNPDVLLGSVQPMFLLTNTFRAINYVVAHVFKEESGARPDATKVLVIITDGEASDKGNISAAHDITRYIIGIGKHFVSVQKQKTLHIFASEPVEEFVKILDTFEKLKDLFTDLQRRIYAIEGTNRQDLTSFNMELSSSGISADLSKGHAVVGAVGAKDWAGGFLDLREDLQGATFVGQEPLTSDVRGGYLGYTVAWMTSRSSRPLLAAGAPRYQHVGQVLLFQAPEAGGRWNQTQKIEGTQIGSYFGGELCSVDLDQDGEAELLLIGAPLFFGEQRGGRVFTYQRRQSLFEMVSELQGDPGYPLGRFGAAITALTDINGDRLTDVAVGAPLEEQGAVYIFNGKPGGLSPQPSQRIQGAQVFPGIRWFGRSIHGVKDLGGDRLADVVVGAEGRVVVLSSRPVVDVVTELSFSPEEIPVHEVECSYSAREEQKHGVKLKACFRIKPLTPQFQGRLLANLSYTLQLDGHRMRSRGLFPDGSHELSGNTSITPDKSCLDFHFHFPICIQDLISPINVSLNFSLLEEEGTPRDQKVGRAMQPILRPSIHTVTKEIPFEKNCGEDKKCEANLTLSSPARSGPLRLMSSASLAVEWTLSNSGEDAYWVRLDLDFPRGLSFRKVEMLQPHSRMPVSCEELTEGSSLLTKTLKCNVSSPIFKAGQEVSLQVMFNTLLNSSWEDFVELNGTVHCENENSSLQEDNSAATHIPVLYPVNILTKEQENSTLYISFTPKGPKTQQVQHVYQVRIQPSAYDHNMPTLEALVGVPWPHSEDPITYTWSVQTDPLVTCHSEDLKRPSSEAEQPCLPGVQFRCPIVFRREILIQVTGTVELSKEIKASSTLSLCSSLSVSFNSSKHFHLYGSKASEAQVLVKVDLIHEKEMLHVYVLSGIGGLVLLFLIFLALYKVGFFKRNLKEKMEADGGVPNGSPPEDTDPLAVPGEETKDMGCLEPLRESDKD

SEQ ID NO: 70 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 71 - protein, 537 aa, Mus musculus
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 72 - protein, 231 aa, Homo sapiens
MSKQRGTFSEVSLAQDPKRQQRKPKGNKSSISGTEQEIFQVELNLQNPSLNHQGIDKIYDCQGLLPPPEKLTAEVLGIICIVLMATVLKTIVLIPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 73 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 74 - protein, 58 aa, Homo sapiens
QLGLHIWQLRSQCMWPRETQLLLEVPPSTEDARSCQFPEEERGERSAEEKGRLGDLWV

SEQ ID NO: 75 - protein, 228 aa, Mus musculus
MGAWAMLYGVSMLCVLDLGQPSVVEEPGCGPGKVQNGSGNNTRCCSLYAPGKEDCPKERCICVTPEYHCGDPQCKICKHYPCQPGQRVESQGDIVFGFRCVACAMGTFSAGRDGHCRLWTNCSQFGFLTMFPGNKTHNAVCIPEPLPTEQYGHLTVIFLVMAACIFFLTTVQLGLHIWQLRRQHMCPRETQPFAEVQLSAEDACSFQFPEEERGEQTEEKCHLGGRWP

SEQ ID NO: 76 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 77 - protein, 24 aa, Homo sapiens
LCARPRRSPAQEDGKVYINMPGRG

SEQ ID NO: 78 - protein, 79 aa, Mus musculus
MDPPGYLLFLLLLPVAASQTSAGSCSGCGTLSLPLLAGLVAADAVMSLLIVGVVFVCMRPHGRPAQEDGRVYINMPGRG

SEQ ID NO: 79 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 80 - protein, 52 aa, Homo sapiens
YFLGRLVPRGRGAAEAATRKQRITETESPYQELQGQRSDVYSDLNTQRPYYK

SEQ ID NO: 81 - protein, 114 aa, Mus musculus
MGALEPSWCLLFLPVLLTVGGLSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 82 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 83 - protein, 113 aa, Homo sapiens
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - DNA, 339 bp, Homo sapiens
  source 1..339: mol_type = genomic DNA
ctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 85 - DNA, 17 bp, synthetic construct
  source 1..17: mol_type = other DNA
aaataaaatacgaaatg

SEQ ID NO: 86 - protein, 62 aa, Homo sapiens
KKVAKKPTNKAPHPKQEPQEINFPDDLPGSNTAAPVQETLHGCQPVTQEDGKESRISVQERQ

(19) United States
(12) Patent Application Publication
(10) Pub. No.: US 2026/0275283 A1
(43) Pub. Date: Sep. 17, 2026

(54) SINGLE-AND MULTI-CHAIN CHIMERIC ANTIGEN RECEPTORS

(71) Applicant: Cell Design Labs, Inc., Emeryville, CA (US)

(72) Inventors: Peter Emtage, Lafayette, CA (US); Sarah Wyman, Oakland, CA (US)

(21) Appl. No.: 19/530,577

(22) Filed: Feb. 5, 2026

Related U.S. Application Data
(62) Division of application No. 16/226,158, filed on Dec. 19, 2018, now Pat. No. 12,570,958.
(60) Provisional application No. 62/609,895, filed on Dec. 22, 2017.
(60) Provisional application No. 62/767,212, filed on Nov. 14, 2018.

(51) Int. Cl. C12N 5/0783; A61K 40/11; A61K 40/31; A61K 40/42; A61P 35/00; C07K 14/47; C07K 14/705; C07K 16/28; C07K 16/46; C12N 15/62

(52) U.S. Cl. CPC C12N 5/0636; A61K 40/11; A61K 40/31; A61K 40/4211; A61P 35/00; C07K 14/4748; C07K 14/7051; C07K 14/70521; C07K 16/28; C07K 16/283; C07K 16/2863; C07K 16/46; C12N 15/62; A61K 2239/38; A61K 2239/48; C07K 2319/03; C07K 2319/70

(57) ABSTRACT

Provided herein are single-chain and multi-chain chimeric antigen receptors, nucleic acids encoding the same, and mammalian cells expressing the same. Also provided are methods of treating a cancer in a subject using a mammalian cell expressing any of these single-chain and multi-chain chimeric antigen receptors.

### CROSS-REFERENCE TO RELATED APPLICATIONS

This application is a divisional of U.S. patent application Ser. No. 16/226,158 filed Dec. 19, 2018, which claims priority to U.S. Patent Application No. 62/609,895, filed Dec. 22, 2017, and U.S. Provisional Patent Application No. 62/767,212, filed Nov. 14, 2018, the entire contents of each of these applications are incorporated by reference herein.

### SEQUENCE LISTING

This application contains a Sequence Listing that has been submitted electronically as an XML file named 35468-0390002 SL ST26.xml. The XML file, created on Feb. 2, 2026, is 92,283 bytes in size. The material in the XML file is hereby incorporated by reference in its entirety.

### TECHNICAL FIELD

The present disclosure relates to the field of biotechnology, and more specifically, to single-chain and multi-chain chimeric antigen receptors.

### BACKGROUND

Chimeric antigen receptors (CARs) are a subclass of single-chain and multi-chain polypeptides. Chimeric antigen receptors typically include, e.g., an antigen-binding domain, a transmembrane domain, and one or more signaling domains. CARs have been investigated for use in the clinical treatment of different cancers, e.g., hematological cancers, in mammals.

### SUMMARY

The present disclosure is based, at least in part, on the discovery that chimeric antigen-receptors that have a specific combination of intracellular signaling domains, when expressed in a T-cell, demonstrated improved T-cell activation, improved target cell killing, and improved cytokine secretion including IL-2, interferon-gamma, and TNFα, as compared to a T-cell that expresses a chimeric antigen receptor that includes only includes the intracellular signaling domains from CD3 and CD3ξ.

Provided herein are single-chain chimeric antigen receptors that include: an extracellular antigen-binding domain; a transmembrane domain; a first intracellular signaling domain from DAP-10 or DAP12; a second intracellular signaling domain from a protein selected from the group of: 4-1BB, CD27, OX40, CD40, CD28, GITR, CD2, CD5, ICAM-1, CD11a, Lck, TNFR-I, TNFR-II, FasR, CD30, ICOS, LIGHT, NKG2C, and B7-H3; and an immunoreceptor tyrosine-based activation motif (ITAM). In some embodiments of any of the single-chain chimeric antigen receptors described herein, the extracellular antigen-binding domain is selected from the group of: a scFv, a (scFv)₂, a VHH domain, and a V_{NAR} domain. In some embodiments of any of the single-chain chimeric antigen receptors described herein, the extracellular antigen-binding domain is a scFv.

In some embodiments of any of the single-chain chimeric antigen receptors described herein, the extracellular antigen-binding domain binds specifically to a single antigen. In some embodiments of any of the single-chain chimeric antigen receptors described herein, the single antigen is a tumor antigen. In some embodiments of any of the single-chain chimeric antigen receptors described herein, the tumor antigen is selected from the group of: MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGI, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. In some embodiments of any of the single-chain chimeric antigen receptors described herein, the tumor antigen is CD19. In some embodiments of any of the single-chain chimeric antigen receptors described herein, the extracellular antigen-binding domain binds specifically to two different antigens.

In some embodiments of any of the single-chain chimeric antigen receptors described herein, the transmembrane domain is a transmembrane domain from: α chain of a T cell receptor, β chain of the T cell receptor, ξ chain of the T cell receptor, CD28, CD3ε, CD3δ, CD3γ, CD33, CD37, CD64, CD80, CD45, CD4, CD5, CD8α, CD9, CD16, CD22, CD86, or CD154. In some embodiments of any of the single-chain chimeric antigen receptors described herein, the ITAM comprises a cytoplasmic signaling sequence from CD3ξ.

In some embodiments of any of the single-chain chimeric antigen receptors described herein, when going in the N-terminal to the C-terminal direction or in the C-terminal to the N-terminal direction, the single-chain chimeric antigen receptor includes the extracellular antigen-binding domain, the transmembrane domain, the first intracellular signaling domain, the second intracellular signaling domain, and the ITAM. In some embodiments of any of the single-chain chimeric antigen receptors described herein, the transmembrane domain and the first intracellular signaling domain directly abut each other. In some embodiments of any of the single-chain chimeric antigen receptors described herein, the transmembrane domain and the first intracellular signaling domain are separated by 1 to 500 amino acids (e.g., 1 to 250 amino acids, or 1 to 50 amino acids). In some embodiments of any of the single-chain chimeric antigen receptors described herein, the first intracellular signaling domain and the second intracellular signaling domain directly abut each other. In some embodiments of any of the single-chain chimeric antigen receptors described herein, the first intracellular signaling domain and the second intracellular signaling domain are separated by 1 to 500 amino acids (e.g., 1 to 250 amino acids, or 1 to 50 amino acids). In some embodiments of any of the single-chain chimeric antigen receptors described herein, the second intracellular signaling domain and the ITAM directly abut each other. In some embodiments of any of the single-chain chimeric antigen receptors described herein, the second intracellular signaling domain and the ITAM are separated by 1 to 500 amino acids (e.g., 1 to 250 amino acids, or 1 to 50 amino acids).

In some embodiments of any of the single-chain chimeric antigen receptors described herein, when going in the N-terminal to the C-terminal direction or in the C-terminal to the N-terminal direction, the single-chain chimeric antigen receptor includes the extracellular antigen-binding domain, the transmembrane domain, the second intracellular signaling domain, the first intracellular signaling domain, and the ITAM.

In some embodiments of any of the single-chain chimeric antigen receptors described herein, when going in the N-terminal to the C-terminal direction or in the C-terminal to the N-terminal direction, the single-chain chimeric antigen receptor includes the extracellular antigen-binding domain, the transmembrane domain, the first intracellular signaling domain, the ITAM, and the second intracellular signaling domain.

In some embodiments of any of the single-chain chimeric antigen receptors described herein, when going in the N-terminal to the C-terminal direction or in the C-terminal to the N-terminal direction, the single-chain chimeric antigen receptor includes the extracellular antigen-binding domain, the transmembrane domain, the second intracellular signaling domain, the ITAM, and the first intracellular signaling domain. In some embodiments of any of the single-chain chimeric antigen receptors described herein, when going to the N-terminal to the C-terminal direction or in the C-terminal to the N-terminal direction, the single-chain chimeric antigen receptor includes the extracellular antigen-binding domain, the transmembrane domain, the ITAM, the first intracellular signaling domain, and the second intracellular signaling domain.

In some embodiments of any of the single-chain chimeric antigen receptors described herein, when going to the N-terminal to the C-terminal direction or in the C-terminal to the N-terminal direction, the single-chain chimeric antigen receptor includes the extracellular antigen-binding domain, the transmembrane domain, the ITAM, the second intracellular signaling domain, and the first intracellular signaling domain.

In some embodiments of any of the single-chain chimeric antigen receptors described herein, the extracellular antigen-binding domain and the transmembrane domain directly abut each other. In some embodiments of any of the single-chain chimeric antigen receptors described herein, the extracellular antigen-binding domain and the transmembrane domain are separated by 1 to 500 amino acids (e.g., 1 to 250 amino acids, or 1 to 50 amino acids).

In some embodiments of any of the single-chain chimeric antigen receptors described herein, the first intracellular signaling domain is from DAP-10. In some embodiments of any of the single-chain chimeric antigen receptors described herein, the first intracellular signaling domain is from DAP-12.

Also provided herein are nucleic acis that include a nucleotide sequence encoding any of the single-chain chimeric antigen receptors described herein. Also provided herein are vectors that include any of the nucleic acids described herein that include a nucleotide sequence encoding any of the single-chain chimeric antigen receptors described herein.

Also provided herein are mammalian cells that include any of the vectors described herein. In some embodiments of any of the mammalian cells described herein, the mammalian cell is a T cell. In some embodiments of any of the mammalian cells described herein, the mammalian cell is selected from the group of: a CD8⁺ T cell, a CD4⁺ T cell, a memory T cell, a Treg cell, natural killer T cell, B cell, and a macrophage/monocyte. In some embodiments of any of the mammalian cells described herein, the mammalian cell is a mammalian cell obtained from a subject. In some embodiments of any of the mammalian cells described herein, the subject is diagnosed or identified as having a cancer. In some embodiments of any of the mammalian cells described herein, the subject is human.

Also provided herein are pharmaceutical compositions that include any of the mammalian cells described herein and a pharmaceutically acceptable carrier. Also provided herein are kits that include any of the pharmaceutical compositions described herein.

Also provided herein are pharmaceutical compositions that include any of the nucleic acids described herein or any of the vectors described herein, and a pharmaceutically acceptable carrier. Also provided herein are kits that include any of the pharmaceutical compositions described herein.

Also provided herein are methods of generating a chimeric antigen receptor-expressing cell, the method comprising introducing into a mammalian cell any of the nucleic acids described herein or any of the vectors described herein. In some embodiments of any of the methods described herein, the mammalian cell is a human cell. In some embodiments of any of the methods described herein, the mammalian cell is a cell selected from the group of: a CD8+ T cell, a CD4+ T cell, a memory T cell, a Treg cell, natural killer T cell, B cell, and a macrophage/monocyte. In some embodiments of any of the methods described herein, the mammalian cell is a mammalian cell obtained from a subject. In some embodiments of any of the methods described herein, the subject is diagnosed or identified as having a cancer. Some embodiments of any of the methods described herein further include, after the introducing step: culturing the cell in a liquid culture medium. Some embodiments of any of the methods described herein further include, before the introducing step: obtaining the mammalian cell from the subject.

Also provided herein are methods of treating a cancer in a subject that include administering a therapeutically effective amount of any of the mammalian cells described herein to the subject. Some embodiments of any of the methods described herein further include, prior to the administering step, obtaining an initial cell from the subject; and introducing any of the nucleic acids described herein or any of the vectors described herein into the initial cell, to yield the mammalian cell that is administered to the subject. Some embodiments of any of the methods described herein further include, between the introducing step and the administering step, a step of culturing the cell that is administered to the subject in a liquid culture medium. In some embodiments of any of the methods described herein, the subject is human.

Also provided herein are multi-chain chimeric antigen receptors that include at least one first polypeptide including: a transmembrane domain; a first intracellular signaling domain from DAP-10 or DAP-12; a second intracellular signaling domain from a protein selected from the group of: 4-1BB, CD27, OX40, CD40, CD28, GITR, CD2, CD5, ICAM-1, CD11a, Lck, TNFR-I, TNFR-II, FasR, CD30, ICOS, LIGHT, NKG2C, and B7-H3; and an immunoreceptor tyrosine-based activation motif (ITAM). In some embodiments of any of the multi-chain chimeric antigen receptors described herein, the transmembrane domain is a transmembrane domain from: α chain of a T cell receptor, β chain of the T cell receptor, ξ chain of the T cell receptor, CD28, CD3ε, CD3δ, CD3γ, CD33, CD37, CD64, CD80, CD45, CD4, CD5, CD8α, CD9, CD16, CD22, CD86, or CD154. In some embodiments of any of the multi-chain chimeric antigen receptors described herein, the ITAM includes a cytoplasmic signaling sequence from CD3ξ.

In some embodiments of any of the multi-chain chimeric antigen receptors described herein, when going in the N-terminal to the C-terminal direction or in the C-terminal to the N-terminal direction, the at least one first polypeptide includes the transmembrane domain, the first intracellular signaling domain, the second intracellular signaling domain, and the ITAM. In some embodiments of any of the multi-chain chimeric antigen receptors described herein, the transmembrane domain and the first intracellular signaling domain directly abut each other. In some embodiments of any of the multi-chain chimeric antigen receptors described herein, the transmembrane domain and the first intracellular signaling domain are separated by 1 to 500 amino acids (e.g., 1 to 250 amino acids, or 1 to 50 amino acids). In some embodiments of any of the multi-chain chimeric antigen receptors described herein, the first intracellular signaling domain and the second intracellular signaling domain directly abut each other. In some embodiments of any of the multi-chain chimeric antigen receptors described herein, the first intracellular signaling domain and the second intracellular signaling domain are separated by 1 to 500 amino acids (e.g., 1 to 250 amino acids, or 1 to 50 amino acids). In some embodiments of any of the multi-chain chimeric antigen receptors described herein, the second intracellular signaling domain and the ITAM directly abut each other. In some embodiments of any of the multi-chain chimeric antigen receptors described herein, the second intracellular signaling domain and the ITAM are separated by 1 to 500 amino acids (e.g., 1 to 250 amino acids, or 1 to 50 amino acids).

In some embodiments of any of the multi-chain chimeric antigen receptors described herein, when going in the N-terminal to the C-terminal direction or in the C-terminal to the N-terminal direction, the at least one first polypeptide includes the transmembrane domain, the second intracellular signaling domain, the first intracellular signaling domain, and the ITAM.

In some embodiments of any of the multi-chain chimeric antigen receptors described herein, when going in the N-terminal to the C-terminal direction or in the C-terminal to the N-terminal direction, the at least one first polypeptide includes the transmembrane domain, the first intracellular signaling domain, the ITAM, and the second intracellular signaling domain.

In some embodiments of any of the multi-chain chimeric antigen receptors described herein, when going in the N-terminal to the C-terminal direction or in the C-terminal to the N-terminal direction, the at least one first polypeptide includes the transmembrane domain, the second intracellular signaling domain, the ITAM, and the first intracellular signaling domain.

In some embodiments of any of the multi-chain chimeric antigen receptors described herein, when going to the N-terminal to the C-terminal direction or in the C-terminal to the N-terminal direction, the at least one first polypeptide includes the transmembrane domain, the ITAM, the first intracellular signaling domain, and the second intracellular signaling domain.

In some embodiments of any the multi-chain chimeric antigen receptors described herein, when going to the N-terminal to the C-terminal direction or in the C-terminal to the N-terminal direction, the at least one first polypeptide includes the transmembrane domain, the ITAM, the second intracellular signaling domain, and the first intracellular signaling domain.

In some embodiments of any of the multi-chain chimeric antigen receptors described herein, the first intracellular signaling domain is from DAP-10. In some embodiments of any of the multi-chain chimeric antigen receptors described herein, the first intracellular signaling domain is from DAP-12.

Also provided herein are nucleic acids that encode any of the multi-chain chimeric antigen receptors described herein. Also provided herein are sets of nucleic acids that together encode any of the multi-chain chimeric antigen receptors described herein.

Also provided herein are mammalian cells that include any of the nucleic acids described herein that encode any of the multi-chain chimeric antigen receptors described herein. Also provided herein are mammalian cells that include any of the sets of nucleic acids described herein that together encode any of the multi-chain chimeric antigen receptors described herein. In some embodiments of any of the mammalian cells described herein, the mammalian cell is a T cell. In some embodiments of any of the mammalian cells described herein, the mammalian cell is selected from the group of: a CD8⁺ T cell, a CD4⁺ T cell, a memory T cell, a Treg cell, natural killer T cell, B cell, and a macrophage/monocyte. In some embodiments of any of the mammalian cells described herein, the mammalian cell is a mammalian cell obtained from a subject. In some embodiments of any of the mammalian cells described herein, the subject is diagnosed or identified as having a cancer. In some embodiments of of any of the mammalian cells described herein, the subject is human.

Also provided herein are pharmaceutical compositions that include any of the mammalian cells described herein and a pharmaceutically acceptable carrier. Also provided herein are kits that include any of the pharmaceutical compositions described herein.

Also provided herein are pharmaceutical compositions that include any of the nucleic acids described herein that encode any of the multi-chain chimeric antigen receptors described herein, or any of the sets of nucleic acids described herein that together encode any of the multi-chain chimeric antigen receptors described herein, and a pharmaceutically acceptable carrier. Also provided herein are kits that include any of the pharmaceutical compositions described herein.

Also provided herein are methods of generating a multi-chain chimeric antigen receptor-expressing cell that include introducing into a mammalian cell any of the nucleic acids described herein that encode any of the multi-chain chimeric antigen receptors described herein, or any of the sets of nucleic acids described herein that encode any of the multi-chain chimeric antigen receptors described herein. In some embodiments of any of the methods described herein, the mammalian cell is a human cell. In some embodiments of any of the methods described herein, the mammalian cell is a cell selected from the group consisting of: a CD8+ T cell, a CD4+ T cell, a memory T cell, a Treg cell, natural killer T cell, B cell, and a macrophage/monocyte. In some embodiments of any of the methods described herein, the mammalian cell is a mammalian cell obtained from a subject. In some embodiments of any of the methods described herein, the subject is diagnosed or identified as having a cancer. Some embodiments of any of the methods described herein further include, after the introducing step, culturing the cell in a liquid culture medium. Some embodiments of any of the methods described herein further include, before the introducing step, obtaining the mammalian cell from the subject.

Also provided herein are methods of treating cancer in a subject that include administering a therapeutically effective amount of any of the mammalian cell described herein to the subject. Some embodiments of any of the methods described herein further include, prior to the administering step, obtaining an initial cell from the subject; and
introducing any of the nucleic acids descried herein that encode any of the multi-chain chimeric antigen receptors described herein or any of the sets of nucleic acids described herein that together encode any of the multi-chain chimeric antigen receptors described herein into the initial cell, to yield the mammalian cell that is administered to the subject. Some embodiments of any of the methods described herein further include, between the introducing step and the administering step, a step of culturing the cell that is administered to the subject in a liquid culture medium. In some embodiments of any of the methods described herein, the subject is human.

Also provided herein are single-chain chimeric antigen receptors that include: an extracellular antigen-binding domain; a transmembrane domain; an intracellular signaling domain from a protein selected from the group of: 4-1BB, CD27, OX40, CD40, CD28, GITR, DAP-10, DAP-12, CD2, CD5, ICAM-1, CD11a, Lck, TNFR-I, TNFR-II, FasR, CD30, ICOS, LIGHT, NKG2C, and B7-H3; and an immunoreceptor tyrosine-based activation motif (ITAM). In some embodiments of any of the single-chain chimeric antigen receptors described herein, the extracellular antigen-binding domain is selected from the group consisting of: a scFv, a (scFv)₂, a VHH domain, and a V_{NAR} domain. In some embodiments of any of the single-chain chimeric antigen receptors described herein, the extracellular antigen-binding domain is a scFv. In some embodiments of any of the single-chain chimeric antigen receptors described herein, the extracellular antigen-binding domain binds specifically to a single antigen. In some embodiments of any of the single-chain chimeric antigen receptors described herein, the single antigen is a tumor antigen. In some embodiments of any of the single-chain chimeric antigen receptors described herein, the tumor antigen is selected from the group of: MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGI, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. In some embodiments of any of the single-chain chimeric antigen receptors described herein, the tumor antigen is CD19. In some embodiments of any of the single-chain chimeric antigen receptors described herein, the extracellular antigen-binding domain binds specifically to two different antigens.

In some embodiments of any of the single-chain chimeric antigen receptors described herein, the transmembrane domain is a transmembrane domain from: α chain of a T cell receptor, β chain of the T cell receptor, ξ chain of the T cell receptor, CD28, CD3ε, CD3δ, CD3γ, CD33, CD37, CD64, CD80, CD45, CD4, CD5, CD8α, CD9, CD16, CD22, CD86, or CD154. In some embodiments of any of the single-chain chimeric antigen receptors described herein, the ITAM includes a cytoplasmic signaling sequence from CD3ξ.

In some embodiments of any of the single-chain chimeric antigen receptors described herein, when going in the N-terminal to the C-terminal direction or in the C-terminal to the N-terminal direction, the single-chain chimeric antigen receptor include the extracellular antigen-binding domain, the transmembrane domain, the intracellular signaling domain, and the ITAM. In some embodiments of any of the single-chain chimeric antigen receptors described herein, the transmembrane domain and the intracellular signaling domain directly abut each other. In some embodiments of any of the single-chain chimeric antigen receptors described herein, the transmembrane domain and the intracellular signaling domain are separated by 1 to 500 amino acids (e.g., 1 to 250 amino acids, or 1 to 50 amino acids). In some embodiments of any of the single-chain chimeric antigen receptors described herein, the intracellular signaling domain and the ITAM directly abut each other. In some embodiments of any of the single-cain chimeric antigen receptors described herein, the intracellular signaling domain and the ITAM are separated by 1 to 500 amino acids (e.g., 1 to 250 amino acids, or 1 to 50 amino acids).

In some embodiments of any of the single-chain chimeric antigen receptors described herein, when going in the N-terminal to the C-terminal direction or in the C-terminal to the N-terminal direction, the single-chain chimeric antigen receptor includes the extracellular antigen-binding domain, the transmembrane domain, the ITAM, and the intracellular signaling domain. In some embodiments of any of the single-chain chimeric antigen receptors described herein, the extracellular antigen-binding domain and the transmembrane domain directly abut each other. In some embodiments of any of the single-chain chimeric antigen receptors described herein, the extracellular antigen-binding domain and the transmembrane domain are separated by 1 to 500 amino acids (e.g., 1 to 250 amino acids, or 1 to 50 amino acids).

Also provided herein are nucleic acids the include a nucleotide sequence encoding any of the single-chain chimeric antigen receptors described herein. Also provided herein are vectors that include any of the nucleic acids described herein that encode any of the single-chain chimeric antigen receptors described herein.

Also provided herein are mammalian cells that include any of the vectors described herein. In some embodiments of any of the mammalian cells described herein, the mammalian cell is a T cell. In some embodiments of any of the mammalian cells described herein, the mammalian cell is selected from the group of: a CD8⁺ T cell, a CD4⁺ T cell, a memory T cell, a Treg cell, natural killer T cell, B cell, and a macrophage/monocyte. In some embodiments of any of the mammalian cells described herein, the mammalian cell is a mammalian cell obtained from a subject. In some embodiments of any of the mammalian cells described herein, the subject is diagnosed or identified as having a cancer. In some embodiments of any of the mammalian cells described herein, the subject is human.

Also provided herein are pharmaceutical compositions that include any of the mammalian cells described herein and a pharmaceutically acceptable carrier. Also provided herein are kits that include any of the pharmaceutical compositions described herein.

Also provided herein are pharmaceutical compositions that include any of the nucleic acids described herein that encode any of the single-chain chimeric antigen receptors described herein, and a pharmaceutically acceptable carrier. Also provided herein are kits that include any of the pharmaceutical compositions described herein.

Also provided herein are methods of generating a chimeric antigen receptor-expressing cell that include introducing into a mammalian cell any of the nucleic acids described herein that encode any of the single-chain chimeric antigen receptors described herein or any of the vectors described herein. In some embodiments of any of the methods described herein, the mammalian cell is a human cell. In some embodiments of any of the methods described herein, the mammalian cell is a cell selected from the group of: a CD8+ T cell, a CD4+ T cell, a memory T cell, a Treg cell, natural killer T cell, B cell, and a macrophage/monocyte. In some embodiments of any of the methods described herein, the mammalian cell is a mammalian cell obtained from a subject. In some embodiments of any of the methods described herein, the subject is diagnosed or identified as having a cancer. Some embodiments of any of the methods described herein further include, after the introducing step, culturing the cell in a liquid culture medium. Some embodiments of any of the methods described herein further include, before the introducing step, obtaining the mammalian cell from the subject.

Also provided herein are methods of treating a cancer in a subject that include administering a therapeutically effective amount of any of the mammalian cells described herein. Some embodiments of any of the methods described herein further include, prior to the administering step, obtaining an initial cell from the subject; and introducing any of the nucleic acids described herein that encode any of the single-chain chimeric antigen receptors described herein or any of the vectors described herein into the initial cell, to yield the mammalian cell that is administered to the subject. Some embodiments of any of the methods described herein further include, between the introducing step and the administering step, a step of culturing the cell that is administered to the subject in a liquid culture medium. In some embodiments of any of the methods described herein, the subject is human.

Also provided herein are multi-chain chimeric antigen receptors that include at least one first polypeptide including: a transmembrane domain; an intracellular signaling domain from a protein selected from the group of: 4-1BB, CD27, OX40, CD40, CD28, GITR, DAP-10, DAP-12, CD2, CD5, ICAM-1, CD11a, Lck, TNFR-I, TNFR-II, FasR, CD30, ICOS, LIGHT, NKG2C, and B7-H3; and an immunoreceptor tyrosine-based activation motif (ITAM). In some embodiments of any of the multi-chain chimeric antigen receptors described herein, the transmembrane domain is a transmembrane domain from: α chain of a T cell receptor, β chain of the T cell receptor, ξ chain of the T cell receptor, CD28, CD3ε, CD3δ, CD3γ, CD33, CD37, CD64, CD80, CD45, CD4, CD5, CD8α, CD9, CD16, CD22, CD86, or CD154. In some embodiments of any of the multi-chain chimeric antigen receptors described herein, the ITAM includes a cytoplasmic signaling sequence from CD3ξ.

In some embodiments of any of the multi-chain chimeric antigen receptors described herein, when going in the N-terminal to the C-terminal direction or in the C-terminal to the N-terminal direction, the at least one first polypeptide includes the transmembrane domain, the intracellular signaling domain, and the ITAM. In some embodiments of any of the multi-chain chimeric antigen receptors described herein, the transmembrane domain and the intracellular signaling domain directly abut each other. In some embodiments of any of the multi-chain chimeric antigen receptors described herein, the transmembrane domain and the intracellular signaling domain are separated by 1 to 500 amino acids (e.g., 1 to 250 amino acids, or 1 to 50 amino acids). In some embodiments of any of the multi-chain chimeric antigen receptors described herein, the intracellular signaling domain and the ITAM directly abut each other. In some embodiments of any of the multi-chain chimeric antigen receptors described herein, the intracellular signaling domain and the ITAM are separated by 1 to 500 amino acids (e.g., 1 to 250 amino acids, or 1 to 50 amino acids).

In some embodiments of any of the multi-chain chimeric antigen receptors described herein, when going in the N-terminal to the C-terminal direction or in the C-terminal to the N-terminal direction, the at least one first polypeptide includes the transmembrane domain, the ITAM, and the intracellular signaling domain.

Also provided herein are nucleic acids that encode any of the multi-chain chimeric antigen receptors described herein. Also provided herein are sets of nucleic acids that together encode any of the multi-chain chimeric antigen receptors described herein.

Also provided herein are mammalian cells that include any of the nucleic acids described herein that encode any of the multi-chain chimeric antigen receptors described herein. Also provided herein are mammalian cells that include any of the sets of nucleic acids described herein that together encode any of the multi-chain chimeric antigen receptors described herein. In some embodiments of any of the mammalian cells described herein, the mammalian cell is a T cell. In some embodiments of any of the mammalian cells described herein, the mammalian cell is selected from the group of: a CD8⁺ T cell, a CD4⁺ T cell, a memory T cell, a Treg cell, natural killer T cell, B cell, and a macrophage/monocyte. In some embodiments of any of the mammalian cells described herein, the mammalian cell is a mammalian cell obtained from a subject. In some embodiments of any of the mammalian cells described herein, the subject is diagnosed or identified as having a cancer. In some embodiments of any of the mammalian cells described herein, the subject is human.

Also provided herein are pharmaceutical compositions that include any of the mammalian cells described herein and a pharmaceutically acceptable carrier. Also provided herein are kits that include any of the pharmaceutical compositions described herein.

Also provided herein are pharmaceutical compositions that include any of the nucleic acids described herein that encode any of the multi-chain chimeric antigen receptors described herein or any of the sets of nucleic acids described herein that together encode any of the multi-chain chimeric antigen receptors described herein, and a pharmaceutically acceptable carrier. Also provided are kits that include any of the pharmaceutical compositions described herein.

Also provided herein are methods of generating a chimeric antigen receptor-expressing cell that include introducing into a mammalian cell any of the nucleic acids described herein that encode any of the multi-chain chimeric antigen receptors described herein or any of the sets of nucleic acids described herein that encode any of the multi-chain chimeric antigen receptors described herein. In some embodiments of any of the methods described herein, the mammalian cell is a human cell. In some embodiments of any of the methods described herein, the mammalian cell is a cell selected from the group consisting of: a CD8+ T cell, a CD4+ T cell, a memory T cell, a Treg cell, natural killer T cell, B cell, and a macrophage/monocyte. In some embodiments of any of the methods described herein, the mammalian cell is a mammalian cell obtained from a subject. In some embodiments of any of the methods described herein, the subject is diagnosed or identified as having a cancer. Some embodiments of any of the methods described herein further include, after the introducing step, culturing the cell in a liquid culture medium. Some embodiments of any of the methods described herein further include, before the introducing step, obtaining the mammalian cell from the subject.

Also provided herein are methods of treating a cancer in a subject that include administering a therapeutically effective amount of any of the mammalian cells described herein. Some embodiments of any of the methods described herein further include, prior to the administering step, obtaining an initial cell from the subject; and introducing any of the nucleic acids described herein that encode any of the multi-chain chimeric antigen receptors described herein, of any of the sets of nucleic acids described herein that encode any of the multi-chain chimeric antigen receptors described herein into the initial cell, to yield the mammalian cell that is administered to the subject. Some embodiments of any of the methods described herein further include, between the introducing step and the administering step, a step of culturing the cell that is administered to the subject in a liquid culture medium. In some embodiments of any of the methods described herein, the subject is human.

The use of the term “a” before a noun is meant “one or more” of the particular noun. For example, the phrase “a mammalian cell” means “one or more mammalian cell.”

The terms “chimeric antigen receptor” and “CAR” are used interchangeably herein, and refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. CAR molecules and derivatives thereof (e.g., CAR variants) are described, e.g., in PCT Application No. US2014/016527; Fedorov et al. Sci Transl Med (2013); 5 (215): 215ra172; Glienke et al. Front Pharmacol (2015) 6:21; Kakarla & Gottschalk 52 Cancer J (2014)₂₀ (2): 151-5; Riddell et al. Cancer J (2014)₂₀ (2): 141-4; Pegram et al. Cancer J (2014)₂₀ (2): 127-33; Cheadle et al. Immunol Rev (2014)₂₅₇ (1): 91-106; Barrett et al. Annu Rev Med (2014) 65:333-47; Sadelain et al. Cancer Discov (2013) 3 (4): 388-98; Cartellieri et al., J Biomed Biotechnol (2010) 956304; the disclosures of which are incorporated herein by reference in their entirety. A CAR can be a single-chain chimeric antigen receptor or a multi-chain chimeric antigen receptor.

The term “single-chain chimeric antigen receptor” means a chimeric antigen receptor that is a single-chain polypeptide.

The term “multi-chain chimeric antigen receptor” means a chimeric antigen receptor including two or more single-chain polypeptides.

The term “transmembrane domain” means a domain of a polypeptide that includes at least one contiguous amino acid sequence that traverses a lipid bilayer when present in the corresponding endogenous polypeptide when expressed in a mammalian cell. For example, a transmembrane domain can include one, two, three, four, five, six, seven, eight, nine, or ten contiguous amino acid sequences that each traverse a lipid bilayer when present in the corresponding endogenous polypeptide when expressed in a mammalian cell. As is known in the art, a transmembrane domain can, e.g., include at least one (e.g., two, three, four, five, six, seven, eight, nine, or ten) contiguous amino acid sequence (that traverses a lipid bilayer when present in the corresponding endogenous polypeptide when expressed in a mammalian cell) that has α-helical secondary structure in the lipid bilayer. In some embodiments, a transmembrane domain can include two or more contiguous amino acid sequences (that each traverse a lipid bilayer when present in the corresponding endogenous polypeptide when expressed in a mammalian cell) that form a β-barrel secondary structure in the lipid bilayer. Non-limiting examples of transmembrane domains are described herein. Additional examples of transmembrane domains are known in the art.

The term “antigen-binding domain” means a domain that binds specifically to a target antigen. In some examples, an antigen-binding domain can be formed from the amino acids present within a single-chain polypeptide. In other examples, an antigen-binding domain can be formed from amino acids present within a first single-chain polypeptide and the amino acids present in one or more additional single-chain polypeptides (e.g., a second single-chain polypeptide). Non-limiting examples of antigen-binding domains are described herein, including, without limitation, scFvs, or LBDs of growth factors. Additional examples of antigen-binding domains are known in the art.

As used herein, the term “antigen” refers generally to a binding partner specifically recognized by an antigen-binding domain described herein. Exemplary antigens include different classes of molecules, such as, but not limited to, polypeptides and peptide fragments thereof, small molecules, lipids, carbohydrates, and nucleic acids. Non-limiting examples of antigen or antigens that can be specifically bound by any of the antigen-binding domains are described herein. Additional examples of antigen or antigens that can be specifically bound by any of the antigen-binding domains are known in the art.

The term “intracellular signaling domain” means an intracellular signaling domain from an endogenous signaling transmembrane polypeptide expressed in an immune cell (e.g., a T lymphocyte) that promotes downstream immune cell signaling (e.g., T-cell receptor signaling) and/or immune cell activation (e.g., T cell activation). Non-limiting examples of intracellular signaling domains are described herein. Additional examples of intracellular signaling domains are known in the art. See, e.g., Chen et al., Nature Reviews Immunol. 13:227-242, 2013.

The term “immunoreceptor tyrosine-based activation motif” or “ITAM)” means an amino acid motif that includes a four amino-acid consensus sequence of a tyrosine separated from a leucine or an isoleucine by two other amino acids (YxxL/I). The tyrosine residue in the four-amino acid consensus sequence becomes phosphorylated following interaction of a signaling pathway kinase (e.g., a lymphocyte signaling pathway kinase). Non-limiting examples of ITAMs are described herein. Additional examples of ITAMs are known in the art.

The phrase “treatment of cancer” means a reduction in the number, frequency, or severity of one or more (e.g., two, three, four, or five) symptoms of a cancer in a subject having a cancer. Non-limiting symptoms of cancer are described herein. Additional symptoms of cancer are known in the art.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 shows the percent lysis of cell lines after co-culturing with CAR-T cells expressing various CAR constructs. The X axis shows the intracellular signaling domain(s) and ITAM in each tested construct. The Y axis shows the percentage of target cells that were lysed in the presence of the various CAR-T cells expressing the indicated CARs. The sequences of the various intracellular sequences of the CAR constructs are described in Example 1. UT=Untransduced.

FIG. 2 shows CD69 expression of CAR-T cells expressing various tested CAR constructs. The X axis shows the intracellular signaling domain(s) and ITAM in each tested construct. The Y axis shows the expression of CD69. MFI=median fluorescence intensity. The sequences of the various intracellular sequences of the CAR constructs are described in Example 1. UT=Untransduced.

FIGS. 3A-C show cytokine production of CAR-T cells expressing various CAR constructs. FIG. 3A shows TNF-alpha production. FIG. 3B shows interferon gamma production. FIG. 3C shows interleukin 2 production. The X axes of FIGS. 3A-C show the intracellular signaling domain(s) and ITAM in each tested construct. The Y axes of FIGS. 3A-C show the expression in pg/ml of TNF-alpha, interferon gamma, and interleukin 2 cytokines, respectively. The sequences of the various intracellular sequences of the CAR constructs are described in Example 1. UT=Untransduced.

### DETAILED DESCRIPTION

Provided herein are single-chain chimeric antigen receptors that include: an extracellular antigen-binding domain; a transmembrane domain; a first intracellular signaling domain from DAP-10 or DAP-12, a second intracellular signaling domain from a protein selected from the group of 4-1BB, CD27, OX40, CD40, CD28, GITR, CD2, CD5, ICAM-1, CD11a, Lck, TNFR-I, TNFR-II, FasR, CD30, ICOS, LIGHT, NKG2C, and B7-H3; and an immunoreceptor tyrosine-based activation motif (ITAM).

Also provided herein are multi-chain chimeric antigen receptors that comprise at least one first polypeptide including: a transmembrane domain; a first intracellular signaling domain from DAP-10 or DAP-12; a second intracellular signaling domain from a protein selected from the group of 4-1BB, CD27, OX40, CD40, CD28, GITR, CD2, CD5, ICAM-1, CD11a, Lck, TNFR-I, TNFR-II, FasR, CD30, ICOS, LIGHT, NKG2C, and B7-H3; and an ITAM.

Also provided herein are single-chain chimeric antigen receptors that include: an extracellular antigen-binding domain; a transmembrane domain; an intracellular signaling domain from a protein selected from the group of 4-1BB, CD27, OX40, CD40, CD28, GITR, DAP-10, DAP-12, CD2, CD5, ICAM-1, CD11a, Lck, TNFR-I, TNFR-II, FasR, CD30, ICOS, LIGHT, NKG2C, and B7-H3; and an ITAM. Some embodiments of any of the single-chain chimeric antigen receptors described herein can include, in addition to the extracellular antigen-binding domain, the transmembrane domain, and the ITAM, one of the following combinations of intracellular signaling domains: (i) DAP12-OX40, (ii) DAP10-OX40, (iii) DAP12-CD40, (iv) DAP10-CD28, (v) DAP12-CD28, (vi) DAP10-41BB, (vii) DAP10-CD27, (viii) DAP10-CD40, (ix) DAP10-GITR, (x) DAP12-41BB, (xi) DAP12-CD27, (xii) DAP12-GITR, and (xiii) CD2-DAP12.

Also provided herein are multi-chain chimeric antigen receptors that comprise at least one first polypeptide including: an extracellular antigen-binding domain; a transmembrane domain; an intracellular signaling domain from a protein selected from the group of 4-1BB, CD27, OX40, CD40, CD28, GITR, DAP-10, DAP-12, CD2, CD5, ICAM-1, CD11a, Lck, TNFR-I, TNFR-II, FasR, CD30, ICOS, LIGHT, NKG2C, and B7-H3; and an ITAM. Some embodiments of any of the multi-chain chimeric antigen receptors described herein comprise at least one first polypeptide that include, in addition to the extracellular antigen-binding domain, the transmembrane domain, and the ITAM, one of the following combinations of intracellular signaling domains: (i) DAP12-OX40, (ii) DAP10-OX40, (iii) DAP12-CD40, (iv) DAP10-CD28, (v) DAP12-CD28, (vi) DAP10⁻⁴¹BB, (vii) DAP10-CD27, (viii) DAP10-CD40, (ix) DAP10-GITR, (x) DAP12-41BB, (xi) DAP12-CD27, (xii) DAP12-GITR, and (xiii) CD2-DAP12.

Also provided herein are nucleic acids encoding any of the single-chain chimeric antigen receptors or any of the multi-chain chimeric antigen receptors described herein; vectors including any of these nucleic acids; mammalian cells including any of these vectors; and methods of treating a cancer in a subject by administering a mammalian cell (e.g., a T-cell) expressing any of the single-chain chimeric antigen receptors or any of the multi-chain chimeric antigen receptors described herein.

Non-limiting aspects of these single-chain chimeric antigen receptors, multi-chain chimeric antigen receptors, nucleic acids, vectors, mammalian cells, and methods are described below, and can be used in any combination without limitation. Additional aspects of these single-chain chimeric antigen receptors, multi-chain chimeric antigen receptors, nucleic acids, vectors, mammalian cells, and methods are known in the art.

In some embodiments, the compositions and/or methods disclosed herein can be used to selectively kill cancer cells in a subject. In some embodiments, a population of T-cells expressing any of the single-chain chimeric antigen receptors or any of the multi-chain chimeric antigen receptors described herein can have increased (e.g., a 1% to 300%, a 1% to 280%, a 1% to 260%, a 1% to 240%, a 1% to 220%, a 1% to 200%, a 1% to 190%, a 1% to 180%, a 1% to 170%, a 1% to 160%, a 1% to 150%, a 1% to 140%, a 1% to 130%, a 1% to 120%, a 1% to 110%, a 1% to 100%, a 1% to 95%, a 1% to 90%, a 1% to 85%, a 1% to 80%, a 1% to 75%, a 1% to 70%, a 1% to 65%, a 1% to 60%, a 1% to 55%, a 1% to 50%, a 1% to 45%, a 1% to 40%, a 1% to 35%, a 1% to 30%, a 1% to 25%, a 1% to 20%, a 1% to 15%, a 1% to 10%, a 1% to 5%, a 2% to 300%, a 2% to 280%, a 2% to 260%, a 2% to 240%, a 2% to 220%, a 2% to 200%, a 2% to 190%, a 2% to 180%, a 2% to 170%, a 2% to 160%, a 2% to 150%, a 2% to 140%, a 2% to 130%, a 2% to 120%, a 2% to 110%, a 2% to 100%, a 2% to 95%, a 2% to 90%, a 2% to 85%, a 2% to 80%, a 2% to 75%, a 2% to 70%, a 2% to 65%, a 2% to 60%, a 2% to 55%, a 2% to 50%, a 2% to 45%, a 2% to 40%, a 2% to 35%, a 2% to 30%, a 2% to 25%, a 2% to 20%, a 2% to 15%, a 2% to 10%, a 2% to 5%, a 3% to 300%, a 3% to 280%, a 3% to 260%, a 3% to 240%, a 3% to 220%, a 3% to 200%, a 3% to 190%, a 3% to 180%, a 3% to 170%, a 3% to 160%, a 3% to 150%, a 3% to 140%, a 3% to 130%, a 3% to 120%, a 3% to 110%, a 3% to 100%, a 3% to 95%, a 3% to 90%, a 3% to 85%, a 3% to 80%, a 3% to 75%, a 3% to 70%, a 3% to 65%, a 3% to 60%, a 3% to 55%, a 3% to 50%, a 3% to 45%, a 3% to 40%, a 3% to 35%, a 3% to 30%, a 3% to 25%, a 3% to 20%, a 3% to 15%, a 3% to 10%, a 3% to 5%, a 5% to 300%, a 5% to 280%, a 5% to 260%, a 5% to 240%, a 5% to 220%, a 5% to 200%, a 5% to 190%, a 5% to 180%, a 5% to 170%, a 5% to 160%, a 5% to 150%, a 5% to 140%, a 5% to 130%, a 5% to 120%, a 5% to 110%, a 5% to 100%, a 5% to 95%, a 5% to 90%, a 5% to 85%, a 5% to 80%, a 5% to 75%, a 5% to 70%, a 5% to 65%, a 5% to 60%, a 5% to 55%, a 5% to 50%, a 5% to 45%, a 5% to 40%, a 5% to 35%, a 5% to 30%, a 5% to 25%, a 5% to 20%, a 5% to 15%, a 5% to 10%, a 10% to 300%, a 10% to 280%, a 10% to 260%, a 10% to 240%, a 10% to 220%, a 10% to 200%, a 10% to 190%, a 10% to 180%, a 10% to 170%, a 10% to 160%, a 10% to 150%, a 10% to 140%, a 10% to 130%, a 10% to 120%, a 10% to 110%, a 10% to 100%, a 10% to 95%, a 10% to 90%, a 10% to 85%, a 10% to 80%, a 10% to 75%, a 10% to 70%, a 10% to 65%, a 10% to 60%, a 10% to 55%, a 10% to 50%, a 10% to 45%, a 10% to 40%, a 10% to 35%, a 10% to 30%, a 10% to 25%, a 10% to 20%, a 10% to 15%, a 15% to 300%, a 15% to 280%, a 15% to 260%, a 15% to 240%, a 15% to 220%, a 15% to 200%, a 15% to 190%, a 15% to 180%, a 15% to 170%, a 15% to 160%, a 15% to 150%, a 15% to 140%, a 15% to 130%, a 15% to 120%, a 15% to 110%, a 15% to 100%, a 15% to 95%, a 15% to 90%, a 15% to 85%, a 15% to 80%, a 15% to 75%, a 15% to 70%, a 15% to 65%, a 15% to 60%, a 15% to 55%, a 15% to 50%, a 15% to 45%, a 15% to 40%, a 15% to 35%, a 15% to 30%, a 15% to 25%, a 15% to 20%, a 20% to 300%, a 20% to 280%, a 20% to 260%, a 20% to 240%, a 20% to 220%, a 20% to 200%, a 20% to 190%, a 20% to 180%, a 20% to 170%, a 20% to 160%, a 20% to 150%, a 20% to 140%, a 20% to 130%, a 20% to 120%, a 20% to 110%, a 20% to 100%, a 20% to 95%, a 20% to 90%, a 20% to 85%, a 20% to 80%, a 20% to 75%, a 20% to 70%, a 20% to 65%, a 20% to 60%, a 20% to 55%, a 20% to 50%, a 20% to 45%, a 20% to 40%, a 20% to 35%, a 20% to 30%, a 20% to 25%, a 25% to 300%, a 25% to 280%, a 25% to 260%, a 25% to 240%, a 25% to 220%, a 25% to 200%, a 25% to 190%, a 25% to 180%, a 25% to 170%, a 25% to 160%, a 25% to 150%, a 25% to 140%, a 25% to 130%, a 25% to 120%, a 25% to 110%, a 25% to 100%, a 25% to 95%, a 25% to 90%, a 25% to 85%, a 25% to 80%, a 25% to 75%, a 25% to 70%, a 25% to 65%, a 25% to 60%, a 25% to 55%, a 25% to 50%, a 25% to 45%, a 25% to 40%, a 25% to 35%, a 25% to 30%, a 30% to 300%, a 30% to 280%, a 30% to 260%, a 30% to 240%, a 30% to 220%, a 30% to 200%, a 30% to 190%, a 30% to 180%, a 30% to 170%, a 30% to 160%, a 30% to 150%, a 30% to 140%, a 30% to 130%, a 30% to 120%, a 30% to 110%, a 30% to 100%, a 30% to 95%, a 30% to 90%, a 30% to 85%, a 30% to 80%, a 30% to 75%, a 30% to 70%, a 30% to 65%, a 30% to 60%, a 30% to 55%, a 30% to 50%, a 30% to 45%, a 30% to 40%, a 30% to 35%, a 35% to 300%, a 35% to 280%, a 35% to 260%, a 35% to 240%, a 35% to 220%, a 35% to 200%, a 35% to 190%, a 35% to 180%, a 35% to 170%, a 35% to 160%, a 35% to 150%, a 35% to 140%, a 35% to 130%, a 35% to 120%, a 35% to 110%, a 35% to 100%, a 35% to 95%, a 35% to 90%, a 35% to 85%, a 35% to 80%, a 35% to 75%, a 35% to 70%, a 35% to 65%, a 35% to 60%, a 35% to 55%, a 35% to 50%, a 35% to 45%, a 35% to 40%, a 40% to 300%, a 40% to 280%, a 40% to 260%, a 40% to 240%, a 40% to 220%, a 40% to 200%, a 40% to 190%, a 40% to 180%, a 40% to 170%, a 40% to 160%, a 40% to 150%, a 40% to 140%, a 40% to 130%, a 40% to 120%, a 40% to 110%, a 40% to 100%, a 40% to 95%, a 40% to 90%, a 40% to 85%, a 40% to 80%, a 40% to 75%, a 40% to 70%, a 40% to 65%, a 40% to 60%, a 40% to 55%, a 40% to 50%, a 40% to 45%, a 45% to 300%, a 45% to 280%, a 45% to 260%, a 45% to 240%, a 45% to 220%, a 45% to 200%, a 45% to 190%, a 45% to 180%, a 45% to 170%, a 45% to 160%, a 45% to 150%, a 45% to 140%, a 45% to 130%, a 45% to 120%, a 45% to 110%, a 45% to 100%, a 45% to 95%, a 45% to 90%, a 45% to 85%, a 45% to 80%, a 45% to 75%, a 45% to 70%, a 45% to 65%, a 45% to 60%, a 45% to 55%, a 45% to 50%, a 50% to 300%, a 50% to 280%, a 50% to 260%, a 50% to 240%, a 50% to 220%, a 50% to 200%, a 50% to 190%, a 50% to 180%, a 50% to 170%, a 50% to 160%, a 50% to 150%, a 50% to 140%, a 50% to 130%, a 50% to 120%, a 50% to 110%, a 50% to 100%, a 50% to 95%, a 50% to 90%, a 50% to 85%, a 50% to 80%, a 50% to 75%, a 50% to 70%, a 50% to 65%, a 50% to 60%, a 50% to 55%, a 55% to 300%, a 55% to 280%, a 55% to 260%, a 55% to 240%, a 55% to 220%, a 55% to 200%, a 55% to 190%, a 55% to 180%, a 55% to 170%, a 55% to 160%, a 55% to 150%, a 55% to 140%, a 55% to 130%, a 55% to 120%, a 55% to 110%, a 55% to 100%, a 55% to 95%, a 55% to 90%, a 55% to 85%, a 55% to 80%, a 55% to 75%, a 55% to 70%, a 55% to 65%, a 55% to 60%, a 60% to 300%, a 60% to 280%, a 60% to 260%, a 60% to 240%, a 60% to 220%, a 60% to 200%, a 60% to 190%, a 60% to 180%, a 60% to 170%, a 60% to 160%, a 60% to 150%, a 60% to 140%, a 60% to 130%, a 60% to 120%, a 60% to 110%, a 60% to 100%, a 60% to 95%, a 60% to 90%, a 60% to 85%, a 60% to 80%, a 60% to 75%, a 60% to 70%, a 60% to 65%, a 65% to 300%, a 65% to 280%, a 65% to 260%, a 65% to 240%, a 65% to 220%, a 65% to 200%, a 65% to 190%, a 65% to 180%, a 65% to 170%, a 65% to 160%, a 65% to 150%, a 65% to 140%, a 65% to 130%, a 65% to 120%, a 65% to 110%, a 65% to 100%, a 65% to 95%, a 65% to 90%, a 65% to 85%, a 65% to 80%, a 65% to 75%, a 65% to 70%, a 70% to 300%, a 70% to 280%, a 70% to 260%, a 70% to 240%, a 70% to 220%, a 70% to 200%, a 70% to 190%, a 70% to 180%, a 70% to 170%, a 70% to 160%, a 70% to 150%, a 70% to 140%, a 70% to 130%, a 70% to 120%, a 70% to 110%, a 70% to 100%, a 70% to 95%, a 70% to 90%, a 70% to 85%, a 70% to 80%, a 70% to 75%, a 75% to 300%, a 75% to 280%, a 75% to 260%, a 75% to 240%, a 75% to 220%, a 75% to 200%, a 75% to 190%, a 75% to 180%, a 75% to 170%, a 75% to 160%, a 75% to 150%, a 75% to 140%, a 75% to 130%, a 75% to 120%, a 75% to 110%, a 75% to 100%, a 75% to 95%, a 75% to 90%, a 75% to 85%, a 75% to 80%, a 80% to 300%, a 80% to 280%, a 80% to 260%, a 80% to 240%, a 80% to 220%, a 80% to 200%, a 80% to 190%, a 80% to 180%, a 80% to 170%, a 80% to 160%, a 80% to 150%, a 80% to 140%, a 80% to 130%, a 80% to 120%, a 80% to 110%, a 80% to 100%, a 80% to 95%, a 80% to 90%, a 80% to 85%, a 85% to 300%, a 85% to 280%, a 85% to 260%, a 85% to 240%, a 85% to 220%, a 85% to 200%, a 85% to 190%, a 85% to 180%, a 85% to 170%, a 85% to 160%, a 85% to 150%, a 85% to 140%, a 85% to 130%, a 85% to 120%, a 85% to 110%, a 85% to 100%, a 85% to 95%, a 85% to 90%, a 90% to 300%, a 90% to 280%, a 90% to 260%, a 90% to 240%, a 90% to 220%, a 90% to 200%, a 90% to 190%, a 90% to 180%, a 90% to 170%, a 90% to 160%, a 90% to 150%, a 90% to 140%, a 90% to 130%, a 90% to 120%, a 90% to 110%, a 90% to 100%, a 90% to 95%, a 95% to 300%, a 95% to 280%, a 95% to 260%, a 95% to 240%, a 95% to 220%, a 95% to 200%, a 95% to 190%, a 95% to 180%, a 95% to 170%, a 95% to 160%, a 95% to 150%, a 95% to 140%, a 95% to 130%, a 95% to 120%, a 95% to 110%, a 95% to 100%, a 100% to 300%, a 100% to 280%, a 100% to 260%, a 100% to 240%, a 100% to 220%, a 100% to 200%, a 100% to 190%, a 100% to 180%, a 100% to 170%, a 100% to 160%, a 100% to 150%, a 100% to 140%, a 100% to 130%, a 100% to 120%, a 100% to 110%, a 110% to 300%, a 110% to 280%, a 110% to 260%, a 110% to 240%, a 110% to 220%, a 110% to 200%, a 110% to 190%, a 110% to 180%, a 110% to 170%, a 110% to 160%, a 110% to 150%, a 110% to 140%, a 110% to 130%, a 110% to 120%, a 120% to 300%, a 120% to 280%, a 120% to 260%, a 120% to 240%, a 120% to 220%, a 120% to 200%, a 120% to 190%, a 120% to 180%, a 120% to 170%, a 120% to 160%, a 120% to 150%, a 120% to 140%, a 120% to 130%, a 130% to 300%, a 130% to 280%, a 130% to 260%, a 130% to 240%, a 130% to 220%, a 130% to 200%, a 130% to 190%, a 130% to 180%, a 130% to 170%, a 130% to 160%, a 130% to 150%, a 130% to 140%, a 140% to 300%, a 140% to 280%, a 140% to 260%, a 140% to 240%, a 140% to 220%, a 140% to 200%, a 140% to 190%, a 140% to 180%, a 140% to 170%, a 140% to 160%, a 140% to 150%, a 150% to 300%, a 150% to 280%, a 150% to 260%, a 150% to 240%, a 150% to 220%, a 150% to 200%, a 150% to 190%, a 150% to 180%, a 150% to 170%, a 150% to 160%, a 160% to 300%, a 160% to 280%, a 160% to 260%, a 160% to 240%, a 160% to 220%, a 160% to 200%, a 160% to 190%, a 160% to 180%, a 160% to 170%, a 170% to 300%, a 170% to 280%, a 170% to 260%, a 170% to 240%, a 170% to 220%, a 170% to 200%, a 170% to 190%, a 170% to 180%, a 180% to 300%, a 180% to 280%, a 180% to 260%, a 180% to 240%, a 180% to 220%, a 180% to 200%, a 180% to 190%, a 190% to 300%, a 190% to 280%, a 190% to 260%, a 190% to 240%, a 190% to 220%, a 190% to 200%, a 200% to 300%, a 200% to 280%, a 200% to 260%, a 200% to 240%, a 200% to 220%, a 220% to 300%, a 220% to 280%, a 220% to 260%, a 220% to 240%, a 240% to 300%, a 240% to 280%, a 240% to 260%, a 260% to 300%, a 260% to 280%, or a 280% to 300% increase) production of one or more cytokines (e.g., IL-2, interferon-gamma, and TNFα) as compared to a control T-cell (e.g., a T-cell including a CD28 costimulatory domain and a CD3 signaling domain) (e.g., when assessed using the same in vitro assay or in similar subjects in vivo).

In some embodiments, a population of T-cells expressing any of the single-chain chimeric antigen receptors or any of the multi-chain chimeric antigen receptors described herein can have increased (e.g., a 1% to 300%, or any of the subranges of this range described herein, increase) activation as compared a control population of T-cells (e.g., a T-cell including a CD28 costimulatory domain and a CD3 signaling domain) (e.g., when assessed using the same in vitro assay or in similar subjects in vivo).

In some embodiments, a population of T-cells expressing any of the single-chain chimeric antigen receptors or any of the multi-chain chimeric antigen receptors described herein can have increased (e.g., a 1% to 300%, or any of the subranges of this range described herein, increase) target cell killing as compared a control population of T-cells (e.g., a T-cell including a CD28 costimulatory domain and a CD3 signaling domain) (e.g., when assessed using the same in vitro assay or in similar subjects in vivo).

### Single-Chain Chimeric Antigen Receptors

### Single-Chain Chimeric Antigen Receptors Including a First Intracellular Signaling Domain, a Second Intracellular Signaling Domain, and an ITAM

Provided herein are single-chain chimeric antigen receptors that include: an extracellular antigen-binding domain (e.g., any of the antigen-binding domains described herein or known in the art); a transmembrane domain (e.g., any of the transmembrane domains described herein or known in the art); a first intracellular signaling domain from DAP-10 or DAP-12, a second intracellular signaling domain from a protein selected from the group of 4-1BB, CD27, OX40, CD40, CD28, GITR, CD2, CD5, ICAM-1, CD11a, Lck, TNFR-I, TNFR-II, FasR, CD30, ICOS, LIGHT, NKG2C, and B7-H3; and an ITAM. The single-chain chimeric antigen receptors described herein can bind to any of the exemplary antigens described herein (e.g., any of MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGI, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin) or any other antigen known in the art. In some embodiments, the single-chain chimeric antigen receptor binds specifically to a single antigen (e.g., any of the exemplary antigens described herein). In some embodiments, the single-chain chimeric antigen receptor binds specifically to two different antigens (e.g., any combination of the exemplary antigens described herein). In some embodiments, the single-chain chimeric antigen receptor or the multi-chain chimeric antigen receptor can bind specifically to a tumor antigen.

Some embodiments of these single-chain chimeric antigen receptors can include one or more (e.g., two, three, four, or five) ITAMs (e.g., any of the ITAMs described herein or known in the art). In some embodiments of these single-chain chimeric antigen receptors, the ITAM includes a cytoplasmic signaling sequence from CD3ξ (e.g., human CD3ξ.

In some embodiments of any of these single-chain chimeric antigen receptors, any two neighboring domains can be separated by 1 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, about 10 amino acids, about 8 amino acids, about 6 amino acids, about 4 amino acid, or about 3 amino acids (inclusive); about 2 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, about 10 amino acids, about 8 amino acids, about 6 amino acids, or about 4 amino acid (inclusive); about 3 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, about 10 amino acids, about 8 amino acids, about 6 amino acids, or about 5 amino acids (inclusive); about 4 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, about 10 amino acids, about 8 amino acids, or about 6 amino acids (inclusive); about 5 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, about 10 amino acids, about 8 amino acids, or about 7 amino acids (inclusive); about 6 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, about 10 amino acids, or about 8 amino acids (inclusive); about 8 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, about 15 amino acids, or about 10 amino acids (inclusive); about 10 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, about 20 amino acids, or about 15 amino acids (inclusive); about 15 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, about 25 amino acids, or about 20 amino acids (inclusive); about 20 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, about 30 amino acids, or about 25 amino acids (inclusive); about 25 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, about 35 amino acids, or about 30 amino acids (inclusive); about 30 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids, or about 35 amino acids (inclusive); about 35 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, about 45 amino acids, about 40 amino acids (inclusive); about 40 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, about 50 amino acids, or about 45 amino acids (inclusive); about 45 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, about 55 amino acids, or about 50 amino acids (inclusive); about 50 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, about 60 amino acids, or about 55 amino acids (inclusive); about 55 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, about 65 amino acids, or about 60 amino acids (inclusive); about 60 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, about 70 amino acids, or about 65 amino acids (inclusive); about 65 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, about 75 amino acids, or about 70 amino acids (inclusive); about 70 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, or about 75 amino acids (inclusive); about 75 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, about 80 amino acids, or about 75 amino acids (inclusive); about 75 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, about 90 amino acids, about 85 amino acids, or about 80 amino acids (inclusive); about 80 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, about 110 amino acids, about 100 amino acids, about 95 amino acids, or about 90 amino acids (inclusive); about 100 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, about 120 amino acids, or about 110 amino acids (inclusive); about 110 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, or about 120 amino acids (inclusive); about 120 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, about 130 amino acids, or about 120 amino acids (inclusive); about 120 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, about 140 amino acids, or about 130 amino acids (inclusive); about 130 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, about 150 amino acids, or about 140 amino acids (inclusive); about 140 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, about 160 amino acids, or about 150 amino acids (inclusive); about 150 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, about 170 amino acids, or about 160 amino acids (inclusive); about 160 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, about 180 amino acids, or about 170 amino acids (inclusive); about 170 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, about 190 amino acids, or about 180 amino acids (inclusive); about 180 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, about 200 amino acids, or about 190 amino acids (inclusive); about 190 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, about 220 amino acids, or about 200 amino acids (inclusive); about 200 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, about 240 amino acids, or about 220 amino acids (inclusive); about 220 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, about 260 amino acids, or about 240 amino acids (inclusive); about 240 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, about 280 amino acids, or about 260 amino acids (inclusive); about 260 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, about 300 amino acids, or about 280 amino acids (inclusive); about 280 amino acids to about 500 amino acids, about 480 amino acids, about 460 amino acids, about 440 amino acids, about 420 amino acids, about 400 amino acids, about 380 amino acids, about 360 amino acids, about 340 amino acids, about 320 amino acids, or about 300 amino acids (inclusive); about 300 amino acids to about 500 amino acids, about 480 amino acids, about 460 amino acids, about 440 amino acids, about 420 amino acids, about 400 amino acids, about 380 amino acids, about 360 amino acids, about 340 amino acids, or about 320 amino acids (inclusive); about 320 amino acids to about 500 amino acids, about 480 amino acids, about 460 amino acids, about 440 amino acids, about 420 amino acids, about 400 amino acids, about 380 amino acids, about 360 amino acids, or about 340 amino acids (inclusive); about 340 amino acids to about 500 amino acids, about 480 amino acids, about 460 amino acids, about 440 amino acids, about 420 amino acids, about 400 amino acids, about 380 amino acids, or about 360 amino acids (inclusive); about 360 amino acids to about 500 amino acids, about 480 amino acids, about 460 amino acids, about 440 amino acids, about 420 amino acids, about 400 amino acids, or about 380 amino acids (inclusive); about 380 amino acids to about 500 amino acids, about 480 amino acids, about 460 amino acids, about 440 amino acids, about 420 amino acids, or about 400 amino acids (inclusive); about 400 amino acids to about 500 amino acids, about 480 amino acids, about 460 amino acids, about 440 amino acids, or about 420 amino acids (inclusive); about 420 amino acids to about 500 amino acids, about 480 amino acids, about 460 amino acids, or about 440 amino acids (inclusive); about 440 amino acids to about 500 amino acids, about 480 amino acids, or about 460 amino acids (inclusive); about 460 amino acids to about 500 amino acids or about 480 amino acids (inclusive); or about 480 amino acids to about 500 amino acids (inclusive).

In some embodiments, one or more amino acids between the extracellular antigen-binding domain and the transmembrane domain is a sequence from the same endogenous single-chain polypeptide from which the transmembrane domain is derived (e.g., a CD8α hinge region, e.g., SEQ ID NO: 1). In some embodiments, a sequence comprising SEQ ID NO: 1 or 3 is positioned between the extracellular antigen-binding domain and the transmembrane domain. In some embodiments, one or more amino acids between the extracellular antigen-binding domain and the transmembrane domain is or includes a hinge region sequence of an antibody such as, without limitation, a human antibody (e.g., IgG1, IgG2, IgG3, or IgG4). In some embodiments, one or more amino acids between the extracellular antigen-binding domain and the transmembrane domain is or comprises a linker peptide sequence (e.g., a non-naturally occurring linker sequence, e.g., GS or any of the other linker sequences described herein).

(CD8 alpha hinge)
SEQ ID NO: 1
TTTPAPRPPTPAPTIASQPLSLRPEACRPAAGGAVHTRGLDFAC
(DNA sequence encoding CD8 alpha hinge of SEQ ID
NO: 1)
SEQ ID NO: 2
ACCACGACGCCAGCGCCGCGACCACCAACACCGGCGCCCACCATCGCGTC
GCAGCCCCTGTCCCTGCGCCCAGAGGCGTGCCGGCCAGCGGCGGGGGGCG
CAGTGCACACGAGGGGGCTGGACTTCGCCTGT
(Dap10 extracellular domain)
SEQ ID NO: 3
QTTPGERSSLPAFYPGTSGSCSGCGSLSLP
(Dap10 extracellular domain)
SEQ ID NO: 4
CAGACAACACCAGGCGAGAGATCTAGCCTGCCCGCCTTCTACCCTGGCAC
CAGCGGCTCTTGTTCTGGCTGTGGCAGCCTGTCTCTGCCC

In some embodiments of the single-chain chimeric antigen receptors, when going in the N-terminal to the C-terminal direction, or in the C-terminal to the N-terminal direction, the single-chain chimeric antigen receptor includes the extracellular antigen-binding domain, the transmembrane domain, the first intracellular signaling domain, the second intracellular signaling domain, and the ITAM. In some of these embodiments, one or more amino acids between the transmembrane domain and the first intracellular signaling domain is or includes a sequence from the same endogenous single chain polypeptide from which the first intracellular signaling domain or the transmembrane domain is derived. In some embodiments, one or more amino acids between the transmembrane domain and the first intracellular signaling domain is or includes a linker peptide sequence (e.g., any of the linker peptides sequences described herein or known in the art). In some of these embodiments, the transmembrane domain and the first intracellular signaling domain can directly abut each other. In some of these embodiments, one or more amino acids separating the first intracellular signaling domain and the second intracellular signaling domain is or includes a sequence from the same endogenous single chain polypeptide from which the first or second intracellular signaling domain is derived. In some embodiments, one or more amino acids between the first intracellular signaling domain and the second intracellular signaling domain is or includes a linker peptide sequence (e.g., any of the linker peptide sequences described herein or known in the art). In some of these embodiments, the first intracellular signaling domain and the second intracellular signaling domain directly abut each other. In some of these embodiments, one or more amino acids separating the second intracellular signaling domain and the ITAM is or includes a sequence from the same endogenous single chain polypeptide from which the second intracellular signaling domain or the ITAM is derived. In some embodiments, one or more amino acids between the second intracellular signaling domain and the ITAM is or includes a linker peptide sequence (e.g., any of the linker peptide sequences described herein or known in the art). In some of these embodiments, the second intracellular signaling domain and the ITAM directly abut each other.

In some embodiments of any of the single-chain chimeric antigen receptors, when going in the N-terminal to the C-terminal direction, or in the C-terminal to the N-terminal direction, include the extracellular antigen-binding domain, the transmembrane domain, the second intracellular signaling domain, the first intracellular signaling domain, and the ITAM. In some of these embodiments, one or more amino acids between the transmembrane domain and the second intracellular signaling domain is or includes a sequence from the same endogenous single chain polypeptide from which the second intracellular signaling domain or the transmembrane domain is derived. In some embodiments, one or more amino acids between the transmembrane domain and the second intracellular signaling domain is or includes a linker peptide sequence (e.g., any of the linker peptides sequences described herein or known in the art). In some of these embodiments, the transmembrane domain and the second intracellular signaling domain can directly abut each other. In some of these embodiments, one or more amino acids separating the second intracellular signaling domain and the first intracellular signaling domain is or includes a sequence from the same endogenous single chain polypeptide from which the second or first intracellular signaling domain is derived. In some embodiments, one or more amino acids between the second intracellular signaling domain and the first intracellular signaling domain is or includes a linker peptide sequence (e.g., any of the linker peptide sequences described herein or known in the art). In some of these embodiments, the second intracellular signaling domain and the first intracellular signaling domain directly abut each other. In some of these embodiments, one or more amino acids separating the first intracellular signaling domain and the ITAM is or includes a sequence from the same endogenous single chain polypeptide from which the first intracellular signaling domain or the ITAM is derived. In some embodiments, one or more amino acids between the first intracellular signaling domain and the ITAM is or includes a linker peptide sequence (e.g., any of the linker peptide sequences described herein or known in the art). In some of these embodiments, the first intracellular signaling domain and the ITAM directly abut each other.

In some embodiments of any of the single-chain chimeric antigen receptors, when going in the N-terminal to the C-terminal direction, or in the C-terminal to the N-terminal direction, include the extracellular antigen-binding domain, the transmembrane domain, the first intracellular binding domain, the ITAM, and the second intracellular signaling domain. In some of these embodiments, one or more amino acids between the transmembrane domain and the first intracellular signaling domain is or includes a sequence from the same endogenous single chain polypeptide from which the first intracellular signaling domain or the transmembrane domain is derived. In some embodiments, one or more amino acids between the transmembrane domain and the first intracellular signaling domain is or includes a linker peptide sequence (e.g., any of the linker peptides sequences described herein or known in the art). In some of these embodiments, the transmembrane domain and the first intracellular signaling domain can directly abut each other. In some of these embodiments, one or more amino acids separating the first intracellular signaling domain and the ITAM is or includes a sequence from the same endogenous single chain polypeptide from which the first intracellular signaling domain or the ITAM is derived. In some embodiments, one or more amino acids between the first intracellular signaling domain and the ITAM is or includes a linker peptide sequence (e.g., any of the linker peptide sequences described herein or known in the art). In some of these embodiments, the first intracellular signaling domain and the ITAM directly abut each other. In some of these embodiments, one or more amino acids separating the ITAM and the second intracellular signaling domain is or includes a sequence from the same endogenous single chain polypeptide from which the ITAM or the second intracellular signaling domain is derived. In some embodiments, one or more amino acids between the ITAM and the second intracellular signaling domain is or includes a linker peptide sequence (e.g., any of the linker peptide sequences described herein or known in the art). In some of these embodiments, the ITAM and the second intracellular signaling domain directly abut each other.

In some embodiments of any of the single-chain chimeric antigen receptors, when going in the N-terminal to the C-terminal direction, or in the C-terminal to the N-terminal direction, include the extracellular antigen-binding domain, the transmembrane domain, the second intracellular binding domain, the ITAM, and the first intracellular signaling domain. In some of these embodiments, one or more amino acids between the transmembrane domain and the second intracellular signaling domain is or includes a sequence from the same endogenous single chain polypeptide from which the second intracellular signaling domain or the transmembrane domain is derived. In some embodiments, one or more amino acids between the transmembrane domain and the second intracellular signaling domain is or includes a linker peptide sequence (e.g., any of the linker peptides sequences described herein or known in the art). In some of these embodiments, the transmembrane domain and the second intracellular signaling domain can directly abut each other. In some of these embodiments, one or more amino acids separating the second intracellular signaling domain and the ITAM is or includes a sequence from the same endogenous single chain polypeptide from which the second intracellular signaling domain or the ITAM is derived. In some embodiments, one or more amino acids between the second intracellular signaling domain and the ITAM is or includes a linker peptide sequence (e.g., any of the linker peptide sequences described herein or known in the art). In some of these embodiments, the second intracellular signaling domain and the ITAM directly abut each other. In some of these embodiments, one or more amino acids separating the ITAM and the first intracellular signaling domain is or includes a sequence from the same endogenous single chain polypeptide from which the ITAM or the first intracellular signaling domain is derived. In some embodiments, one or more amino acids between the ITAM and the first intracellular signaling domain is or includes a linker peptide sequence (e.g., any of the linker peptide sequences described herein or known in the art). In some of these embodiments, the ITAM and the first intracellular signaling domain directly abut each other.

In some embodiments of any of the single-chain chimeric antigen receptors, when going in the N-terminal to the C-terminal direction, or in the C-terminal to the N-terminal direction, include the extracellular antigen-binding domain, the transmembrane domain, the ITAM, the first intracellular signaling domain, and the second intracellular signaling domain. In some of these embodiments, one or more amino acids between the transmembrane domain and the ITAM is or includes a sequence from the same endogenous single chain polypeptide from which the ITAM or the transmembrane domain is derived. In some embodiments, one or more amino acids between the transmembrane domain and the ITAM is or includes a linker peptide sequence (e.g., any of the linker peptides sequences described herein or known in the art). In some of these embodiments, the transmembrane domain and the ITAM can directly abut each other. In some of these embodiments, one or more amino acids separating the ITAM and the first intracellular signaling domain is or includes a sequence from the same endogenous single chain polypeptide from which the first intracellular signaling domain or the ITAM is derived. In some embodiments, one or more amino acids between the ITAM and the first intracellular signaling domain is or includes a linker peptide sequence (e.g., any of the linker peptide sequences described herein or known in the art). In some of these embodiments, the ITAM and the first intracellular signaling domain directly abut each other. In some of these embodiments, one or more amino acids separating the first intracellular signaling domain and the second intracellular signaling domain is or includes a sequence from the same endogenous single chain polypeptide from which the first intracellular signaling domain or the second intracellular signaling domain is derived. In some embodiments, one or more amino acids between the first intracellular signaling domain and the second intracellular signaling domain is or includes a linker peptide sequence (e.g., any of the linker peptide sequences described herein or known in the art). In some of these embodiments, the first intracellular signaling domain and the second intracellular signaling domain directly abut each other.

In some embodiments of any of the single-chain chimeric antigen receptors, when going in the N-terminal to the C-terminal direction, or in the C-terminal to the N-terminal direction, include the extracellular antigen-binding domain, the transmembrane domain, the ITAM, the second intracellular signaling domain, and the first intracellular signaling domain. In some of these embodiments, one or more amino acids between the transmembrane domain and the ITAM is or includes a sequence from the same endogenous single chain polypeptide from which the ITAM or the transmembrane domain is derived. In some embodiments, one or more amino acids between the transmembrane domain and the ITAM is or includes a linker peptide sequence (e.g., any of the linker peptides sequences described herein or known in the art). In some of these embodiments, the transmembrane domain and the ITAM can directly abut each other. In some of these embodiments, one or more amino acids separating the ITAM and the second intracellular signaling domain is or includes a sequence from the same endogenous single chain polypeptide from which the second intracellular signaling domain or the ITAM is derived. In some embodiments, one or more amino acids between the ITAM and the second intracellular signaling domain is or includes a linker peptide sequence (e.g., any of the linker peptide sequences described herein or known in the art). In some of these embodiments, the ITAM and the second intracellular signaling domain directly abut each other. In some of these embodiments, one or more amino acids separating the second intracellular signaling domain and the first intracellular signaling domain is or includes a sequence from the same endogenous single chain polypeptide from which the second intracellular signaling domain or the first intracellular signaling domain is derived. In some embodiments, one or more amino acids between the second intracellular signaling domain and the first intracellular signaling domain is or includes a linker peptide sequence (e.g., any of the linker peptide sequences described herein or known in the art). In some of these embodiments, the second intracellular signaling domain and the first intracellular signaling domain directly abut each other.

Some embodiments of any of the single-chain chimeric antigen receptors described herein can further include a dimerization domain and/or a peptide tag.

### Single-Chain Chimeric Antigen Receptor Including an Intracellular Signaling Domain and an ITAM

Also provided herein are single-chain chimeric antigen receptors that include: an extracellular antigen-binding domain (e.g., any of the antigen-binding domains described herein or known in the art); a transmembrane domain (e.g., any of the transmembrane domains described herein or known in the art); an intracellular signaling domain from a protein selected from the group of 4-1BB, CD27, OX40, CD40, CD28, GITR, DAP-10, DAP-12, CD2, CD5, ICAM-1, CD11a, Lck, TNFR-I, TNFR-II, FasR, CD30, ICOS, LIGHT, NKG2C, and B7-H3; and ITAM. The single-chain chimeric antigen receptors described herein can bind to any of the exemplary antigens described herein (e.g., any of MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGI, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin) or any other antigen known in the art. In some embodiments, the single-chain chimeric antigen receptor binds specifically to a single antigen (e.g., any of the exemplary antigens described herein). In some embodiments, the single-chain chimeric antigen receptor binds specifically to two different antigens (e.g., any combination of the exemplary antigens described herein). In some embodiments, the single-chain chimeric antigen receptor or the multi-chain chimeric antigen receptor can bind specifically to a tumor antigen.

Some embodiments of these single-chain chimeric antigen receptors can include one or more (e.g., two, three, four, or five) ITAMs (e.g., any of the ITAMs described herein or known in the art). In some embodiments of these single-chain chimeric antigen receptors, the ITAM includes a cytoplasmic signaling sequence from CD3ξ (e.g., human CD3ξ).

In some embodiments of any of these single-chain chimeric antigen receptors, any two neighboring domains can be separated by 1 amino acids to about 500 amino acids (or any of the subranges of this range described herein).

In some embodiments, one or more amino acids between the extracellular antigen-binding domain and the transmembrane domain is a sequence from the same endogenous single-chain polypeptide from which the transmembrane domain is derived (e.g., a CD8α hinge region, e.g., SEQ ID NO: 1). In some embodiments, a sequence comprising SEQ ID NO: 1 or 3 is positioned between the extracellular antigen-binding domain and the transmembrane domain. In some embodiments, one or more amino acids between the extracellular antigen-binding domain and the transmembrane domain is or includes a hinge region sequence of an antibody such as, without limitation, a human antibody (e.g., IgG1, IgG2, IgG3, or IgG4). In some embodiments, one or more amino acids between the extracellular antigen-binding domain and the transmembrane domain is or comprises a linker peptide sequence (e.g., a non-naturally occurring linker sequence, e.g., GS or any of the other linker sequences described herein).

Some embodiments of the single-chain chimeric antigen receptors described herein, when going in the N-terminal to the C-terminal or in the C-termnal to the N-terminal direction, include the extracellular antigen-binding domain, the transmembrane domain, the intracellular signaling domain, and the ITAM. In some of these embodiments, one or more amino acids between the transmembrane domain and the intracellular signaling domain is or includes a sequence from the same endogenous single chain polypeptide from which the intracellular signaling domain or the transmembrane domain is derived. In some embodiments, one or more amino acids between the transmembrane domain and the intracellular signaling domain is or includes a linker peptide sequence (e.g., any of the linker peptides sequences described herein or known in the art). In some of these embodiments, the transmembrane domain and the intracellular signaling domain can directly abut each other. In some of these embodiments, one or more amino acids separating the intracellular signaling domain and the ITAM is or includes a sequence from the same endogenous single chain polypeptide from which the intracellular signaling domain or the ITAM is derived. In some embodiments, one or more amino acids between the intracellular signaling domain and the ITAM is or includes a linker peptide sequence (e.g., any of the linker peptide sequences described herein or known in the art). In some of these embodiments, the intracellular signaling domain and the ITAM directly abut each other.

Some embodiments of the single-chain chimeric antigen receptors described herein, when going in the N-terminal to the C-terminal or in the C-termnal to the N-terminal direction, include the extracellular antigen-binding domain, the transmembrane domain, the ITAM, and the intracellular signaling domain. In some of these embodiments, one or more amino acids between the transmembrane domain and the ITAM is or includes a sequence from the same endogenous single chain polypeptide from which the transmembrane domain or the ITAM is derived. In some embodiments, one or more amino acids between the transmembrane domain and the ITAM is or includes a linker peptide sequence (e.g., any of the linker peptides sequences described herein or known in the art). In some of these embodiments, the transmembrane domain and the ITAM can directly abut each other. In some of these embodiments, one or more amino acids separating the ITAM and the intracellular signaling domain is or includes a sequence from the same endogenous single chain polypeptide from which the ITAM or the intracellular signaling domain is derived. In some embodiments, one or more amino acids between the ITAM and the intracellular signaling domain is or includes a linker peptide sequence (e.g., any of the linker peptide sequences described herein or known in the art). In some of these embodiments, the ITAM and the intracellular signaling domain directly abut each other.

Some embodiments of any of the single-chain chimeric antigen receptors described herein can further include a dimerization domain and/or a peptide tag.

### Multi-Chain Chimeric Antigen Receptors

Multi-Chain Chimeric Antigen Receptors that Include at Least One First Polypeptide Including a First Intracellular Signaling Domain, a Second Intracellular Signaling Domain, and an ITAM

Also provided herein are multi-chain chimeric antigen receptors that include at least one first polypeptide including: a transmembrane domain (e.g., any of the transmembrane domains described herein or known in the art); a first intracellular binding domain from DAP-10 or DAP-12, a second intracellular signaling domain from a protein selected from the group of 4-1BB, CD27, OX40, CD40, CD28, GITR, CD2, CD5, ICAM-1, CD11a, Lck, TNFR-I, TNFR-II, FasR, CD30, ICOS, LIGHT, NKG2C, and B7-H3; and an ITAM. In some embodiments, the at least one first polypeptide can further include an antigen-binding domain (e.g., any of the antigen-binding domains described herein or known in the art). In some embodiments, the multi-chain chimeric antigen receptor further includes a second polypeptide including an antigen-binding domain (e.g., any of the antigen-binding domains described herein or known in the art) and a transmembrane domain (e.g., any of the transmembrane domains described herein). The antigen specifically bound by the antigen-binding domain in any of these multi-chain chimeric antigen receptors can be any of the antigens described herein or known in the art. In some embodiments, the multi-chain chimeric antigen receptor only binds specifically to a single antigen (e.g., any of the exemplary antigens described herein). In some embodiments, the multi-chain chimeric antigen receptor binds specifically to two different antigens (e.g., any combination of any of the exemplary antigens described herein). In some embodiments, the multi-chain chimeric antigen receptor binds to a tumor antigen.

In some embodiments of the multi-chain chimeric antigen receptors, the extracellular antigen-binding domain and the transmembrane domain in the at least one first polypeptide and/or the second polypeptide directly abut each other. In some embodiments of the multi-chain chimeric antigen receptors, 1 to about 500 amino acids (e.g., any of the subranges of this range described herein) are between the extracellular antigen-binding domain and the transmembrane domain in the at least one first polypeptide and/or the second polypeptide. In some embodiments, one or more amino acids between the extracellular antigen-binding domain and the transmembrane domain in the at least one first polypeptide and/or the second polypeptide is a sequence from the same endogenous single-chain polypeptide from which the transmembrane domain is derived. In some embodiments, one or more amino acids between the extracellular antigen-binding domain and the transmembrane domain of the at least one first polypeptide and/or the second polypeptide is or includes a hinge region sequence of human antibody (e.g., IgG1, IgG2, IgG3, or IgG4). In some embodiments, one or more amino acids between the extracellular antigen-binding domain and the transmembrane domain in the at least one first polypeptide and/or the second polypeptide is or includes a linker sequence (e.g., a non-naturally occurring linker sequence).

In some embodiments, any two neighboring domains (e.g., the transmembrane domain, the first intracellular signaling domain, the second intracellular signaling domain, and the ITAM) in the at least one first polypeptide can directly abut each other. In some embodiments, any two neighboring domains in the at least one first polypeptide (e.g., the transmembrane domain, the first intracellular signaling domain, the second intracellular signaling domain, and the ITAM) can be separated by 1 to 500 amino acids (or any subrange of this range described herein).

In some embodiments of the multi-chain chimeric antigen receptors, when going in the N-terminal to the C-terminal direction, or in the C-terminal to the N-terminal direction, the at least one first polypeptide includes the transmembrane domain, the first intracellular signaling domain, the second intracellular signaling domain, and the ITAM.

In some of these embodiments, one or more amino acids between the transmembrane domain and the first intracellular signaling domain is or includes a sequence from the same endogenous single chain polypeptide from which the first intracellular signaling domain or the transmembrane domain is derived. In some embodiments, one or more amino acids between the transmembrane domain and the first intracellular signaling domain is or includes a linker peptide sequence (e.g., any of the linker peptides sequences described herein or known in the art). In some of these embodiments, the transmembrane domain and the first intracellular signaling domain can directly abut each other. In some of these embodiments, one or more amino acids separating the first intracellular signaling domain and the second intracellular signaling domain is or includes a sequence from the same endogenous single chain polypeptide from which the first or second intracellular signaling domain is derived. In some embodiments, one or more amino acids between the first intracellular signaling domain and the second intracellular signaling domain is or includes a linker peptide sequence (e.g., any of the linker peptide sequences described herein or known in the art). In some of these embodiments, the first intracellular signaling domain and the second intracellular signaling domain directly abut each other. In some of these embodiments, one or more amino acids separating the second intracellular signaling domain and the ITAM is or includes a sequence from the same endogenous single chain polypeptide from which the second intracellular signaling domain or the ITAM is derived. In some embodiments, one or more amino acids between the second intracellular signaling domain and the ITAM is or includes a linker peptide sequence (e.g., any of the linker peptide sequences described herein or known in the art). In some of these embodiments, the second intracellular signaling domain and the ITAM directly abut each other.

In some embodiments of any of the multi-chain chimeric antigen receptors, when going in the N-terminal to the C-terminal direction, or in the C-terminal to the N-terminal direction, the at least one first polypeptide includes the transmembrane domain, the second intracellular signaling domain, the first intracellular signaling domain, and the ITAM. In some of these embodiments, one or more amino acids between the transmembrane domain and the second intracellular signaling domain is or includes a sequence from the same endogenous single chain polypeptide from which the second intracellular signaling domain or the transmembrane domain is derived. In some embodiments, one or more amino acids between the transmembrane domain and the second intracellular signaling domain is or includes a linker peptide sequence (e.g., any of the linker peptides sequences described herein or known in the art). In some of these embodiments, the transmembrane domain and the second intracellular signaling domain can directly abut each other. In some of these embodiments, one or more amino acids separating the second intracellular signaling domain and the first intracellular signaling domain is or includes a sequence from the same endogenous single chain polypeptide from which the second or first intracellular signaling domain is derived. In some embodiments, one or more amino acids between the second intracellular signaling domain and the first intracellular signaling domain is or includes a linker peptide sequence (e.g., any of the linker peptide sequences described herein or known in the art). In some of these embodiments, the second intracellular signaling domain and the first intracellular signaling domain directly abut each other. In some of these embodiments, one or more amino acids separating the first intracellular signaling domain and the ITAM is or includes a sequence from the same endogenous single chain polypeptide from which the first intracellular signaling domain or the ITAM is derived. In some embodiments, one or more amino acids between the first intracellular signaling domain and the ITAM is or includes a linker peptide sequence (e.g., any of the linker peptide sequences described herein or known in the art). In some of these embodiments, the first intracellular signaling domain and the ITAM directly abut each other.

In some embodiments of any of the multi-chain chimeric antigen receptors, when going in the N-terminal to the C-terminal direction, or in the C-terminal to the N-terminal direction, the at least one first polypeptide includes the transmembrane domain, the first intracellular binding domain, the ITAM, and the second intracellular signaling domain. In some of these embodiments, one or more amino acids between the transmembrane domain and the first intracellular signaling domain is or includes a sequence from the same endogenous single chain polypeptide from which the first intracellular signaling domain or the transmembrane domain is derived. In some embodiments, one or more amino acids between the transmembrane domain and the first intracellular signaling domain is or includes a linker peptide sequence (e.g., any of the linker peptides sequences described herein or known in the art). In some of these embodiments, the transmembrane domain and the first intracellular signaling domain can directly abut each other. In some of these embodiments, one or more amino acids separating the first intracellular signaling domain and the ITAM is or includes a sequence from the same endogenous single chain polypeptide from which the first intracellular signaling domain or the ITAM is derived. In some embodiments, one or more amino acids between the first intracellular signaling domain and the ITAM is or includes a linker peptide sequence (e.g., any of the linker peptide sequences described herein or known in the art). In some of these embodiments, the first intracellular signaling domain and the ITAM directly abut each other. In some of these embodiments, one or more amino acids separating the ITAM and the second intracellular signaling domain is or includes a sequence from the same endogenous single chain polypeptide from which the ITAM or the second intracellular signaling domain is derived. In some embodiments, one or more amino acids between the ITAM and the second intracellular signaling domain is or includes a linker peptide sequence (e.g., any of the linker peptide sequences described herein or known in the art). In some of these embodiments, the ITAM and the second intracellular signaling domain directly abut each other.

In some embodiments of any of the multi-chain chimeric antigen receptors, when going in the N-terminal to the C-terminal direction, or in the C-terminal to the N-terminal direction, the at least one first polypeptide includes the transmembrane domain, the second intracellular binding domain, the ITAM, and the first intracellular signaling domain. In some of these embodiments, one or more amino acids between the transmembrane domain and the second intracellular signaling domain is or includes a sequence from the same endogenous single chain polypeptide from which the second intracellular signaling domain or the transmembrane domain is derived. In some embodiments, one or more amino acids between the transmembrane domain and the second intracellular signaling domain is or includes a linker peptide sequence (e.g., any of the linker peptides sequences described herein or known in the art). In some of these embodiments, the transmembrane domain and the second intracellular signaling domain can directly abut each other. In some of these embodiments, one or more amino acids separating the second intracellular signaling domain and the ITAM is or includes a sequence from the same endogenous single chain polypeptide from which the second intracellular signaling domain or the ITAM is derived. In some embodiments, one or more amino acids between the second intracellular signaling domain and the ITAM is or includes a linker peptide sequence (e.g., any of the linker peptide sequences described herein or known in the art). In some of these embodiments, the second intracellular signaling domain and the ITAM directly abut each other. In some of these embodiments, one or more amino acids separating the ITAM and the first intracellular signaling domain is or includes a sequence from the same endogenous single chain polypeptide from which the ITAM or the first intracellular signaling domain is derived. In some embodiments, one or more amino acids between the ITAM and the first intracellular signaling domain is or includes a linker peptide sequence (e.g., any of the linker peptide sequences described herein or known in the art). In some of these embodiments, the ITAM and the first intracellular signaling domain directly abut each other.

In some embodiments of any of the multi-chain chimeric antigen receptors, when going in the N-terminal to the C-terminal direction, or in the C-terminal to the N-terminal direction, the at least one first polypeptide includes the transmembrane domain, the ITAM, the first intracellular signaling domain, and the second intracellular signaling domain. In some of these embodiments, one or more amino acids between the transmembrane domain and the ITAM is or includes a sequence from the same endogenous single chain polypeptide from which the ITAM or the transmembrane domain is derived. In some embodiments, one or more amino acids between the transmembrane domain and the ITAM is or includes a linker peptide sequence (e.g., any of the linker peptides sequences described herein or known in the art). In some of these embodiments, the transmembrane domain and the ITAM can directly abut each other. In some of these embodiments, one or more amino acids separating the ITAM and the first intracellular signaling domain is or includes a sequence from the same endogenous single chain polypeptide from which the first intracellular signaling domain or the ITAM is derived. In some embodiments, one or more amino acids between the ITAM and the first intracellular signaling domain is or includes a linker peptide sequence (e.g., any of the linker peptide sequences described herein or known in the art). In some of these embodiments, the ITAM and the first intracellular signaling domain directly abut each other. In some of these embodiments, one or more amino acids separating the first intracellular signaling domain and the second intracellular signaling domain is or includes a sequence from the same endogenous single chain polypeptide from which the first intracellular signaling domain or the second intracellular signaling domain is derived. In some embodiments, one or more amino acids between the first intracellular signaling domain and the second intracellular signaling domain is or includes a linker peptide sequence (e.g., any of the linker peptide sequences described herein or known in the art). In some of these embodiments, the first intracellular signaling domain and the second intracellular signaling domain directly abut each other.

In some embodiments of any of the multi-chain chimeric antigen receptors, when going in the N-terminal to the C-terminal direction, or in the C-terminal to the N-terminal direction, the at least one first polypeptide includes the transmembrane domain, the ITAM, the second intracellular signaling domain, and the first intracellular signaling domain. In some of these embodiments, one or more amino acids between the transmembrane domain and the ITAM is or includes a sequence from the same endogenous single chain polypeptide from which the ITAM or the transmembrane domain is derived. In some embodiments, one or more amino acids between the transmembrane domain and the ITAM is or includes a linker peptide sequence (e.g., any of the linker peptides sequences described herein or known in the art). In some of these embodiments, the transmembrane domain and the ITAM can directly abut each other. In some of these embodiments, one or more amino acids separating the ITAM and the second intracellular signaling domain is or includes a sequence from the same endogenous single chain polypeptide from which the second intracellular signaling domain or the ITAM is derived. In some embodiments, one or more amino acids between the ITAM and the second intracellular signaling domain is or includes a linker peptide sequence (e.g., any of the linker peptide sequences described herein or known in the art). In some of these embodiments, the ITAM and the second intracellular signaling domain directly abut each other. In some of these embodiments, one or more amino acids separating the second intracellular signaling domain and the first intracellular signaling domain is or includes a sequence from the same endogenous single chain polypeptide from which the second intracellular signaling domain or the first intracellular signaling domain is derived. In some embodiments, one or more amino acids between the second intracellular signaling domain and the first intracellular signaling domain is or includes a linker peptide sequence (e.g., any of the linker peptide sequences described herein or known in the art). In some of these embodiments, the second intracellular signaling domain and the first intracellular signaling domain directly abut each other.

In some embodiments of these multi-chain chimeric antigen receptors, the at least one first polypeptide can further include one or both of: a dimerizing domain (e.g., any dimerizing domain known in the art) and/or a peptide tag (e.g., any peptide tag known in the art).

The second polypeptide that can, e.g., include one or more (e.g., two, three, or four) of: an extracellular antigen-binding domain (e.g., any of the antigen-binding domains described herein), a transmembrane domain (e.g., any of the transmembrane domains described herein), a dimerizing domain (e.g., a dimerizing domain that can interact with a dimerizing domain in the at least one first polypeptide in a mammalian cell), one or more intracellular signaling domain (e.g., any of the intracellular signaling domains described herein), and one or more ITAM (e.g., any of the ITAMs described herein). As can be appreciated by those in the art, a pair or each pair of neighboring domains in the second polypeptide can abut each other or can be separated by 1 to about 500 amino acids (e.g., any of the subranges of this range described herein). The one or more amino acids between a pair of neighboring domains in the second polypeptide can be a sequence from an endogenous single-chain polypeptide from which a transmembrane, an intracellular signaling domain, or an ITAM present in the second polypeptide has been derived, or can be or include a linker peptide sequence. In some embodiments, any pair of neighboring domains in the second polypeptide can directly abut each other.

As can be appreciated in the art, the two or more polypeptides present in a multi-chain chimeric antigen receptor can associate via pair of domains that interact with each other (through dimerizing domains). In some embodiments, the interaction between dimerizing domains can be triggered by the addition of a small molecule. In some embodiments, the two or more polypeptides present in a multi-meric chimeric antigen receptor can associate through non-covalent interactions (e.g., between associations between dimerizing domains). In some embodiments, the two or more polypeptides present in a multi-meric chimeric antigen receptor can be through a covalent interaction (e.g., through a disulfide bond, through an ester bond, through an amide bond, through a thioester bond, or a combination thereof).

Multi-Chain Chimeric Antigen Receptors that Include at Least One First Polypeptide Including an Intracellular Signaling Domain and an ITAM

Also provided herein are multi-chain chimeric antigen receptors that include at least one first polypeptide including: a transmembrane domain (e.g., any of the transmembrane domains described herein or known in the art); an intracellular signaling domain from a protein selected from the group of 4-1BB, CD27, OX40, CD40, CD28, GITR, DAP-10, DAP-12, CD2, CD5, ICAM-1, CD11a, Lck, TNFR-I, TNFR-II, FasR, CD30, ICOS, LIGHT, NKG2C, and B7-H3; and an ITAM. In some embodiments, the at least one first polypeptide can further include an antigen-binding domain (e.g., any of the antigen-binding domains described herein or known in the art). In some embodiments, the multi-chain chimeric antigen receptor further includes a second polypeptide including an antigen-binding domain (e.g., any of the antigen-binding domains described herein or known in the art) and a transmembrane domain (e.g., any of the transmembrane domains described herein). The antigen specifically bound by the antigen-binding domain in any of these multi-chain chimeric antigen receptors can be any of the antigens described herein or known in the art. In some embodiments, the multi-chain chimeric antigen receptor only binds specifically to a single antigen (e.g., any of the exemplary antigens described herein). In some embodiments, the multi-chain chimeric antigen receptor binds specifically to two different antigens (e.g., any combination of any of the exemplary antigens described herein). In some embodiments, the multi-chain chimeric antigen receptor binds to a tumor antigen.

In some embodiments of the multi-chain chimeric antigen receptors, the extracellular antigen-binding domain and the transmembrane domain in the at least one first polypeptide and/or the second polypeptide directly abut each other. In some embodiments of the multi-chain chimeric antigen receptors, 1 to about 500 amino acids (e.g., any of the subranges of this range described herein) are between the extracellular antigen-binding domain and the transmembrane domain in the at least one first polypeptide and/or the second polypeptide. In some embodiments, one or more amino acids between the extracellular antigen-binding domain and the transmembrane domain in the at least one first polypeptide and/or the second polypeptide is a sequence from the same endogenous single-chain polypeptide from which the transmembrane domain is derived. In some embodiments, one or more amino acids between the extracellular antigen-binding domain and the transmembrane domain of the at least one first polypeptide and/or the second polypeptide is or includes a hinge region sequence of human antibody (e.g., IgG1, IgG2, IgG3, or IgG4). In some embodiments, one or more amino acids between the extracellular antigen-binding domain and the transmembrane domain in the at least one first polypeptide and/or the second polypeptide is or includes a linker sequence (e.g., a non-naturally occurring linker sequence).

In some embodiments, any two neighboring domains (e.g., the transmembrane domain, the intracellular signaling domain, and the ITAM) in the at least one first polypeptide can directly abut each other. In some embodiments, any two neighboring domains in the at least one first polypeptide (e.g., the transmembrane domain, the intracellular signaling domain, and the ITAM) can be separated by 1 to 500 amino acids (or any subrange of this range described herein).

Some embodiments of the multi-chain chimeric antigen receptors described herein, when going in the N-terminal to the C-terminal or in the C-termnal to the N-terminal direction, the at least one first polypeptide includes the transmembrane domain, the intracellular signaling domain, and the ITAM. In some of these embodiments, one or more amino acids between the transmembrane domain and the intracellular signaling domain is or includes a sequence from the same endogenous single chain polypeptide from which the intracellular signaling domain or the transmembrane domain is derived. In some embodiments, one or more amino acids between the transmembrane domain and the intracellular signaling domain is or includes a linker peptide sequence (e.g., any of the linker peptides sequences described herein or known in the art). In some of these embodiments, the transmembrane domain and the intracellular signaling domain can directly abut each other. In some of these embodiments, one or more amino acids separating the intracellular signaling domain and the ITAM is or includes a sequence from the same endogenous single chain polypeptide from which the intracellular signaling domain or the ITAM is derived. In some embodiments, one or more amino acids between the intracellular signaling domain and the ITAM is or includes a linker peptide sequence (e.g., any of the linker peptide sequences described herein or known in the art). In some of these embodiments, the intracellular signaling domain and the ITAM directly abut each other.

Some embodiments of the multi-chain chimeric antigen receptors described herein, when going in the N-terminal to the C-terminal or in the C-termnal to the N-terminal direction, the at least one first polypeptide includes the transmembrane domain, the ITAM, and the intracellular signaling domain. In some of these embodiments, one or more amino acids between the transmembrane domain and the ITAM is or includes a sequence from the same endogenous single chain polypeptide from which the transmembrane domain or the ITAM is derived. In some embodiments, one or more amino acids between the transmembrane domain and the ITAM is or includes a linker peptide sequence (e.g., any of the linker peptides sequences described herein or known in the art). In some of these embodiments, the transmembrane domain and the ITAM can directly abut each other. In some of these embodiments, one or more amino acids separating the ITAM and the intracellular signaling domain is or includes a sequence from the same endogenous single chain polypeptide from which the ITAM or the intracellular signaling domain is derived. In some embodiments, one or more amino acids between the ITAM and the intracellular signaling domain is or includes a linker peptide sequence (e.g., any of the linker peptide sequences described herein or known in the art). In some of these embodiments, the ITAM and the intracellular signaling domain directly abut each other.

In some embodiments of these multi-chain chimeric antigen receptors, the at least one first polypeptide can further include one or both of: a dimerizing domain (e.g., any dimerizing domain known in the art) and/or a peptide tag (e.g., any peptide tag known in the art).

The second polypeptide that can, e.g., include one or more (e.g., two, three, or four) of: an extracellular antigen-binding domain (e.g., any of the antigen-binding domains described herein), a transmembrane domain (e.g., any of the transmembrane domains described herein), a dimerizing domain (e.g., a dimerizing domain that can interact with a dimerizing domain in the at least one first polypeptide in a mammalian cell), one or more intracellular signaling domain (e.g., any of the intracellular signaling domains described herein), and one or more ITAM (e.g., any of the ITAMs described herein). As can be appreciated by those in the art, a pair or each pair of neighboring domains in the second polypeptide can abut each other or can be separated by 1 to about 500 amino acids (e.g., any of the subranges of this range described herein). The one or more amino acids between a pair of neighboring domains in the second polypeptide can be a sequence from an endogenous single-chain polypeptide from which a transmembrane, an intracellular signaling domain, or an ITAM present in the second polypeptide has been derived, or can be or include a linker peptide sequence. In some embodiments, any pair of neighboring domains in the second polypeptide can directly abut each other.

As can be appreciated in the art, the two or more polypeptides present in a multi-chain chimeric antigen receptor can associate via pair of domains that interact with each other (through dimerizing domains). In some embodiments, the interaction between dimerizing domains can be triggered by the addition of a small molecule. In some embodiments, the two or more polypeptides present in a multi-meric chimeric antigen receptor can associate through non-covalent interactions (e.g., between associations between dimerizing domains). In some embodiments, the two or more polypeptides present in a multi-meric chimeric antigen receptor can be through a covalent interaction (e.g., through a disulfide bond, through an ester bond, through an amide bond, through a thioester bond, or a combination thereof).

### Transmembrane Domains

In some embodiments, a single-chain chimeric antigen receptor or a multi-chain chimeric antigen receptor includes a transmembrane domain, or portion thereof, from an endogenous polypeptide, where the endogenous polypeptide is selected from the group of: an α chain of a T cell receptor, a β chain of the T cell receptor, a ξ chain of the T cell receptor, CD28 (also known as Tp44), CD3ε, CD3δ, CD3γ, CD33, CD37 (also known as GP52-40 or TSPAN26), CD64 (also known as FCGR1A), CD80 (also known as B7, B7-1, B7.1, BB1, CD28LG, CD28LG1, and LAB7), CD45 (also known as PTPRC, B220, CD45R, GP180, L-CA, LCA, LY5, T200, and protein tyrosine phosphatase, receptor type C), CD4, CD5 (also known as LEU1 and T1), CD8α (also known as Leu2, MAL, and p32), CD9 (also known as BTCC-1, DRAP-27, MIC3, MRP-1, TSPAN-29, and TSPAN29), CD16 (also known as FCGR3 andFCG3), CD22 (also known as SIGLEC-2 and SIGLEC2), CD86 (also known as B7-2, B7.2, B70, CD28LG2, and LAB72), CD134 (also known as TNFRSF4, ACT35, RP5-902P8.3, IMD16, OX40, TXGPIL, and tumor necrosis factor receptor superfamily member 4), CD137 (also known as TNFRSF9, 4-1BB, CDw137, ILA, and tumor necrosis factor receptor superfamily member 9), CD27 (also known as S152, S152.LPFS2, T14, TNFRSF7, and Tp55), CD152 (also known as CTLA4, ALPS5, CELIAC3, CTLA-4, GRD4, GSE, IDDM12, and cytotoxic T-lymphocyte associated protein 4), PD1 (also known as PDCD1, CD279, PD-1, SLEB2, hPD-1, hPD-1, hSLE1, and Programmed cell death 1), ICOS (also known as AILIM, CD278, and CVID1), CD272 (also known as BTLA and BTLA1), CD30 (also known as TNFRSF8, DIS166E, and Ki-1), GITR (also known as TNFRSF18, RP5-902P8.2, AITR, CD357, and GITR-D), HVEM (also known as TNFRSF14, RP3-395M20.6, ATAR, CD270, HVEA, HVEM, LIGHTR, and TR2), DAP10, and CD154 (also known as CD40LG, CD40L, HIGM1, IGM, IMD3, T-BAM, TNFSF5, TRAP, gp39, hCD40L, and CD40 ligand). The letters “CD” is the previous sentence stand for “Cluster of Differentiation.” E.g., CD3 stands for “Cluster of Differentiation 3.” In some embodiments, a single-chain chimeric antigen receptor or a multi-chain chimeric antigen receptor includes a transmembrane domain, or portion thereof, from an endogenous mammalian (e.g., human) polypeptide (e.g., a mammalian or human homolog of any of the polypeptides listed above).

Any transmembrane domain, or portion thereof, that serves to anchor an endogenous polypeptide in a lipid bilayer (e.g., plasma membrane) of a mammalian cell is suitable for use in accordance with compositions and methods disclosed herein. In some embodiments, a single-chain chimeric antigen receptor or a multi-chain chimeric antigen receptor includes a transmembrane domain, or portion thereof, from human CD28, e.g., Accession No. P01747, e.g., amino acids 153 to 179 of SEQ ID NO: 5. In some embodiments, a single-chain chimeric antigen receptor or a multi-chain chimeric antigen receptor includes a transmembrane domain that is at least 80% (e.g., at least 82%, at least 84%, at least 85%, at least 86%, at least 88%, at least 90%, at least 92%, at least 94%, at least 95%, at least 96%, at least 98%, or at least 99% identical) to amino acids 153 to 179 of SEQ ID NO: 5, or a portion thereof.

(Transmembrane domain of human CD28)
SEQ ID NO: 5
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In some embodiments, transmembrane domain can comprise a sequence at least 80% (e.g., at least 82%, at least 84%, at least 86%, at least 88%, at least 90%, at least 92%, at least 94%, at least 96%, at least 98%, at least 99%, or 100% identical) to SEQ ID NO: 6 (or a portion thereof).

(CD8 alpha transmembrane domain)
SEQ ID NO: 6
DIYIWAPLAGTCGVLLLSLVITLYC

In some embodiments, a single-chain chimeric antigen receptor or a multi-chain chimeric antigen receptor includes a transmembrane domain, or portion thereof, from human CD3, e.g., Accession No. P20963, e.g., amino acids 31 to 51 of SEQ ID NO: 7. In some embodiments, a single-chain chimeric antigen receptor or a multi-chain chimeric antigen receptor includes a transmembrane domain that is or includes a sequence that is at least 80% (e.g., at least 82%, at least 84%, at least 85%, at least 86%, at least 88%, at least 90%, at least 92%, at least 94%, at least 95%, at least 96%, at least 98%, or at least 99% identical) to amino acids 31 to 51 of SEQ ID NO: 7.

(Transmembrane domain of human CD3)
SEQ ID NO: 7
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In some embodiments, a single-chain chimeric antigen receptor or a multi-chain chimeric antigen receptor includes a transmembrane domain, or portion thereof, of any one of SEQ ID Nos. 8-14:

(SEQ ID NO: 8)

LGLLVAGVLVLLVSLGVAIHLCC;

(SEQ ID NO: 9)

VAAILGLGLVLGLLGPLAILLALYLL;

(SEQ ID NO: 10)

ALIVLGGVAGLLLFIGLGIFFCVRC;

(SEQ ID NO: 11)

LCYLLDGILFIYGVILTALFLRV;

(SEQ ID NO: 12)

WVLVVVGGVLACYSLLVTVAFIIFWV;

(SEQ ID NO: 13)

IYIWAPLAGTCGVLLLSLVITLYC;
and

(SEQ ID NO: 14)

ALPAALAVISFLLGLGLGVACVLA.

In some embodiments, a single-chain chimeric antigen receptor or a multi-chain chimeric antigen receptor includes a transmembrane domain that is or includes a sequence that is at least 80% (e.g., at least 82%, at least 84%, at least 85%, at least 86%, at least 88%, at least 90%, at least 92%, at least 94%, at least 95%, at least 96%, at least 98%, or at least 99% identical) to any one of SEQ ID Nos. 8-14.

In some embodiments, a single-chain chimeric antigen receptor or a multi-chain chimeric antigen receptor includes a transmembrane domain that is or includes a sequence that is at least 80% (e.g., at least 82%, at least 84%, at least 85%, at least 86%, at least 88%, at least 90%, at least 92%, at least 94%, at least 95%, at least 96%, at least 98%, or at least 99% identical, or 100% identical) to SEQ ID NO: 13.

(CD8 transmembrane domain)
SEQ ID NO: 13
IYIWAPLAGTCGVLLLSLVITLYC
(DNA sequence encoding CD8 transmembrane domain)
SEQ ID NO: 15
atctatatttgggcacccctggctggaacctgcggagtgctgctgctgtc
tctcgtgattacactgtattgc
(DNA sequence encoding CD8 alpha transmembrane
domain)
SEQ ID NO: 16
gatatctacatctgggcgcccttggccgggacttgtggggtccttctcct
gtcactggttatcaccctttactgc

As will be appreciated by those of ordinary skill in the art, certain endogenous polypeptides have two or more isoforms that differ at least in their primary polypeptide sequence. A single-chain chimeric antigen receptor or a multi-chain chimeric antigen receptor disclosed herein can include a transmembrane domain that includes a sequence of amino acids from any isoform of an endogenous transmembrane protein (e.g., an endogenous mammalian, e.g., human, transmembrane protein) including, e.g., an isoform (e.g., a human isoform) of: an α chain of a T cell receptor, a β chain of the T cell receptor, a ξ chain of the T cell receptor, CD28, CD3ε, CD3δ, CD3γ, CD33, CD37, CD64, CD80, CD45, CD4, CD5, CD8α, CD9, CD16, CD22, CD86, CD27, CD152, PD1, or CD154.

In some embodiments, a transmembrane domain, or portion thereof, of a single-chain chimeric antigen receptor or a multi-chain chimeric antigen receptor includes a sequence of amino acids that exhibits at least 85%, 86%, 87%, 88%, 89%, 90%, 91%, 92%, 93%, 94%, 95%, 96%, 97%, 98%, 99% or more sequence identity to the transmembrane domains from one or more of the following endogenous mammalian (e.g., human) transmembrane proteins: an α chain of a T cell receptor, a β chain of the T cell receptor, a ξ chain of the T cell receptor, CD28, CD3ε, CD3δ, CD37, CD33, CD37, CD64, CD80, CD45, CD4, CD5, CD8α, CD9, CD16, CD22, CD86, CD27, CD152, PD1, or CD154. In some embodiments, a transmembrane domain, or portion thereof, of a single-chain chimeric antigen receptor or a multi-chain chimeric antigen receptor includes a sequence of amino acids having one or more amino acid substitutions, deletions, or additions as compared to the transmembrane domain of an endogenous mammalian (e.g., human) transmembrane protein: an α chain of a T cell receptor, a β chain of the T cell receptor, a ξ chain of the T cell receptor, CD28, CD3ε, CD3δ, CD3γ, CD33, CD37, CD64, CD80, CD45, CD4, CD5, CD8α, CD9, CD16, CD22, CD86, CD27, CD152, PD1, or CD154.

In some embodiments, a single-chain chimeric antigen receptor or a multi-chain chimeric antigen receptor includes a synthetic transmembrane domain. In some cases, a synthetic transmembrane domain can include predominantly hydrophobic residues such as, without limitation, leucine and valine. In some embodiments, a synthetic transmembrane domain includes a triplet of phenylalanine, tryptophan, and valine at each end of the domain.

In some embodiments, a single-chain chimeric antigen receptor or a multi-chain chimeric antigen receptor includes a transmembrane domain that is a chimeric transmembrane domain having portions of a transmembrane domain from two or more endogenous mammalian (e.g., human) transmembrane polypeptides such as, without limitation, an α chain of a T cell receptor, a β chain of the T cell receptor, a ξ chain of the T cell receptor, CD28, CD3ε, CD3δ, CD3γ, CD33, CD37, CD64, CD80, CD45, CD4, CD5, CD8α, CD9, CD16, CD22, CD86, CD27, CD152, PD1, and CD154, such that the two or more portions of transmembrane domains together constitute a functional transmembrane domain. In some embodiments, such a portion of a chimeric transmembrane domain can include one or more amino acid substitutions, deletions, or additions as compared to a corresponding portion of a wild type transmembrane domain.

A transmembrane domain can include one, two, three, four, five, six, seven, eight, nine, or ten contiguous amino acid sequences that each traverse a lipid bilayer when present in the corresponding endogenous polypeptide when expressed in a mammalian cell. As is known in the art, a transmembrane domain can, e.g., include at least one (e.g., two, three, four, five, six, seven, eight, nine, or ten) contiguous amino acid sequence (that traverses a lipid bilayer when present in the corresponding endogenous polypeptide when expressed in a mammalian cell) that has α-helical secondary structure in the lipid bilayer. In some embodiments, a transmembrane domain can include two or more contiguous amino acid sequences (that each traverse a lipid bilayer when present in the corresponding endogenous polypeptide when expressed in a mammalian cell) that form a β-barrel secondary structure in the lipid bilayer. Additional examples and features of transmembrane domains are known in the art.

### Antigen-Binding Domains

In some embodiments of the single-chain chimeric antigen receptors or the multi-chain chimeric antigen receptors, the antigen-binding domain can be selected from a scFv, a scFv-Fc, a VHH domain, a V_{NAR} domain, a (scFv)₂, and a BiTE. Additional examples of antigen-binding domains that can be used in a single-chain chimeric antigen receptor or a multi-chain chimeric antigen receptor are known in the art.

A single-chain Fv or scFv fragment includes a V_{H} domain and a V_{L} domain in a single polypeptide chain. The V_{H} and V_{L} are generally linked by a peptide linker. In other examples, the linker can be a single amino acid. In some examples, the linker can be a chemical bond. See, e.g., Pluckthun, Antibodies from E. coli. In Rosenberg M. & Moore G. P. (Eds.), The Pharmacology of Monoclonal Antibodies, Vol. 113, pp. 269-315, Spinger-Verlag, New York, 1994. An exemplary V_{L} and V_{H} sequences that can be used in a scFv are shown in SEQ ID NOs: 18 and 21. An exemplary linker that can be used between the V_{L} and V_{H} domains in an scFv can be any of the exemplary linkers described herein (e.g., a linker having the sequence of SEQ ID NO: 31).

Sc-Fv-Fc fragments include an scFv attached to an Fc domain. For example, an Fc domain can be attached, e.g., to the C-terminus of the scFv. The Fc domain can follow the V_{L} or V_{H}, depending on the orientation of the variable domains in the scFv. The Fc domain can be any Fc domain known in the art. In some examples, the Fc domain is an IgG1, IgG2, IgG3, or IgG4 Fc domain (e.g., a human IgG1, IgG2, IgG3, or IgG4 Fc domain).

BiTEs are antigen-binding domains that include two V_{L} and two V_{H} in a single polypeptide that together form two scFvs, which can each bind to different epitopes on the same antigen or each bind to different antigens. See, e.g., Baeuerle et al., Curr. Opin. Mol. Ther. 11:22-30, 2009; Wolf et al., Drug Discovery Today 10:1237-1244, 2005; and Huehls et al., Immunol. Cell Biol. 93:290-296, 2015.

A V_{H}H domain is a single monomeric variable antibody domain found in camelids, and a V_{NAR} domain is a single monomeric variable antibody domain found in cartilaginous fish. V_{H}H domains and V_{NAR} domains are described in, e.g., Van Audenhove et al., EBioMedicine 8:40-48, 2016; Krah et al., Immunopharmacol. Immunotoxicol. 38:21-28, 2016; Cromie et al., Curr. Top. Med. Chem. 15:2543-2557, 2016; Kijanka et al., Nanomedicine 10:161-174, 2015; Kovaleva et al., Expert. Opin. Biol. Ther. 14:1527-1539, 2014; De Meyer et al., Trends Biotechnol. 32:263-270, 2014; Mujic-Delic et al., Trends Pharmacol. Sci. 35:247-255, 2014; Muyldermans, Ann. Rev. Biochem. 82:775-797, 2013; Vincke et al., Methods Mol. Biol. 911:15-26, 2012; Rahbarizadeh et al., Immunol. Invest. 40:299-338, 2011; Van Bockstaele et al., Curr. Opin. Investig. Drugs 10:1212-1224, 2009; Wesolowski et al., Med. Microbiol. Immunol. 198:157-174, 2009; De Genst et al., Dev. Comp. Immunol. 30:187-198, 2006; Muyldermans, J. Biotechnol. 74:277-302, 2001; and Muyldermans et al., Trends Biochem. Sci. 26:230-235, 2001.

In some embodiments of the multi-chain chimeric antigen receptors, at least two of the polypeptides that make up the multi-chain chimeric antigen receptor can interact together to form an antigen-binding domain. In such embodiments, the antigen-binding domain can be selected from the group of an antigen-binding antibody fragment, a dual-affinity re-targeting antibody (DART), Fab-scFv-Fc, a triomab, a crossmab, an ortho-Fab IgG, IgG-scFv, scFv2-Fc, a bi-nanobody, tanden antibody, a DART-Fc, a scFv-HAS-scFv, DNL-Fab3, DAF (two-in-one or four-in-one), DutaMab, DT-IgG, knobs-in-holes common LC, knobs-in-holes assembly, Fab-arm exchange antibody, SEEDbody, Triomab, LUZ-Y, scDiabody-Fc, Fcab, ka-body, orthogonal Fab, DVD-IgG, IgG (H)-scFv, scFv-(H) IgG, IgG(L)-scFv, scFv-(L)-IgG, IgG (L,H)-Fc, IgG (H)-V, V(H)-IgG, IgG(L)-V, V(L)-IgG, KIH IgG-scFab, 2scFv-IgG, IgG-2scFv, scFv4-Ig, Zybody, DVI-IgG, nanobody, nanobody-HSA, a diabody, a TandAb, scDiabody, scDiabody-CH3, charge pair antibody, diabody-CH3, Cov-X-Bod, Triple Body, miniantibody, a DVD-Ig, minibody, a 2-in-1-IgG TriBi minibody, scFv-CH3 KIH, Fab-scFv, scFv-CH-CL-scFv, F(ab′)₂-scFV2, scFv-KIH, tetravalent HCAb, diabody-Fc, tandem scFv-Fc, intrabody, dock and lock bispecific antibody, ImmTAC, HSAbody, scDiabody-HAS, tandem scFv, IgG-IgG, and scFv1-PEG-scFv2.

Non-limiting examples of an antigen-binding antibody fragments include an Fv fragment, a Fab fragment, a F(ab′)₂ fragment, and a Fab′ fragment. Additional examples of an antigen-binding antibody fragment is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgAQ1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

Examples of DVD-Igs are described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645. Examples of DARTs are described in, e.g., Garber, Nature Reviews Drug Discovery 13:799-801, 2014. Examples of triomabs, kih IgG with a common LCs, crossmabs, ortho-Fab IgGs, 2-in-1-IgGs, IgG-ScFvs, scFv2-Fcs, bi-nanobodies, tanden antibodies, DART-Fcs, scFv-HAS-scFvs, and DNL-Fab3s are described in, e.g., Kontermann et al., Drug Discovery Today 20:838-847, 2015. Examples of DAFs (two-in-one or four-in-one), DutaMabs, DT-IgGs, knobs-in-holes common LCs, knobs-in-holes assemblies, charge pair antibodies, Fab-arm exchange antibodies, SEEDbodies, Triomabs, LUZ-Ys, Fcabs, kλ-bodies, orthogonal Fabs, DVD-IgGs, IgG (H)-scFvs, scFv-(H) IgGs, IgG(L)-scFvs, scFv-(L)-IgGs, IgG (L,H)-Fcs, IgG (H)-Vs, V(H)-IgGs, IgG(L)-Vs, V(L)-IgGs, KIH IgG-scFabs, 2scFv-IgGs, IgG-2scFvs, scFv4-Igs, Zybodies, DVI-IgGs, nanobodies, nanobody-HSAs, diabodies, TandAbs, scDiabodies, scDiabody-CH3s, Diabody-CH3s, Triple Bodies, miniantibodies, minibodies, TriBi minibodies, scFv-CH3 KIHs, Fab-scFvs, scFv-CH-CL-scFvs, F(ab′)₂-scFV2s, scFv-KIHs, Fab-scFv-Fcs, tetravalent HCAbs, scDiabody-Fcs, diabody-Fcs, tandem scFv-Fcs, intrabodies, dock and lock bispecific antibodies, ImmTACs, HSAbodies, scDiabody-HASs, tandem scFvs, IgG-IgGs, Cov-X-Bodies, and scFv1-PEG-scFv2s are described in, e.g., Spiess et al., Mol. Immunol. 67:95-106, 2015.

Exemplary sequences that encode an anti-CD19 scFv are shown below.

(FMC63 V_{L})
SEQ ID NO: 17
gacatccagatgacccagaccaccagcagcctgagcgccagcctgggcga
tagagtgaccatcagctgcagagccagccaggacatcagcaagtacctga
actggtatcagcagaaacccgacggcaccgtgaagctgctgatctaccac
accagcagactgcacagcggcgtgcccagcagattttctggcagcggctc
cggcaccgactacagcctgaccatctccaacctggaacaggaagatatcg
ctacctacttctgtcagcaaggcaacaccctgccctacaccttcggcgga
ggcaccaagctggaaatcaca
(FMC63 V_{L})
SEQ ID NO: 18
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEIT
((G₄S)₃ linker between V_{L} and V_{H})
SEQ ID NO: 19
ggcggcggaggatctggcggaggcggaagtggcggagggggatct
(FMC63 V_{H})
SEQ ID NO: 20
gaagtgaaactgcaggaaagcggccctggcctggtggccccatctcagtc
tctgagcgtgacctgtaccgtgtccggcgtgtccctgcctgactatggcg
tgtcctggatcagacagccccccagaaagggcctggaatggctgggagtg
atctggggcagcgagacaacctactacaacagcgccctgaagtcccggct
gaccatcatcaaggacaactccaagagccaggtgttcctgaagatgaaca
gcctgcagaccgacgacaccgccatctactactgcgccaagcactactac
tacggcggcagctacgccatggactactggggccagggcacaagcgtgac
cgtgtctagc
(FMC63 V_{H})
SEQ ID NO: 21
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSS

Any of the antigen-binding domains described herein can bind to an antigen with a dissociation equilibrium constant (K_{D}) of less than 1×10⁻⁷ M, less than 1×10⁻⁸ M, less than 1×10⁻⁹ M, less than 1×10⁻¹⁰M, less than 1×10⁻¹¹ M, less than 1×10⁻¹² M, or less than 1×10⁻¹³ M. In some embodiments, the antigen-binding protein complexes provided herein can bind to a first and/or second antigen with a K_{D} of about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, or about 1×10⁻⁹ M to about 1×10⁻¹¹ M (inclusive). A variety of different methods known in the art can be used to determine the K_{D} value of an antigen-binding domain (e.g., an electrophoretic mobility shift assay, a filter binding assay, surface plasmon resonance, and a biomolecular binding kinetics assay, etc.).

### Linker Peptide Sequences

In some embodiments, any two domains of single-chain chimeric antigen receptor, or any two neighboring domains within of a single-chain polypeptide that makes up a multi-chain chimeric antigen receptor (e.g., the at least one first polypeptide and second polypeptide) can be separated by a sequence that includes one or more (e.g., one, two, three, four, or five) linker peptide sequences. The length of a linker peptide sequence can be about 2 amino acids to about 100 amino acids, about 2 amino acids to about 95 amino acids, about 2 amino acids to about 90 amino acids, about 2 amino acids to about 85 amino acids, about 2 amino acids to about 80 amino acids, about 2 amino acids to about 75 amino acids, about 2 amino acids to about 70 amino acids, about 2 amino acids to about 65 amino acids, about 2 amino acids to about 60 amino acids, about 2 amino acids to about 55 amino acids, about 2 amino acids to about 50 amino acids, about 2 amino acids to about 45 amino acids, about 2 amino acids to about 40 amino acids, about 2 amino acids to about 35 amino acids, about 2 amino acids to about 30 amino acids, about 2 amino acids to about 25 amino acids, about 2 amino acids to about 20 amino acids, about 2 amino acids to about 15 amino acids, about 2 amino acids to about 14 amino acids, about 2 amino acids to about 12 amino acids, about 2 amino acids to about 10 amino acids, about 2 amino acids to about 8 amino acids, about 2 amino acids to about 6 amino acids, about 2 amino acids to about 4 amino acids, about 4 amino acids to about 100 amino acids, about 4 amino acids to about 95 amino acids, about 4 amino acids to about 90 amino acids, about 4 amino acids to about 85 amino acids, about 4 amino acids to about 80 amino acids, about 4 amino acids to about 75 amino acids, about 4 amino acids to about 70 amino acids, about 4 amino acids to about 65 amino acids, about 4 amino acids to about 60 amino acids, about 4 amino acids to about 55 amino acids, about 4 amino acids to about 50 amino acids, about 4 amino acids to about 45 amino acids, about 4 amino acids to about 40 amino acids, about 4 amino acids to about 35 amino acids, about 4 amino acids to about 30 amino acids, about 4 amino acids to about 25 amino acids, about 4 amino acids to about 20 amino acids, about 4 amino acids to about 15 amino acids, about 4 amino acids to about 14 amino acids, about 4 amino acids to about 12 amino acids, about 4 amino acids to about 10 amino acids, about 4 amino acids to about 8 amino acids, about 4 amino acids to about 6 amino acids, about 6 amino acids to about 100 amino acids, about 6 amino acids to about 95 amino acids, about 6 amino acids to about 90 amino acids, about 6 amino acids to about 85 amino acids, about 6 amino acids to about 80 amino acids, about 6 amino acids to about 75 amino acids, about 6 amino acids to about 70 amino acids, about 6 amino acids to about 65 amino acids, about 6 amino acids to about 60 amino acids, about 6 amino acids to about 55 amino acids, about 6 amino acids to about 50 amino acids, about 6 amino acids to about 45 amino acids, about 6 amino acids to about 40 amino acids, about 6 amino acids to about 35 amino acids, about 6 amino acids to about 30 amino acids, about 6 amino acids to about 25 amino acids, about 6 amino acids to about 20 amino acids, about 6 amino acids to about 15 amino acids, about 6 amino acids to about 14 amino acids, about 6 amino acids to about 12 amino acids, about 6 amino acids to about 10 amino acids, about 6 amino acids to about 8 amino acids, about 8 amino acids to about 100 amino acids, about 8 amino acids to about 95 amino acids, about 8 amino acids to about 90 amino acids, about 8 amino acids to about 85 amino acids, about 8 amino acids to about 80 amino acids, about 8 amino acids to about 75 amino acids, about 8 amino acids to about 70 amino acids, about 8 amino acids to about 65 amino acids, about 8 amino acids to about 60 amino acids, about 8 amino acids to about 55 amino acids, about 8 amino acids to about 50 amino acids, about 8 amino acids to about 45 amino acids, about 8 amino acids to about 40 amino acids, about 8 amino acids to about 35 amino acids, about 8 amino acids to about 30 amino acids, about 8 amino acids to about 25 amino acids, about 8 amino acids to about 20 amino acids, about 8 amino acids to about 15 amino acids, about 8 amino acids to about 14 amino acids, about 8 amino acids to about 12 amino acids, about 8 amino acids to about 10 amino acids, about 10 amino acids to about 100 amino acids, about 10 amino acids to about 95 amino acids, about 10 amino acids to about 90 amino acids, about 10 amino acids to about 85 amino acids, about 10 amino acids to about 80 amino acids, about 10 amino acids to about 75 amino acids, about 10 amino acids to about 70 amino acids, about 10 amino acids to about 65 amino acids, about 10 amino acids to about 60 amino acids, about 10 amino acids to about 55 amino acids, about 10 amino acids to about 50 amino acids, about 10 amino acids to about 45 amino acids, about 10 amino acids to about 40 amino acids, about 10 amino acids to about 35 amino acids, about 10 amino acids to about 30 amino acids, about 10 amino acids to about 25 amino acids, about 10 amino acids to about 20 amino acids, about 10 amino acids to about 15 amino acids, about 10 amino acids to about 14 amino acids, about 10 amino acids to about 12 amino acids, about 12 amino acids to about 100 amino acids, about 12 amino acids to about 95 amino acids, about 12 amino acids to about 90 amino acids, about 12 amino acids to about 85 amino acids, about 12 amino acids to about 80 amino acids, about 12 amino acids to about 75 amino acids, about 12 amino acids to about 70 amino acids, about 12 amino acids to about 65 amino acids, about 12 amino acids to about 60 amino acids, about 12 amino acids to about 55 amino acids, about 12 amino acids to about 50 amino acids, about 12 amino acids to about 45 amino acids, about 12 amino acids to about 40 amino acids, about 12 amino acids to about 35 amino acids, about 12 amino acids to about 30 amino acids, about 12 amino acids to about 25 amino acids, about 12 amino acids to about 20 amino acids, about 12 amino acids to about 15 amino acids, about 12 amino acids to about 14 amino acids, about 15 amino acids to about 100 amino acids, about 15 amino acids to about 95 amino acids, about 15 amino acids to about 90 amino acids, about 15 amino acids to about 85 amino acids, about 15 amino acids to about 80 amino acids, about 15 amino acids to about 75 amino acids, about 15 amino acids to about 70 amino acids, about 15 amino acids to about 65 amino acids, about 15 amino acids to about 60 amino acids, about 15 amino acids to about 55 amino acids, about 15 amino acids to about 50 amino acids, about 15 amino acids to about 45 amino acids, about 15 amino acids to about 40 amino acids, about 15 amino acids to about 35 amino acids, about 15 amino acids to about 30 amino acids, about 15 amino acids to about 25 amino acids, about 15 amino acids to about 20 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 20 amino acids to about 100 amino acids, about 20 amino acids to about 95 amino acids, about 20 amino acids to about 90 amino acids, about 20 amino acids to about 85 amino acids, about 20 amino acids to about 80 amino acids, about 20 amino acids to about 75 amino acids, about 20 amino acids to about 70 amino acids, about 20 amino acids to about 65 amino acids, about 20 amino acids to about 60 amino acids, about 20 amino acids to about 55 amino acids, about 20 amino acids to about 50 amino acids, about 20 amino acids to about 45 amino acids, about 20 amino acids to about 40 amino acids, about 20 amino acids to about 35 amino acids, about 20 amino acids to about 30 amino acids, about 20 amino acids to about 25 amino acids, about 25 amino acids to about 100 amino acids, about 25 amino acids to about 95 amino acids, about 25 amino acids to about 90 amino acids, about 25 amino acids to about 85 amino acids, about 25 amino acids to about 80 amino acids, about 25 amino acids to about 75 amino acids, about 25 amino acids to about 70 amino acids, about 25 amino acids to about 65 amino acids, about 25 amino acids to about 60 amino acids, about 25 amino acids to about 55 amino acids, about 25 amino acids to about 50 amino acids, about 25 amino acids to about 45 amino acids, about 25 amino acids to about 40 amino acids, about 25 amino acids to about 35 amino acids, about 25 amino acids to about 30 amino acids, about 30 amino acids to about 100 amino acids, about 30 amino acids to about 95 amino acids, about 30 amino acids to about 90 amino acids, about 30 amino acids to about 85 amino acids, about 30 amino acids to about 80 amino acids, about 30 amino acids to about 75 amino acids, about 30 amino acids to about 70 amino acids, about 30 amino acids to about 65 amino acids, about 30 amino acids to about 60 amino acids, about 30 amino acids to about 55 amino acids, about 30 amino acids to about 50 amino acids, about 30 amino acids to about 45 amino acids, about 30 amino acids to about 40 amino acids, about 30 amino acids to about 35 amino acids, about 35 amino acids to about 100 amino acids, about 35 amino acids to about 95 amino acids, about 35 amino acids to about 90 amino acids, about 35 amino acids to about 85 amino acids, about 35 amino acids to about 80 amino acids, about 35 amino acids to about 75 amino acids, about 35 amino acids to about 70 amino acids, about 35 amino acids to about 65 amino acids, about 35 amino acids to about 60 amino acids, about 35 amino acids to about 55 amino acids, about 35 amino acids to about 50 amino acids, about 35 amino acids to about 45 amino acids, about 35 amino acids to about 40 amino acids, about 40 amino acids to about 100 amino acids, about 40 amino acids to about 95 amino acids, about 40 amino acids to about 90 amino acids, about 40 amino acids to about 85 amino acids, about 40 amino acids to about 80 amino acids, about 40 amino acids to about 75 amino acids, about 40 amino acids to about 70 amino acids, about 40 amino acids to about 65 amino acids, about 40 amino acids to about 60 amino acids, about 40 amino acids to about 55 amino acids, about 40 amino acids to about 50 amino acids, about 40 amino acids to about 45 amino acids, about 45 amino acids to about 100 amino acids, about 45 amino acids to about 95 amino acids, about 45 amino acids to about 90 amino acids, about 45 amino acids to about 85 amino acids, about 45 amino acids to about 80 amino acids, about 45 amino acids to about 75 amino acids, about 45 amino acids to about 70 amino acids, about 45 amino acids to about 65 amino acids, about 45 amino acids to about 60 amino acids, about 45 amino acids to about 55 amino acids, about 45 amino acids to about 50 amino acids, about 50 amino acids to about 100 amino acids, about 50 amino acids to about 95 amino acids, about 50 amino acids to about 90 amino acids, about 50 amino acids to about 85 amino acids, about 50 amino acids to about 80 amino acids, about 50 amino acids to about 75 amino acids, about 50 amino acids to about 70 amino acids, about 50 amino acids to about 65 amino acids, about 50 amino acids to about 60 amino acids, about 50 amino acids to about 55 amino acids, about 55 amino acids to about 100 amino acids, about 55 amino acids to about 95 amino acids, about 55 amino acids to about 90 amino acids, about 55 amino acids to about 85 amino acids, about 55 amino acids to about 80 amino acids, about 55 amino acids to about 75 amino acids, about 55 amino acids to about 70 amino acids, about 55 amino acids to about 65 amino acids, about 55 amino acids to about 60 amino acids, about 60 amino acids to about 100 amino acids, about 60 amino acids to about 95 amino acids, about 60 amino acids to about 90 amino acids, about 60 amino acids to about 85 amino acids, about 60 amino acids to about 80 amino acids, about 60 amino acids to about 75 amino acids, about 60 amino acids to about 70 amino acids, about 60 amino acids to about 65 amino acids, about 65 amino acids to about 100 amino acids, about 65 amino acids to about 95 amino acids, about 65 amino acids to about 90 amino acids, about 65 amino acids to about 85 amino acids, about 65 amino acids to about 80 amino acids, about 65 amino acids to about 75 amino acids, about 65 amino acids to about 70 amino acids, about 70 amino acids to about 100 amino acids, about 70 amino acids to about 95 amino acids, about 70 amino acids to about 90 amino acids, about 70 amino acids to about 85 amino acids, about 70 amino acids to about 80 amino acids, about 70 amino acids to about 75 amino acids, about 75 amino acids to about 100 amino acids, about 75 amino acids to about 95 amino acids, about 75 amino acids to about 90 amino acids, about 75 amino acids to about 85 amino acids, about 75 amino acids to about 80 amino acids, about 80 amino acids to about 100 amino acids, about 80 amino acids to about 95 amino acids, about 80 amino acids to about 90 amino acids, about 80 amino acids to about 85 amino acids, about 85 amino acids to about 100 amino acids, about 85 amino acids to about 95 amino acids, about 85 amino acids to about 90 amino acids, about 90 amino acids to about 100 amino acids, about 90 amino acids to about 95 amino acids, or about 95 amino acids to about 100 amino acids.

A linker peptide sequence can be or include a non-naturally occurring sequence (e.g., a synthetic sequence). In some embodiments, a linker peptide sequence can include a sequence from a naturally occurring polypeptide (e.g., a domain). In some embodiments of any of the linker peptide sequences described herein, the linker peptide sequence is a flexible sequence (e.g., does not form an alpha alpha-helix or a beta-strand). In some embodiments of any of the linker peptide sequences described herein, the linker peptide sequence (or a portion thereof) forms an alpha-helix. In some embodiments of any of the linker peptides sequences described herein, the linker peptide sequence (or a portion thereof) forms a beta-strand.

In some embodiments, a linker peptide sequence can include one or more of the following amino acid sequences: GSGSGSGSGS (SEQ ID NO: 22), GSGSGSGS (SEQ ID NO: 23), or RSGSGSGS (SEQ ID NO: 24). In some embodiments, linker peptide sequence can be encoded by one or more of the following nucleic acid sequences:

(SEQ ID NO: 25)
GGATCCGGCAGCGGATCTGGCAGTGGAAGC,
(SEQ ID NO: 26)
GGATCTGGCTCTGGAAGCGGCAGC,
(SEQ ID NO: 27)
AGATCCGGATCTGGAAGTGGCTCC,
or
(SEQ ID NO: 28)
GGAAGTGGATCTGGGAGCGGCTCT.

In some embodiments, a linker peptide sequence can include one or more (e.g., two, three, or four) copies of any one of SEQ ID NOs: 22-24, e.g., in tandem.

In some embodiments, a linker peptide sequence can be or can include one or more of SEQ ID NO: 23, 24, or 31. In some embodiments, a linker peptide sequence can be or include GS (which can, e.g., be encoded by the DNA sequence of ggatcc).

(DNA sequence encoding the exemplary linker
peptide sequence of SEQ ID NO: 23)

SEQ ID NO: 29

ggatcaggcagtggctctggcagc
(DNA sequence encoding the exemplary linker
peptide sequence of SEQ ID NO: 24)

SEQ ID NO: 30

ggatctggaagtggctcc
(DNA sequence encoding the exemplary linker
peptide sequence of SEQ ID NO: 23)

SEQ ID NO: 28

ggaagtggatctgggagcggctct
((G4S)₃ linker peptide sequence)

SEQ ID NO: 31

GGGGSGGGGSGGGGS

Additional examples of linker peptide sequences that can be used in the present single-chain chimeric antigen receptors and multi-chain chimeric antigen receptors are known in the art.

### Antigens

In some embodiments, an antigen-binding domain described herein can bind to a single antigen (e.g., any of the exemplary antigens described herein or known in the art). In some embodiments, an antigen-binding domain described herein can bind to two or more different antigens (e.g., two or more of any of the exemplary antigens described herein or known in the art). Non-limiting examples of antigens include: HER2, A33 antigen, 9-0-acetyl-GD3, CA19-9 marker, BhCG, CA-125 marker, carboanhydrase IX (MN/CA IX), calreticulin, CCR5, CCR8, CD2, CD3, CD5, CD16, CD19, CD20, CD22, CD24, CD25, CD27, CD28, CD30, CD33, CD3δ, CD40L, CD44, CD44V6, CD63, CD70, CD84, CD96, CD100, CC123, CD133, CD138, CD150, CD152 (CTLA-4), CD160, CRTAM, CS1 (CD319), DNAM-1 (CD226), CD229, CD244, CD272 (BTLA), CD274 (PDL-1, B7H1), CD279 (PD-1), CD319, CD352, CRTAM (CD355), CD358, DR3, GITR (TNFRSF 18), HVEM, ICOS, LIGHT, LTBR, OX40, activating forms of KIR, NKG2C, NKG2D, NKG2E, one or more natural cytotoxicity receptors, NTB-A, PEN-5, carcinoma embryonic antigen (CEA; CD66e), desmoglein 4, E-cadherin neoepitope, endosialin, ephrin A2 (EphA2), epidermal growth factor receptor (EGFR), epithelial cell adhesion molecule (EpCAM), fucosyl GM1, GD2, GD3, GM2, ganglioside GM3, Globo H, glycoprotein 100, HER2/neu, HER3, HER4, insulin-like growth factor receptor 1, Lewis-Y, LG, Ly-6, melanoma-specific chondroitin-sulfate proteoglycan (MCSCP), mesothelin, MUC1, MUC2, MUC3, MUC4, MUC5AC, MUC5b, MUC7, MUC16, Mullerian inhibitory substance (MIS) receptor type II, plasma cell antigen, poly SA, PSCA, PSMA, sonic hedgehog (SHH), SAS, STEAP, sTn antigen, TNF-α precursor, 2B4 (CD244), β2-integrins, KIR, KIR2DL1, KIR2DL2, KIR2DL3, KIR3DL2, KIR-L, KLRGI, LAIR-1, NKG2A, NKR-P IA, Siglec-3, Siglec-7, Siglec-9, TCRa, TCRB, TCR5y, TIM1, LAG3, LAIR1, PD-1H, TIGIT, TIM2, and TIM3.

In some embodiments, the antigen-binding domain described herein can specifically bind to antigen selected from the group of: MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGI, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

Additional examples of antigens are known in the art.

### Intracellular Signaling Domains

An example of an intracellular signaling domain is the intracellular signaling domain of Lck (also variously known as lymphocyte-specific protein tyrosine kinase, LCK proto-oncogene, Src family tyrosine kinase, IMD22, LSK, YT16, p56lck, or pp58lck). Lck is a member of the Src family of tyrosine kinases that is found in lymphocytes. Lck phosphorylates tyrosine residues of various proteins involved in the intracellular signaling pathways of lymphocytes. In some embodiments, an intracellular signaling domain can be an intracellular signaling domain from human Lck polypeptide. An exemplary polypeptide sequence of a human Lck (e.g., UniProt Protein Accession: P06239, found at URL www.uniprot.org/uniprot/P06239) with an exemplary intracellular signaling domain underlined is SEQ ID NO: 32. An intracellular signaling domain can be a portion of the exemplary intracellular signaling domain underlined in SEQ ID NO: 32, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 32.

(Human Lck with an exemplary intracellular
signaling domain underlined)
SEQ ID NO: 32
MGCGCSSHPEDDWMENIDVCENCHYPIVPLDGKGTLLIRNGSEVRDPLVT
YEGSNPPASPLQDNLVIALHSYEPSHDGDLGFEKGEQLRILEQSGEWWKA
QSLTTGQEGFIPFNFVAKANSLEPEPWFFKNLSRKDAERQLLAPGNTHGS
FLIRESESTAGSFSLSVRDFDQNQGEVVKHYKIRNLDNGGFYISPRITFP
GLHELVRHYTNASDGLCTRLSRPCQTQKPQKPWWEDEWEVPRETLKLVER
LGAGQFGEVWMGYYNGHTKVAVKSLKQGSMSPDAFLAEANLMKQLQHQRL
VRLYAVVTQEPIYIITEYMENGSLVDFLKTPSGIKLTINKLLDMAAQIAE
GMAFIEERNYIHRDLRAANILVSDTLSCKIADFGLARLIEDNEYTAREGA
KFPIKWTAPEAINYGTFTIKSDVWSFGILLTEIVTHGRIPYPGMTNPEVI
QNLERGYRMVRPDNCPEELYQLMRLCWKERPEDRPTFDYLRSVLEDFFTA
TEGQYQPQP

In some embodiments, an intracellular signaling domain can be an intracellular signaling domain from mouse Lck polypeptide. An exemplary polypeptide sequence of a mouse Lck (e.g., a polypeptide as shown in UniProt Protein Accession: P06240, found at URL www.uniprot.org/uniprot/P06240) with an exemplary intracellular signaling domain underlined is SEQ ID NO: 33. An intracellular signaling domain can be a portion of the exemplary intracellular signaling domain underlined in SEQ ID NO: 33, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 33.

(Mouse Lck polypeptide with an exemplary
intracellular signaling domain underlined)
SEQ ID NO: 33
MGCVCSSNPEDDWMENIDVCENCHYPIVPLDSKISLPIRNGSEVRDPLVT
YEGSLPPASPLQDNLVIALHSYEPSHDGDLGFEKGEQLRILEQSGEWWKA
QSLTTGQEGFIPFNFVAKANSLEPEPWFFKNLSRKDAERQLLAPGNTHGS
FLIRESESTAGSFSLSVRDFDQNQGEVVKHYKIRNLDNGGFYISPRITFP
GLHDLVRHYTNASDGLCTKLSRPCQTQKPQKPWWEDEWEVPRETLKLVER
LGAGQFGEVWMGYYNGHTKVAVKSLKQGSMSPDAFLAEANLMKQLQHPRL
VRLYAVVTQEPIYIITEYMENGSLVDFLKTPSGIKLNVNKLLDMAAQIAE
GMAFIEEQNYIHRDLRAANILVSDTLSCKIADFGLARLIEDNEYTAREGA
KFPIKWTAPEAINYGTFTIKSDVWSFGILLTEIVTHGRIPYPGMTNPEVI
QNLERGYRMVRPDNCPEELYHLMMLCWKERPEDRPTFDYLRSVLDDFFTA
TEGQYQPQP

In some embodiments, an intracellular signaling domain can be an intracellular signaling domain from FasR polypeptide (also variously known as Fas receptor, ALPSIA, APO-1, APT1, CD95, FAS1, FASTM, TNFRSF6, or Fas cell surface death receptor). The Fas receptor is encoded by the FasR gene, and is a death receptor on the surface of cells that leads to apoptosis.

In some embodiments, an intracellular signaling domain can be an intracellular signaling domain from human FasR polypeptide. An exemplary polypeptide sequence of a human FasR (e.g., UniProt Protein Accession: P25445, found at URL www.uniprot.org/uniprot/P25445) with an exemplary cytoplasmic signaling domain underlined is SEQ ID NO: 34. An intracellular signaling domain can be a portion of the exemplary intracellular signaling domain underlined in SEQ ID NO: 34, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 34.

(Human FasR polypeptide with an exemplary
intracellular signaling domain underlined)
SEQ ID NO: 34
MLGIWTLLPLVLTSVARLSSKSVNAQVTDINSKGLELRKTVTTVETQNLE
GLHHDGQFCHKPCPPGERKARDCTVNGDEPDCVPCQEGKEYTDKAHFSSK
CRRCRLCDEGHGLEVEINCTRTQNTKCRCKPNFFCNSTVCEHCDPCTKCE
HGIIKECTLTSNTKCKEEGSRSNLGWLCLLLLPIPLIVWVKRKEVQKTCR
KHRKENQGSHESPTLNPETVAINLSDVDLSKYITTIAGVMTLSQVKGFVR
KNGVNEAKIDEIKNDNVQDTAEQKVQLLRNWHQLHGKKEAYDTLIKDLKK
ANLCTLAEKIQTIILKDITSDSENSNFRNEIQSLV

In some embodiments, an intracellular signaling domain can be an intracellular signaling domain of mouse FasR polypeptide. An exemplary polypeptide sequence of a mouse FasR (e.g., a polypeptide as shown in UniProt Protein Accession: P25446, found at URL www.uniprot.org/uniprot/P25446) with an exemplary cytoplasmic signaling domain underlined is SEQ ID NO: 35. An intracellular signaling domain can be a portion of the exemplary intracellular signaling domain underlined in SEQ ID NO: 35, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 35.

(Mouse FasR polypeptide with an exemplary
intracellular signaling domain underlined)
SEQ ID NO: 35
MLWIWAVLPLVLAGSQLRVHTQGTNSISESLKLRRRVRETDKNCSEGLYQ
GGPFCCQPCQPGKKKVEDCKMNGGTPTCAPCTEGKEYMDKNHYADKCRRC
TLCDEEHGLEVETNCTLTQNTKCKCKPDFYCDSPGCEHCVRCASCEHGTL
EPCTATSNTNCRKQSPRNRLWLLTILVLLIPLVFIYRKYRKRKCWKRRQD
DPESRTSSRETIPMNASNLSLSKYIPRIAEDMTIQEAKKFARENNIKEGK
IDEIMHDSIQDTAEQKVQLLLCWYQSHGKSDAYQDLIKGLKKAECRRTLD
KFQDMVQKDLGKSTPDTGNENEGQCLE

In some embodiments, an intracellular signaling domain that is a domain from TNFR-I (also variously known as tumor necrosis factor receptor 1, TNFRSFIA, CD120a, FPF, MS5, TBP1, TNF-R, TNF—R—I, TNF-R55, TNFAR, TNFR1, TNFR1-d2, TNFR55, TNFR60, p55, p55-R, p60, tumor necrosis factor receptor superfamily member 1A, or TNF receptor superfamily member 1A). TNFR-I is a membrane receptor that binds tumor necrosis factor-alpha (TNFα). In some embodiments, an intracellular signaling domain can be an intracellular signaling domain of human TNFR-I polypeptide. An exemplary polypeptide sequence of a human TNFR-I (e.g., UniProt Protein Accession: P19438, found at URL www.uniprot.org/uniprot/P19438) with an exemplary cytoplasmic signaling domain underlined is SEQ ID NO: 36. An intracellular signaling domain can be a portion of the exemplary intracellular signaling domain underlined in SEQ ID NO: 36, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 36.

(Human TNFR-I polypeptide with an exemplary
intracellular signaling domain underlined)
SEQ ID NO: 36
MGLSTVPDLLLPLVLLELLVGIYPSGVIGLVPHLGDREKRDSVCPQGKYI
HPQNNSICCTKCHKGTYLYNDCPGPGQDTDCRECESGSFTASENHLRHCL
SCSKCRKEMGQVEISSCTVDRDTVCGCRKNQYRHYWSENLFQCFNCSLCL
NGTVHLSCQEKQNTVCTCHAGFFLRENECVSCSNCKKSLECTKLCLPQIE
NVKGTEDSGTTVLLPLVIFFGLCLLSLLFIGLMYRYQRWKSKLYSIVCGK
STPEKEGELEGTTTKPLAPNPSFSPTPGFTPTLGFSPVPSSTFTSSSTYT
PGDCPNFAAPRREVAPPYQGADPILATALASDPIPNPLQKWEDSAHKPQS
LDTDDPATLYAVVENVPPLRWKEFVRRLGLSDHEIDRLELQNGRCLREAQ
YSMLATWRRRTPRREATLELLGRVLRDMDLLGCLEDIEEALCGPAALPPA
PSLLR

In some embodiments, an intracellular signaling domain can be an intracellular domain from a mouse TNFR-I polypeptide. An exemplary polypeptide sequence of a mouse TNFR-I (e.g., a polypeptide as shown in UniProt Protein Accession: P25118, found at URL www.uniprot.org/uniprot/P25118) with an exemplary cytoplasmic signaling domain underlined is SEQ ID NO: 37. An intracellular signaling domain can be a portion of the exemplary intracellular signaling domain underlined in SEQ ID NO: 37, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 37.

(Mouse TNFR-I polypeptide with an exemplary
intracellular signaling domain underlined)
SEQ ID NO: 37
MGLPTVPGLLLSLVLLALLMGIHPSGVTGLVPSLGDREKRDSLCPQGKYV
HSKNNSICCTKCHKGTYLVSDCPSPGRDTVCRECEKGTFTASQNYLRQCL
SCKTCRKEMSQVEISPCQADKDTVCGCKENQFQRYLSETHFQCVDCSPCF
NGTVTIPCKETQNTVCNCHAGFFLRESECVPCSHCKKNEECMKLCLPPPL
ANVTNPQDSGTAVLLPLVILLGLCLLSFIFISLMCRYPRWRPEVYSIICR
DPVPVKEEKAGKPLTPAPSPAFSPTSGFNPTLGFSTPGFSSPVSSTPISP
IFGPSNWHFMPPVSEVVPTQGADPLLYESLCSVPAPTSVQKWEDSAHPQR
PDNADLAILYAVVDGVPPARWKEFMRFMGLSEHEIERLEMQNGRCLREAQ
YSMLEAWRRRTPRHEDTLEVVGLVLSKMNLAGCLENILEALRNPAPSSTT
RLPR

In some embodiments, an intracellular signaling domain can be an intracellular signaling domain from TNFR-II (also variously known as tumor necrosis factor receptor 2, TNFRSF1B, CD120b, TBPII, TNF—R-II, TNF-R75, TNFBR, TNFRIB, TNFR2, TNFR80, p75, p75TNFR, tumor necrosis factor receptor superfamily member 1B, or TNF receptor superfamily member 1B). TNFR-II is a membrane receptor that binds tumor necrosis factor-alpha (TNFα). In some embodiments, an intracellular signaling domain can be an intracellular signaling domain from a human TNFR-II polypeptide. An exemplary polypeptide sequence of a human TNFR-II (e.g., UniProt Protein Accession: P20333, found at URL www.uniprot.org/uniprot/P20333) with an exemplary cytoplasmic signaling domain underlined is SEQ ID NO: 38. An intracellular signaling domain can be a portion of the exemplary intracellular signaling domain underlined in SEQ ID NO: 38, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 38.

(Human TNFR-II polypeptide with an exemplary
intracellular signaling domain underlined)
SEQ ID NO: 38
MAPVAVWAALAVGLELWAAAHALPAQVAFTPYAPEPGSTCRLREYYDQTA
QMCCSKCSPGQHAKVFCTKTSDTVCDSCEDSTYTQLWNWVPECLSCGSRC
SSDQVETQACTREQNRICTCRPGWYCALSKQEGCRLCAPLRKCRPGFGVA
RPGTETSDVVCKPCAPGTFSNTTSSTDICRPHQICNVVAIPGNASMDAVC
TSTSPTRSMAPGAVHLPQPVSTRSQHTQPTPEPSTAPSTSFLLPMGPSPP
AEGSTGDFALPVGLIVGVTALGLLIIGVVNCVIMTQVKKKPLCLQREAKV
PHLPADKARGTQGPEQQHLLITAPSSSSSSLESSASALDRRAPTRNQPQA
PGVEASGAGEARASTGSSDSSPGGHGTQVNVTCIVNVCSSSDHSSQCSSQ
ASSTMGDTDSSPSESPKDEQVPFSKEECAFRSQLETPETLLGSTEEKPLP
LGVPDAGMKPS

In some embodiments, an intracellular signaling domain is an intracellular signaling domain from a mouse TNFR-II polypeptide. An exemplary polypeptide sequence of a mouse TNFR-II (e.g., a polypeptide as shown in UniProt Protein Accession: P25119, found at URL www.uniprot.org/uniprot/P25119) with an exemplary cytoplasmic signaling domain underlined is SEQ ID NO: 39. An intracellular signaling domain can be a portion of the exemplary intracellular signaling domain underlined in SEQ ID NO: 39, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 39.

(Mouse TNFR-II polypeptide with an exemplary
intracellular signaling domain underlined)
SEQ ID NO: 39
MAPAALWVALVFELQLWATGHTVPAQVVLTPYKPEPGYECQISQEYYDRK
AQMCCAKCPPGQYVKHFCNKTSDTVCADCEASMYTQVWNQFRTCLSCSSS
CTTDQVEIRACTKQQNRVCACEAGRYCALKTHSGSCRQCMRLSKCGPGFG
VASSRAPNGNVLCKACAPGTFSDTTSSTDVCRPHRICSILAIPGNASTDA
VCAPESPTLSAIPRTLYVSQPEPTRSQPLDQEPGPSQTPSILTSLGSTPI
IEQSTKGGISLPIGLIVGVTSLGLLMLGLVNCIILVQRKKKPSCLQRDAK
VPHVPDEKSQDAVGLEQQHLLTTAPSSSSSSLESSASAGDRRAPPGGHPQ
ARVMAEAQGFQEARASSRISDSSHGSHGTHVNVTCIVNVCSSSDHSSQCS
SQASATVGDPDAKPSASPKDEQVPFSQEECPSQSPCETTETLQSHEKPLP
LGVPDMGMKPSQAGWFDQIAVKVA

In some embodiments, an intracellular signaling domain is an intracellular signaling domain is from LIGHT (also variously known as tumor necrosis factor superfamily member 14, TNFSF14, CD258, HVEML, LIGHT, LTg, TR2, TNLGID, tumor necrosis factor superfamily member 14, or TNF superfamily member 14). LIGHT is a secreted protein of the TNF superfamily, has been shown to stimulate the proliferation of T cells, and trigger apoptosis of various tumor cells, and has been shown to interact with TNFRSF14, TNFRSF6B, BIRC2, TRAF2, and TRAF3. In some embodiments, an intracellular signaling domain is an intracellular signaling domain from a human LIGHT polypeptide. An exemplary polypeptide sequence of a human LIGHT (e.g., UniProt Protein Accession: 043557, found at URL www.uniprot.org/uniprot/O43557) with an exemplary cytoplasmic signaling domain underlined is SEQ ID NO: 40. An intracellular signaling domain can be a portion of the exemplary intracellular signaling domain underlined in SEQ ID NO: 40, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 40.

(Human LIGHT polypeptide with an exemplary
intracellular signaling domain underlined)
SEQ ID NO: 40
MEESVVRPSVFVVDGQTDIPFTRLGRSHRRQSCSVARVGLGLLLLLMGAG
LAVQGWFLLQLHWRLGEMVTRLPDGPAGSWEQLIQERRSHEVNPAAHLTG
ANSSLTGSGGPLLWETQLGLAFLRGLSYHDGALVVTKAGYYYIYSKVQLG
GVGCPLGLASTITHGLYKRTPRYPEELELLVSQQSPCGRATSSSRVWWDS
SFLGGVVHLEAGEKVVVRVLDERLVRLRDGTRSYFGAFMV

In some embodiments, an intracellular signaling domain is an intracellular signaling domain from a mouse LIGHT polypeptide. An exemplary polypeptide sequence of a mouse LIGHT (e.g., a polypeptide as shown in UniProt Protein Accession: Q9QYH9, found at URL www.uniprot.org/uniprot/Q9QYH9) with an exemplary cytoplasmic signaling domain underlined is SEQ ID NO: 41. An intracellular signaling domain can be a portion of the exemplary intracellular signaling domain underlined in SEQ ID NO: 41, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 41.

(Mouse LIGHT polypeptide with an exemplary
intracellular signaling domain underlined)
SEQ ID NO: 41
MESVVQPSVFVVDGQTDIPFRRLEQNHRRRRCGTVQVSLALVLLLGAGLA
TQGWFLLRLHQRLGDIVAHLPDGGKGSWEKLIQDQRSHQANPAAHLTGAN
ASLIGIGGPLLWETRLGLAFLRGLTYHDGALVTMEPGYYYVYSKVQLSGV
GCPQGLANGLPITHGLYKRTSRYPKELELLVSRRSPCGRANSSRVWWDSS
FLGGVVHLEAGEEVVVRVPGNRLVRPRDGTRSYFGAFMV

In some embodiments, an intracellular signaling domain can be an intracellular signaling domain from ICOS (also variously known as CD278, AILIM, CVID1, inducible T-cell co-stimulator, or inducible T-cell costimulator). ICOS is an immune checkpoint protein that is a member of the CD28-superfamily costimulatory molecule and is expressed on activated T cells. In some embodiments, an intracellular signaling domain is an intracellular signaling domain from a human ICOS polypeptide. An exemplary polypeptide sequence of a human ICOS (e.g., UniProt Protein Accession: Q9Y6W8, found at URL www.uniprot.org/uniprot/Q9Y6W8) with an exemplary intracellular signaling domain underlined is SEQ ID NO: 42. An intracellular signaling domain can be a portion of the exemplary intracellular signaling domain underlined in SEQ ID NO: 42, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 42.

(Human ICOS polypeptide with an exemplary
intracellular signaling domain underlined)
SEQ ID NO: 42
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKKYSSSVHDPNGEYMFMRAVNTAKKSRLTDVTL

In some embodiments, an intracellular signaling domain is an intracellular signaling domain from a mouse ICOS polypeptide. An exemplary polypeptide sequence of a mouse ICOS (e.g., a polypeptide as shown in UniProt Protein Accession: Q9WVS0, found at URL www.uniprot.org/uniprot/Q9WVS0) with an exemplary intracellular signaling domain underlined is SEQ ID NO: 43. An intracellular signaling domain can be a portion of the exemplary intracellular signaling domain underlined in SEQ ID NO: 43, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 43.

(Mouse ICOS polypeptide with an exemplary
intracellular signaling domain underlined)
SEQ ID NO: 43
MKPYFCRVFVFCFLIRLLTGEINGSADHRMFSFHNGGVQISCKYPETVQQ
LKMRLFREREVLCELTKTKGSGNAVSIKNPMLCLYHLSNNSVSFFLNNPD
SSQGSYYFCSLSIFDPPPFQERNLSGGYLHIYESQLCCQLKLWLPVGCAA
FVVVLLFGCILIIWFSKKKYGSSVHDPNSEYMFMAAVNTNKKSRLAGVTS

In some embodiments, an intracellular signaling domain can be an intracellular signaling domain from 4-1BB (also variously known as TNFRSF9, CD137, CDw137, ILA, tumor necrosis factor receptor superfamily member 9, or TNF receptor superfamily member 9). 4-1BB is a member of the tumor necrosis factor (TNF) receptor family that contributes to costimulatory activity for activated T cells. In some embodiments, an intracellular signaling domain is an intracellular signaling domain from a human 4-1BB polypeptide. An exemplary polypeptide sequence of a human 4-1BB (e.g., UniProt Protein Accession: Q07011, found at URL www.uniprot.org/uniprot/Q07011) with an exemplary intracellular signaling domain underlined is SEQ ID NO: 44. An intracellular signaling domain can be a portion of the exemplary intracellular signaling domain underlined in SEQ ID NO: 44, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 44.

(Human 4-1BB polypeptide with an exemplary
intracellular signaling domain underlined)
SEQ ID NO: 44
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In some embodiments, an intracellular signaling domain can be or can include a sequence that is at least 50% (at least 55%, at least 60%, at least 65%, at least 70%, at least 75%, at least 80%, at least 85%, at least 90%, at least 95%, at least 99%, or 100%) identical to SEQ ID NO: 45.

(4-1BB intracellular signaling domain)
SEQ ID NO: 45
KRGRKKLLYIFKQPFMRPVQTTQEEDGCSCRFPEEEEGGCEL
(DNA sequence encoding the 4-1BB intracellular
signaling domain of SEQ ID NO: 45)
SEQ ID NO: 46
AAAAGGGGCCGGAAAAAGCTGCTGTATATTTTCAAACAGCCTTTTATGAG
GCCTGTGCAGACAACACAGGAAGAGGACGGCTGTAGCTGTCGGTTCCCCG
AAGAGGAAGAGGGGGGCTGCGAACTG

In some embodiments, an intracellular signaling domain can be an intracellular domain from amino acids 214 to 255 of SEQ ID NO: 44 (human 4-1BB). In some embodiments, a costimulatory domain is or includes a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to amino acids 214 to 255 of SEQ ID NO: 44, or a portion thereof.

(Human 4-1BB polypeptide)
SEQ ID NO: 44
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In some embodiments, an intracellular signaling domain can be or can include a sequence that is at least 50% (at least 55%, at least 60%, at least 65%, at least 70%, at least 75%, at least 80%, at least 85%, at least 90%, at least 95%, at least 99%, or 100%) identical to SEQ ID NO: 47.

(Human 4-1BB intracellular signaling domain)
SEQ ID NO: 47
KRGRKKLLYIFKQPFMRPVQTTQEEDGCSCRFPEEEEGGCE
(DNA encoding the 4-1BB intracellular signaling
domain of SEQ ID NO: 47)
SEQ ID NO: 48
aaacggggcagaaagaaactcctgtatatattcaaacaaccatttatgag
accagtacaaactactcaagaggaagatggctgtagctgccgatttccag
aagaagaagaaggaggatgtgaa

In some embodiments, an intracellular signaling domain can be an intracellular signaling domain from a mouse 4-1BB polypeptide. An exemplary polypeptide sequence of a mouse 4-1BB (e.g., a polypeptide as shown in UniProt Protein Accession: P20334, found at URL www.uniprot.org/uniprot/P20334) with an exemplary intracellular signaling domain underlined is SEQ ID NO: 49. An intracellular signaling domain can be a portion of the exemplary intracellular signaling domain underlined in SEQ ID NO: 49, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 49.

(Mouse 4-1BB polypeptide with an exemplary
intracellular signaling domain underlined)
SEQ ID NO: 49
MGNNCYNVVVIVLLLVGCEKVGAVQNSCDNCQPGTFCRKYNPVCKSCPPS
TFSSIGGQPNCNICRVCAGYFRFKKFCSSTHNAECECIEGFHCLGPQCTR
CEKDCRPGQELTKQGCKTCSLGTFNDQNGTGVCRPWTNCSLDGRSVLKTG
TTEKDVVCGPPVVSFSPSTTISVTPEGGPGGHSLQVLTLFLALTSALLLA
LIFITLLFSVLKWIRKKFPHIFKQPFKKTTGAAQEEDACSCRCPQEEEGG
GGGYEL

In some embodiments, an intracellular signaling domain can be a domain from CD27 (also variously known as S152, S152. LPFS2, T14, TNFRSF7, Tp55, or CD27 molecule). CD27 is a member of the tumor necrosis factor (TNF) receptor superfamily and plays a role in the generation and long-term maintenance of T cell immunity. In some embodiments, an intracellular signaling domain can be an intracellular signaling domain from a human CD27 polypeptide. An exemplary polypeptide sequence of a human CD27 (e.g., UniProt Protein Accession: P26842, found at URL www.uniprot.org/uniprot/P26842) with an intracellular signaling domain underlined is SEQ ID NO: 50. An intracellular signaling domain can be a portion of the exemplary intracellular signaling domain underlined in SEQ ID NO: 50, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 50.

(Human CD27 polypeptide with an exemplary
intracellular signaling domain underlined)
SEQ ID NO: 50
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRILVIFSGMF
LVFTLAGALFLHQRRKYRSNKGESPVEPAEPCHYSCPREEEGSTIPIQED
YRKPEPACSP

In some embodiments, an intracellular signaling domain can be or can include a sequence that is at least 50% (at least 55%, at least 60%, at least 65%, at least 70%, at least 75%, at least 80%, at least 85%, at least 90%, at least 95%, at least 99%, or 100%) identical to SEQ ID NO: 51.

(CD27 Intracellular Signaling Domain)
SEQ ID NO: 51
QRRKYRSNKGESPVEPAEPCHYSCPREEEGSTIPIQEDYRKPEPACSP

In some embodiments, an intracellular signaling domain can be an intracellular signaling domain from a mouse CD27 polypeptide. An exemplary polypeptide sequence of a mouse CD27 (e.g., a polypeptide as shown in UniProt Protein Accession: P41272, found at URL www.uniprot.org/uniprot/P41272) with an exemplary intracellular signaling domain underlined is SEQ ID NO: 52. An intracellular signaling domain can be a portion of the exemplary intracellular signaling domain underlined in SEQ ID NO: 52, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 52.

(Mouse CD27 polypeptide with an exemplary
intracellular signaling domain underlined)
SEQ ID NO: 52
MAWPPPYWLCMLGTLVGLSATLAPNSCPDKHYWTGGGLCCRMCEPGTFFV
KDCEQDRTAAQCDPCIPGTSFSPDYHTRPHCESCRHCNSGFLIRNCTVTA
NAECSCSKNWQCRDQECTECDPPLNPALTRQPSETPSPQPPPTHLPHGTE
KPSWPLHRQLPNSTVYSQRSSHRPLCSSDCIRIFVTFSSMFLIFVLGAIL
FFHQRRNHGPNEDRQAVPEEPCPYSCPREEEGSAIPIQEDYRKPEPAFYP

In some embodiments, an intracellular signaling domain can be a domain from OX40 (also variously known as CD134, TNFRSF4, ACT35, IMD16, OX40, TXGPIL, tumor necrosis factor receptor superfamily member 4, or TNF receptor superfamily member 4). OX40 is a member of the tumor necrosis factor (TNF) receptor superfamily and is a secondary co-stimulatory immune checkpoint molecule. OX40 binds to its ligand OX40L. In some embodiments, an intracellular signaling domain can be an intracellular signaling domain from a human OX40 polypeptide. An exemplary polypeptide sequence of a human OX40 (e.g., UniProt Protein Accession: P43489, found at URL www.uniprot.org/uniprot/P43489) with an exemplary intracellular signaling domain underlined is SEQ ID NO: 53. An intracellular signaling domain can be a portion of the exemplary intracellular signaling domain underlined in SEQ ID NO: 53, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 53.

(Human OX40 polypeptide with an exemplary
intracellular signaling domain underlined)
SEQ ID NO: 53
MCVGARRLGRGPCAALLLLGLGLSTVTGLHCVGDTYPSNDRCCHECRPGN
GMVSRCSRSQNTVCRPCGPGFYNDVVSSKPCKPCTWCNLRSGSERKQLCT
ATQDTVCRCRAGTQPLDSYKPGVDCAPCPPGHFSPGDNQACKPWTNCTLA
GKHTLQPASNSSDAICEDRDPPATQPQETQGPPARPITVQPTEAWPRTSQ
GPSTRPVEVPGGRAVAAILGLGLVLGLLGPLAILLALYLLRRDQRLPPDA
HKPPGGGSFRTPIQEEQADAHSTLAKI

In some embodiments, an intracellular signaling domain can be or can include a sequence that is at least 50% (at least 55%, at least 60%, at least 65%, at least 70%, at least 75%, at least 80%, at least 85%, at least 90%, at least 95%, at least 99%, or 100%) identical to SEQ ID NO: 54.

(OX40 intracellular signaling domain)

SEQ ID NO: 54

ALYLLRRDQRLPPDAHKPPGGGSFRTPIQEEQADAHSTLAKI

In some embodiments, an intracellular signaling domain can be an intracellular signaling domain from a mouse OX40 polypeptide. An exemplary polypeptide sequence of a mouse OX40 (e.g., a polypeptide as shown in UniProt Protein Accession: P47741, found at URL www.uniprot.org/uniprot/P47741) with an exemplary intracellular signaling domain underlined is SEQ ID NO: 55. An intracellular signaling domain can be a portion of the exemplary intracellular signaling domain underlined in SEQ ID NO: 55, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 55.

(Mouse OX40 polypeptide with an exemplary
intracellular signaling domain underlined)
SEQ ID NO: 55
MYVWVQQPTALLLLALTLGVTARRLNCVKHTYPSGHKCCRECQPGHGMVS
RCDHTRDTLCHPCETGFYNEAVNYDTCKQCTQCNHRSGSELKQNCTPTQD
TVCRCRPGTQPRQDSGYKLGVDCVPCPPGHFSPGNNQACKPWTNCTLSGK
QTRHPASDSLDAVCEDRSLLATLLWETQRPTFRPTTVQSTTVWPRTSELP
SPPTLVTPEGPAFAVLLGLGLGLLAPLTVLLALYLLRKAWRLPNTPKPCW
GNSFRTPIQEEHTDAHFTLAKI

In some embodiments, an intracellular signaling domain can be an intracellular signaling domain from CD2 (also variously known as cluster of differentiation 2, LFA-2, SRBC, T11, or CD2 molecule). CD2 is a cell adhesion molecule found on the surface of T cells and natural killer (NK) cells, and acts as a co-stimulatory molecule on these cells. In some embodiments, an intracellular signaling domain can be an intracellular signaling domain from a human CD2 polypeptide. An exemplary polypeptide sequence of a human CD2 (e.g., UniProt Protein Accession: P06729, found at URL www.uniprot.org/uniprot/P06729) with an exemplary intracellular signaling domain underlined is SEQ ID NO: 56. An intracellular signaling domain can be a portion of the exemplary intracellular signaling domain underlined in SEQ ID NO: 56, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 56.

(Human CD2 polypeptide with an exemplary
intracellular signaling domain underlined)
SEQ ID NO: 56
MSFPCKFVASFLLIFNVSSKGAVSKEITNALETWGALGQDINLDIPSFQM
SDDIDDIKWEKTSDKKKIAQFRKEKETFKEKDTYKLFKNGTLKIKHLKTD
DQDIYKVSIYDTKGKNVLEKIFDLKIQERVSKPKISWTCINTTLTCEVMN
GTDPELNLYQDGKHLKLSQRVITHKWTTSLSAKFKCTAGNKVSKESSVEP
VSCPEKGLDIYLIIGICGGGSLLMVFVALLVFYITKRKKQRSRRNDEELE
TRAHRVATEERGRKPHQIPASTPQNPATSQHPPPPPGHRSQAPSHRPPPP
GHRVQHQPQKRPPAPSGTQVHQQKGPPLPRPRVQPKPPHGAAENSLSPSS
N

In some embodiments, an intracellular signaling domain can be an intracellular signaling domain from a mouse CD2 polypeptide. An exemplary polypeptide sequence of a mouse CD2 (e.g., a polypeptide as shown in UniProt Protein Accession: P08920, found at URL www.uniprot.org/uniprot/P08920) with an exemplary intracellular signaling domain underlined is SEQ ID NO: 57. An intracellular signaling domain can be a portion of the exemplary intracellular signaling domain underlined in SEQ ID NO: 57, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 57.

(Mouse CD2 polypeptide with an exemplary
intracellular signaling domain underlined)
SEQ ID NO: 57
MKCKFLGSFFLLFSLSGKGADCRDNETIWGVLGHGITLNIPNFQMTDDID
EVRWVRRGTLVAEFKRKKPPFLISETYEVLANGSLKIKKPMMRNDSGTYN
VMVYGTNGMTRLEKDLDVRILERVSKPMIHWECPNTTLTCAVLQGTDFEL
KLYQGETLLNSLPQKNMSYQWTNLNAPFKCEAINPVSKESKMEVVNCPEK
GLSFYVTVGVGAGGLLLVLLVALFIFCICKRRKRNRRRKDEELEIKASRT
STVERGPKPHSTPAAAAQNSVALQAPPPPGHHLQTPGHRPLPPGHRTREH
QQKKRPPPSGTQIHQQKGPPLPRPRVQPKPPCGSGDGVSLPPPN

In some embodiments, an intracellular signaling domain can be an intracellular signaling domain from CD5 (also variously known as cluster of differentiation 5, LEU1, T1, or CD5 molecule). CD5 is expressed on the surface of T cells and is upregulated on T cells upon strong activation. In some embodiments, an intracellular signaling domain can be an intracellular signaling domain from a human CD5 polypeptide. An exemplary polypeptide sequence of a human CD5 (e.g., UniProt Protein Accession: P06127, found at URL www.uniprot.org/uniprot/P06127) with an exemplary intracellular signaling domain underlined is SEQ ID NO: 58. An intracellular signaling domain can be a portion of the exemplary intracellular signaling domain underlined in SEQ ID NO: 58, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 58.

(Human CD5 polypeptide with an exemplary
intracellular signaling domain underlined)
SEQ ID NO: 58
MPMGSLQPLATLYLLGMLVASCLGRLSWYDPDFQARLTRSNSKCQGQLEV
YLKDGWHMVCSQSWGRSSKQWEDPSQASKVCQRLNCGVPLSLGPFLVTYT
PQSSIICYGQLGSFSNCSHSRNDMCHSLGLTCLEPQKTTPPTTRPPPTTT
PEPTAPPRLQLVAQSGGQHCAGVVEFYSGSLGGTISYEAQDKTQDLENFL
CNNLQCGSFLKHLPETEAGRAQDPGEPREHQPLPIQWKIQNSSCTSLEHC
FRKIKPQKSGRVLALLCSGFQPKVQSRLVGGSSICEGTVEVRQGAQWAAL
CDSSSARSSLRWEEVCREQQCGSVNSYRVLDAGDPTSRGLFCPHQKLSQC
HELWERNSYCKKVFVTCQDPNPAGLAAGTVASIILALVLLVVLLVVCGPL
AYKKLVKKFRQKKQRQWIGPTGMNQNMSFHRNHTATVRSHAENPTASHVD
NEYSQPPRNSHLSAYPALEGALHRSSMQPDNSSDSDYDLHGAQRL

In some embodiments, an intracellular signaling domain can be an intracellular signaling domain from a mouse CD5 polypeptide. An exemplary polypeptide sequence of a mouse CD5 (e.g., a polypeptide as shown in UniProt Protein Accession: P13379, found at URL www.uniprot.org/uniprot/P13379) with an intracellular signaling domain underlined is SEQ ID NO: 59. An intracellular signaling domain can be a portion of the exemplary intracellular signaling domain underlined in SEQ ID NO: 59, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 59.

(Mouse CD5 polypeptide with an exemplary
intracellular signaling domain underlined)
SEQ ID NO: 59
MDSHEVLLAATYLLGTLAAFCLGQSGRGGLDIQVMLSGSNSKCQGQVEIQ
MENKWKTVCSSSWRLSQDHSKNAQQASAVCKQLRCGDPLALGPFPSLNRP
QNQVFCQGSPWSISNCNNTSSQDQCLPLSLICLEPQRTTPPPTTTPPTTV
PEPTAPPRLQLVPGHEGLRCTGVVEFYNGSWGGTILYKAKDRPLGLGNLI
CKSLQCGSFLTHLSGTEAAGTPAPAELRDPRPLPIRWEAPNGSCVSLQQC
FQKTTAQEGGQALTVICSDFQPKVQSRLVGGSSVCEGIAEVRQRSQWEAL
CDSSAARGRGRWEELCREQQCGDLISFHTVDADKTSPGFLCAQEKLSQCY
HLQKKKHCNKRVFVTCQDPNPAGLAPGTVASIILTLVLLVVLLAMCGPLV
YKKLVKKFRQKKQRQWIGPTGVNQNMSFHRSHTATVRSQVENPTASHVDN
EYSQPPRNSHLSAYPALEGALHRSSTQPDNSSDSDYDLQVAQRL

In some embodiments, an intracellular signaling domain can be an intracellular signaling domain from CD30 (also variously known as TNFRSF8, D1S166E, Ki-1, tumor necrosis factor receptor superfamily member 8, or TNF receptor superfamily member 8). CD30 is expressed by activated T and B cells and is involved with the activation of NF-kappaB. In some embodiments, an intracellular signaling domain can be an intracellular signaling domain from a human CD30 polypeptide. An exemplary polypeptide sequence of a human CD30 (e.g., UniProt Protein Accession: P28908, found at URL www.uniprot.org/uniprot/P28908) with an exemplary intracellular signaling domain underlined is SEQ ID NO: 60. An intracellular signaling domain can be a portion of the exemplary intracellular signaling domain underlined in SEQ ID NO: 60, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 60.

(Human CD30 polypeptide with an exemplary
intracellular signaling domain underlined)
SEQ ID NO: 60
MRVLLAALGLLFLGALRAFPQDRPFEDTCHGNPSHYYDKAVRRCCYRCPM
GLFPTQQCPQRPTDCRKQCEPDYYLDEADRCTACVTCSRDDLVEKTPCAW
NSSRVCECRPGMFCSTSAVNSCARCFFHSVCPAGMIVKFPGTAQKNTVCE
PASPGVSPACASPENCKEPSSGTIPQAKPTPVSPATSSASTMPVRGGTRL
AQEAASKLTRAPDSPSSVGRPSSDPGLSPTQPCPEGSGDCRKQCEPDYYL
DEAGRCTACVSCSRDDLVEKTPCAWNSSRTCECRPGMICATSATNSCARC
VPYPICAAETVTKPQDMAEKDTTFEAPPLGTQPDCNPTPENGEAPASTSP
TQSLLVDSQASKTLPIPTSAPVALSSTGKPVLDAGPVLFWVILVLVVVVG
SSAFLLCHRRACRKRIRQKLHLCYPVQTSQPKLELVDSRPRRSSTQLRSG
ASVTEPVAEERGLMSQPLMETCHSVGAAYLESLPLQDASPAGGPSSPRDL
PEPRVSTEHTNNKIEKIYIMKADTVIVGTVKAELPEGRGLAGPAEPELEE
ELEADHTPHYPEQETEPPLGSCSDVMLSVEEEGKEDPLPTAASGK

In some embodiments, an intracellular signaling domain can be an intracellular signaling domain from a mouse CD30 polypeptide. An exemplary polypeptide sequence of a mouse CD30 (e.g., a polypeptide as shown in UniProt Protein Accession: Q60846, found at URL www.uniprot.org/uniprot/Q60846) with an exemplary intracellular signaling domain underlined is SEQ ID NO: 61. An intracellular signaling domain can be a portion of the exemplary intracellular signaling domain underlined in SEQ ID NO: 61, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 61.

(Mouse CD30 polypeptide with an exemplary
intracellular signaling domain underlined)
SEQ ID NO: 61
MSALLTAAGLLFLGMLQAFPTDRPLKTTCAGDLSHYPGEAARNCCYQCPS
GLSPTQPCPRGPAHCRKQCAPDYYVNEDGKCTACVTCLPGLVEKAPCSGN
SPRICECQPGMHCCTPAVNSCARCKLHCSGEEVVKSPGTAKKDTICELPS
SGSGPNCSNPGDRKTLTSHATPQAMPTLESPANDSARSLLPMRVTNLVQE
DATELVKVPESSSSKAREPSPDPGNAEKNMTLELPSPGTLPDISTSENSK
EPASTASTLSLVVDAWTSSRMQPTSPLSTGTPFLDPGPVLFWVAMVVLLV
GSGSFLLCYWKACRRRFQQKFHLDYLVQTFQPKMEQTDSCPTEKLTQPQR
SGSVTDPSTGHKLSPVSPPPAVETCASVGATYLENLPLLDDSPAGNPFSP
REPPEPRVSTEHTNNRIEKIYIMKADTVIVGSVKTEVPEGRAPAGSTESE
LEAELEVDHAPHYPEQETEPPLGSCTEVMFSVEEGGKEDHGPTTVSEK

In some embodiments, an intracellular signaling domain can be an intracellular signaling domain from CD40 (also variously known as Bp50, CDW40, TNFRSF5, p50, or CD40 molecule). CD40 is a costimulatory protein found on antigen presenting cells and plays a role in their activation. In some embodiments, an intracellular signaling domain can be an intracellular signaling domain from a human CD40 polypeptide. An exemplary polypeptide sequence of a human CD40 (e.g., UniProt Protein Accession: P25942, found at URL www.uniprot.org/uniprot/P25942) with an exemplary intracellular signaling domain underlined is SEQ ID NO: 62. An intracellular signaling domain can be a portion of the exemplary intracellular signaling domain underlined in SEQ ID NO: 62, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 62.

(Human CD40 polypeptide with an exemplary
intracellular signaling domain underlined)
SEQ ID NO: 62
MVRLPLQCVLWGCLLTAVHPEPPTACREKQYLINSQCCSLCQPGQKLVSD
CTEFTETECLPCGESEFLDTWNRETHCHQHKYCDPNLGLRVQQKGTSETD
TICTCEEGWHCTSEACESCVLHRSCSPGFGVKQIATGVSDTICEPCPVGF
FSNVSSAFEKCHPWTSCETKDLVVQQAGTNKTDVVCGPQDRLRALVVIPI
IFGILFAILLVLVFIKKVAKKPTNKAPHPKQEPQEINFPDDLPGSNTAAP
VQETLHGCQPVTQEDGKESRISVQERQ

In some embodiments, an intracellular signaling domain can be or can include a sequence that is at least 50% (at least 55%, at least 60%, at least 65%, at least 70%, at least 75%, at least 80%, at least 85%, at least 90%, at least 95%, at least 99%, or 100%) identical to SEQ ID NO: 63.

(CD40 Intracellular Signaling Sequence)
SEQ ID NO: 63
KKVAKKPTNKAPHPKQEPQEINFPDDLPGSNTAAPVQETLHGCQPVTQ
(A)EDGKESRISVQERQ

In some embodiments, an intracellular signaling domain can be an intracellular signaling domain from a mouse CD40 polypeptide. An exemplary polypeptide sequence of a mouse CD40 (e.g., a polypeptide as shown in UniProt Protein Accession: P27512, found at URL www.uniprot.org/uniprot/P27512) with an exemplary intracellular signaling domain underlined is SEQ ID NO: 64. An intracellular signaling domain can be a portion of the exemplary intracellular signaling domain underlined in SEQ ID NO: 64, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 64.

(Mouse CD40 polypeptide with an exemplary
intracellular signaling domain underlined)
SEQ ID NO: 64
MVSLPRLCALWGCLLTAVHLGQCVTCSDKQYLHDGQCCDLCQPGSRLTSH
CTALEKTQCHPCDSGEFSAQWNREIRCHQHRHCEPNQGLRVKKEGTAESD
TVCTCKEGQHCTSKDCEACAQHTPCIPGFGVMEMATETTDTVCHPCPVGF
FSNQSSLFEKCYPWTSCEDKNLEVLQKGTSQTNVICGLKSRMRALLVIPV
VMGILITIFGVFLYIKKVVKKPKDNEILPPAARRQDPQEMEDYPGHNTAA
PVQETLHGCQPVTQEDGKESRISVQERQVTDSIALRPLV

In some embodiments, an intracellular signaling domain can be an intracellular signaling domain from CD28 (also variously known as cluster of differentiation 28, Tp44, or CD28 molecule). CD28 is a costimulatory protein expressed on T cells and provides co-stimulatory signals involved in T cell activation and survival. CD28 is the receptor for CD80 and CD86. In some embodiments, an intracellular signaling domain can be an intracellular signaling domain from a human CD28 polypeptide. An exemplary polypeptide sequence of a human CD28 (e.g., UniProt Protein Accession: P10747, found at URL www.uniprot.org/uniprot/P10747) with an exemplary intracellular signaling domain underlined is SEQ ID NO: 5. An intracellular signaling domain can be a portion of the exemplary intracellular signaling domain underlined in SEQ ID NO: 5, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 5.

(Human CD28 polypeptide with an exemplary
intracellular signaling domain underlined)
SEQ ID NO: 5
MLRLLLALNLFPSIQVTGNKILVKQSPMLVAYDNAVNLSCKYSYNLFSRE
FRASLHKGLDSAVEVCVVYGNYSQQLQVYSKTGFNCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In some embodiments, an intracellular signaling domain can be or can include a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical (or is identical) to amino acids 180 to 220 of SEQ ID NO: 5, or a fragment thereof.

In some embodiments, an intracellular signaling domain can be or can include a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical (or is identical) to amino acids 180 to 220 of SEQ ID NO: 65, or a fragment thereof.

(CD28 Intracellular Signaling Domain)

SEQ ID NO: 65

RSKRSRLLHSDYMNMTPRRPGPTRKHYQPYAPPRDFAAYRS

In some embodiments, an intracellular signaling domain can be an intracellular signaling domain from a mouse CD28 polypeptide. An exemplary polypeptide sequence of a mouse CD28 (e.g., a polypeptide as shown in UniProt Protein Accession: P31041, found at URL www.uniprot.org/uniprot/P31041) with an exemplary intracellular signaling domain underlined is SEQ ID NO: 66. An intracellular signaling domain can be a portion of the exemplary intracellular signaling domain underlined in SEQ ID NO: 66, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 66.

(Mouse CD28 polypeptide with an exemplary
intracellular signaling domain underlined)
SEQ ID NO: 66
MTLRLLFLALNFFSVQVTENKILVKQSPLLVVDSNEVSLSCRYSYNLLAK
EFRASLYKGVNSDVEVCVGNGNFTYQPQFRSNAEFNCDGDFDNETVTFRL
WNLHVNHTDIYFCKIEFMYPPPYLDNERSNGTIIHIKEKHLCHTQSSPKL
FWALVVVAGVLFCYGLLVTVALCVIWTNSRRNRLLQSDYMNMTPRRPGLT
RKPYQPYAPARDFAAYRP

In some embodiments, an intracellular signaling domain can be an intracellular signaling domain that from CD11a. CD11a is found on T-cells, B-cells, macrophages, neutrophils, and NK cells, and binds to CD11a on antigen-presenting cells. In some embodiments, an intracellular signaling domain can be an intracellular signaling domain from human CD11a polypeptide. In some embodiments, an intracellular signaling domain can be an intracellular signaling domain from a human CD11a polypeptide.

An exemplary polypeptide sequence of a human CD11a with an exemplary intracellular signaling domain underlined is SEQ ID NO: 67. An intracellular signaling domain can be a portion of the exemplary intracellular signaling domain underlined in SEQ ID NO: 67, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 67.

(Human CD11a with exemplary intracellular
signaling domain underlined)

SEQ ID NO: 67

mkdscitvma mallsgffff apassynldv rgarsfsppr
agrhfgyrvl qvgngvivga pgegnstgsl yqcqsgtghc
lpvtlrgsny tskylgmtla tdptdgsila cdpglsrtcd
qntylsglcy lfrqnlqgpm lqgrpgfqec ikgnvdlvfl
fdgsmslqpd efqkildfmk dvmkklsnts yqfaavqfst
syktefdfsd yvkrkdpdal lkhvkhmlll tntfgainyv
atevfreelg arpdatkvli iitdgeatds gnidaakdii
ryiigigkhf qtkesqetlh kfaskpasef vkildtfekl
kdlftelqkk iyviegtskq dltsfnmels ssgisadlsr
ghavvgavga kdwaggfldl kadlqddtfi gnepltpevr
agylgytvtw lpsrqktsll asgapryqhm grvllfqepq
ggghwsqvqt ihgtqigsyf ggelcgvdvd qdgetellli
gaplfygeqr ggrvfiyqrr qlgfeevsel qgdpgyplgr
fgeaitaltd ingdglvdva vgapleeqga vyifngrhgg
lspqpsqrie gtqvlsgiqw fgrsihgvkd legdgladva
vgaesqmivl ssrpvvdmvt lmsfspaeip vhevecsyst
snkmkegvni ticfqiksli pqfqgrlvan ltytlqldgh
rtrrrglfpg grhelrmia vttsmsctdf sfhfpvcvqd
lispinvsln fslweeegtp rdqraqgkdi ppilrpslhs
etweipfekn cgedkkcean lrvsfspars ralrltafas
lsvelslsnl eedaywvqld lhfppglsfr kvemlkphsq
ipvsceelpe esrllsrals cnvsspifka ghsvalqmmf
ntlvnsswgd svelhanvtc nnedsdlled nsattiipil
ypiniliqdq edstlyvsft pkgpkihqvk hmyqvriqps
ihdhniptle avvgvpqpps egpithqwsv qmeppvpchy
edlerlpdaa epclpgalfr cpvvfrqeil vqvigtlelv
geieassmfs lcsslsisfn sskhfhlygs naslaqvvmk
vdvvyekqml ylyvlsgigg llllllifiv lykvgffkrn
lkekmeagrg vpngipaeds eqlasgqeag dpgclkplhe
kdsesgggkd

In some embodiments, an intracellular signaling domain can be or can include a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical (or is identical) to amino acids 180 to 220 of SEQ ID NO: 68, or a fragment thereof.

(Exemplary CD11a intracellular signaling domain)
SEQ ID NO: 68
YKVGFFKRNLKEKMEAGRGVPNGIPAEDSEQLASGQEAGDPGCLKPLHEK
DSESGGGKD

An exemplary polypeptide sequence of a mouse CD11a with an exemplary intracellular signaling domain underlined is SEQ ID NO: 69. An intracellular signaling domain can be a portion of the exemplary intracellular signaling domain underlined in SEQ ID NO: 69, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 69.

(mouse CD11a with exemplary intracellular
signaling domain underlined)

SEQ ID NO: 69

msfriagprl lllglqlfak awsynldtrp tqsflaqagr
hfgyqvlqie dgvvvgapge gdntgglyhc rtssefcqpv
slhgsnhtsk ylgmtlatda akgsllacdp glsrtcdqnt
ylsglcylfp qslegpmlqn rpayqecmkg kvdlvflfdg
sqsldrkdfe kilefmkdvm rklsntsyqf aavqfstdcr
teftfldyvk qnknpdvllg svqpmflltn tfrainyvva
hvfkeesgar pdatkvlvii tdgeasdkgn isaahditry
iigigkhfvs vqkqktlhif asepveefvk ildtfeklkd
lftdlqrriy aiegtnrqdl tsfnmelsss gisadlskgh
avvgavgakd waggfldlre dlqgatfvgq epltsdvrgg
ylgytvawmt srssrpllaa gapryqhvgq vllfqapeag
grwnqtqkie gtqigsyfgg elcsvdldqd geaellliga
plffgeqrgg rvftyqrrqs lfemvselqg dpgyplgrfg
aaitaltdin gdrltdvavg apleeqgavy ifngkpggls
pqpsqriqga qvfpgirwfg rsihgvkdlg gdrladvvvg
aegrvvvlss rpvvdvvtel sfspeeipvh evecsysare
eqkhgvklka cfrikpltpq fqgrllanls ytlqldghrm
rsrglfpdgs helsgntsit pdkscldfhf hfpiciqdli
spinvslnfs lleeegtprd qkvgramqpi lrpsihtvtk
eipfekncge dkkceanltl ssparsgplr lmssaslave
wtlsnsgeda ywvrldldfp rglsfrkvem lqphsrmpvs
ceeltegssl ltktlkcnvs spifkagqev slqvmfntll
nsswedfvel ngtvhcenen sslqednsaa thipvlypvn
iltkeqenst lyisftpkgp ktqqvqhvyq vriqpsaydh
nmptlealvg vpwphsedpi tytwsvqtdp lvtchsedlk
rpsseaeqpc lpgvqfrcpi vfrreiliqv tgtvelskei
kasstlslcs slsvsfnssk hfhlygskas eaqvlvkvdl
ihekemlhvy vlsgigglvl lfliflalyk vgffkrnlke
kmeadggvpn gsppedtdpl avpgeetkdm gcleplresd
kd

In some embodiments, an intracellular signaling domain can be an intracellular signaling domain from ICAM-1 (also variously known as BB2, CD54, cluster of differentiation 54, P3.58, or intercellular adhesion molecule 1). ICAM-1 is a cell surface glycoprotein that is expressed on endothelial cells and cells of the immune system. It binds to integrins of type CD11a/CD18, or CD11b/CD18. In some embodiments, an intracellular signaling domain can be an intracellular signaling domain from a human ICAM-1 polypeptide. An exemplary polypeptide sequence of a human ICAM-1 (e.g., UniProt Protein Accession: P05362, found at URL www.uniprot.org/uniprot/P05362) with an intracellular signaling domain underlined is SEQ ID NO: 70. An intracellular signaling domain can be a portion of the exemplary intracellular signaling domain underlined in SEQ ID NO: 70, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 70.

(Human ICAM-1 polypeptide with an exemplary
intracellular signaling domain underlined)
SEQ ID NO: 70
MAPSSPRPALPALLVLLGALFPGPGNAQTSVSPSKVILPRGGSVLVTCST
SCDQPKLLGIETPLPKKELLLPGNNRKVYELSNVQEDSQPMCYSNCPDGQ
STAKTFLTVYWTPERVELAPLPSWQPVGKNLTLRCQVEGGAPRANLTVVL
LRGEKELKREPAVGEPAEVTTTVLVRRDHHGANFSCRTELDLRPQGLELF
ENTSAPYQLQTFVLPATPPQLVSPRVLEVDTQGTVVCSLDGLFPVSEAQV
HLALGDQRLNPTVTYGNDSFSAKASVSVTAEDEGTQRLTCAVILGNQSQE
TLQTVTIYSFPAPNVILTKPEVSEGTEVTVKCEAHPRAKVTLNGVPAQPL
GPRAQLLLKATPEDNGRSFSCSATLEVAGQLIHKNQTRELRVLYGPRLDE
RDCPGNWTWPENSQQTPMCQAWGNPLPELKCLKDGTFPLPIGESVTVTRD
LEGTYLCRARSTQGEVTRKVTVNVLSPRYEIVIITVVAAAVIMGTAGLST
YLYNRQRKIKKYRLQQAQKGTPMKPNTQATPP

In some embodiments, an intracellular signaling domain can be an intracellular signaling domain from a mouse ICAM-1 polypeptide. An exemplary polypeptide sequence of a mouse ICAM-1 (e.g., a polypeptide as shown in UniProt Protein Accession: P13597, found at URL www.uniprot.org/uniprot/P13597) with an exemplary intracellular signaling domain underlined is SEQ ID NO: 71. An intracellular signaling domain can be a portion of the exemplary intracellular signaling domain underlined in SEQ ID NO: 71, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 71.

(Mouse ICAM-1 polypeptide with an exemplary
intracellular signaling domain underlined)
SEQ ID NO: 71
MASTRAKPTLPLLLALVTVVIPGPGDAQVSIHPREAFLPQGGSVQVNCSS
SCKEDLSLGLETQWLKDELESGPNWKLFELSEIGEDSSPLCFENCGTVQS
SASATITVYSFPESVELRPLPAWQQVGKDLTLRCHVDGGAPRTQLSAVLL
RGEEILSRQPVGGHPKDPKEITFTVLASRGDHGANFSCRTELDLRPQGLA
LFSNVSEARSLRTFDLPATIPKLDTPDLLEVGTQQKLFCSLEGLFPASEA
RIYLELGGQMPTQESTNSSDSVSATALVEVTEEFDRTLPLRCVLELADQI
LETQRTLTVYNFSAPVLTLSQLEVSEGSQVTVKCEAHSGSKVVLLSGVEP
RPPTPQVQFTLNASSEDHKRSFFCSAALEVAGKFLFKNQTLELHVLYGPR
LDETDCLGNWTWQEGSQQTLKCQAWGNPSPKMTCRRKADGALLPIGVVKS
VKQEMNGTYVCHAFSSHGNVTRNVYLTVLYHSQNNWTIIILVPVLLVIVG
LVMAASYVYNRQRKIRIYKLQKAQEEAIKLKGQAPPP

In some embodiments, an intracellular signaling domain can be an intracellular signaling domain from NKG2C (also variously known as CD159c, NKG2-C, NKG2C, or killer cell lectin like receptor C2). NKG2C is expressed primarily in natural killer (NK) cells and acts as a receptor for the recognition of MHC class I HLA-E molecules. In some embodiments, an intracellular signaling domain can be an intracellular signaling domain from a human NKG2C polypeptide. An exemplary polypeptide sequence of a human NKG2C (e.g., UniProt Protein Accession: P26717, found at URL www.uniprot.org/uniprot/P26717) with an exemplary intracellular signaling domain underlined is SEQ ID NO: 72. An intracellular signaling domain can be a portion of the exemplary intracellular signaling domain underlined in SEQ ID NO: 72, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 72.

(Human NKG2C polypeptide with an exemplary
intracellular signaling domain underlined)
SEQ ID NO: 72
MSKQRGTFSEVSLAQDPKRQQRKPKGNKSSISGTEQEIFQVELNLQNPSL
NHQGIDKIYDCQGLLPPPEKLTAEVLGIICIVLMATVLKTIVLIPFLEQN
NSSPNTRTQKARHCGHCPEEWITYSNSCYYIGKERRTWEESLLACTSKNS
SLLSIDNEEEMKFLASILPSSWIGVFRNSSHHPWVTINGLAFKHKIKDSD
NAELNCAVLQVNRLKSAQCGSSMIYHCKHKL

In some embodiments, an intracellular signaling domain can be an intracellular signaling domain from GITR (also variously known as glucocorticoid-induced tumor necrosis factor receptor, tumor necrosis factor receptor superfamily member 18, TNFRSF18, activation-inducible TNFR family receptor, AITR, CD357, GITR-D, or TNF receptor superfamily member 18). GITR is a member of the tumor necrosis factor receptor (TNF-R) superfamily and is involved in inhibiting the suppressive activity of T-regulatory cells and extending the survival of T-effector cells. In some embodiments, an intracellular signaling domain can be an intracellular signaling domain from a human GITR polypeptide. An exemplary polypeptide sequence of a human GITR (e.g., UniProt Protein Accession: Q9Y5U5, found at URL www.uniprot.org/uniprot/Q9Y5U5) with an exemplary intracellular signaling domain underlined is SEQ ID NO: 73. An intracellular signaling domain can be a portion of the exemplary intracellular signaling domain underlined in SEQ ID NO: 73, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 73.

(Human GITR polypeptide with an exemplary
intracellular signaling domain underlined)
SEQ ID NO: 73
MAQHGAMGAFRALCGLALLCALSLGQRPTGGPGCGPGRLLLGTGTDARCC
RVHTTRCCRDYPGEECCSEWDCMCVQPEFHCGDPCCTTCRHHPCPPGQGV
QSQGKFSFGFQCIDCASGTFSGGHEGHCKPWTDCTQFGFLTVFPGNKTHN
AVCVPGSPPAEPLGWLTVVLLAVAACVLLLTSAQLGLHIWQLRSQCMWPR
ETQLLLEVPPSTEDARSCQFPEEERGERSAEEKGRLGDLWV

In some embodiments, an intracellular signaling domain can be or can include a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical (or is identical) to amino acids 180 to 220 of SEQ ID NO: 74, or a fragment thereof.

(GITR intracellular signaling domain)
SEQ ID NO: 74
QLGLHIWQLRSQCMWPRETQLLLEVPPSTEDARSCQFPEEERGERSAEEK
GRLGDLWV

In some embodiments, an intracellular signaling domain can be an intracellular signaling domain from a mouse GITR polypeptide. An exemplary polypeptide sequence of a mouse GITR (e.g., a polypeptide as shown in UniProt Protein Accession: 035714, found at URL www.uniprot.org/uniprot/035714) with an intracellular signaling domain underlined is SEQ ID NO: 75. An intracellular signaling domain can be a portion of the exemplary intracellular signaling domain underlined in SEQ ID NO: 75, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 75.

(Mouse GITR polypeptide with an exemplary
intracellular signaling domain underlined)
SEQ ID NO: 75
MGAWAMLYGVSMLCVLDLGQPSVVEEPGCGPGKVQNGSGNNTRCCSLYAP
GKEDCPKERCICVTPEYHCGDPQCKICKHYPCQPGQRVESQGDIVFGFRC
VACAMGTFSAGRDGHCRLWTNCSQFGFLTMFPGNKTHNAVCIPEPLPTEQ
YGHLTVIFLVMAACIFFLTTVQLGLHIWQLRRQHMCPRETQPFAEVQLSA
EDACSFQFPEEERGEQTEEKCHLGGRWP

In some embodiments, an intracellular signaling domain can be an intracellular signaling domain is from DAP-10 (also variously known as hematopoietic cell signal transducer, HCST, KAP10, DAP10, PIK3AP, or hematopoietic cell signal transducer). See e.g., Lanier, DAP10- and DAP12-associated receptors in innate immunity, Immunol Rev., January; 227 (1): 150-60. doi: 10.1111/j.1600-065X.2008.00720.x, 2009, incorporated by reference herein in its entirety. DAP-10 is a transmembrane signaling adaptor that contains a YxxM ITAM motif in its cytoplasmic domain. In some embodiments, an intracellular signaling domain is an intracellular signaling domain from a human DAP-10 polypeptide. An exemplary polypeptide sequence of a human DAP-10 (e.g., UniProt Protein Accession: Q9UBK5, found at URL www.uniprot.org/uniprot/Q9UBK5) an exemplary intracellular signaling domain underlined is SEQ ID NO: 76. An intracellular signaling domain can be a portion of the exemplary intracellular signaling domain underlined in SEQ ID NO: 76, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 76.

(Human DAP-10 polypeptide with an exemplary
intracellular signaling domain underlined)
SEQ ID NO: 76
MIHLGHILFLLLLPVAAAQTTPGERSSLPAFYPGTSGSCSGCGSLSLPLL
AGLVAADAVASLLIVGAVFLCARPRRSPAQEDGKVYINMPGRG

In some embodiments, an intracellular signaling domain can be or can include a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical (or is identical) to amino acids 180 to 220 of SEQ ID NO: 77, or a fragment thereof.

(DAP-10 intracellular signaling domain)

SEQ ID NO: 77

LCARPRRSPAQEDGKVYINMPGRG

In some embodiments, an intracellular signaling domain is an intracellular signaling domain from a mouse DAP-10 polypeptide. An exemplary polypeptide sequence of a mouse DAP-10 (e.g., a polypeptide as shown in UniProt Protein Accession: Q9QUJ0, found at URL www.uniprot.org/uniprot/Q9QUJ0) with an intracellular signaling domain underlined is SEQ ID NO: 78. An intracellular signaling domain can be a portion of the exemplary intracellular signaling domain underlined in SEQ ID NO: 78, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 78.

(Mouse DAP-10 polypeptide with an exemplary
intracellular signaling domain underlined)
SEQ ID NO: 78
MDPPGYLLFLLLLPVAASQTSAGSCSGCGTLSLPLLAGLVAADAVMSLLI
VGVVFVCMRPHGRPAQEDGRVYINMPGRG

In some embodiments, an intracellular signaling domain can be an intracellular signaling domain from DAP-12 (also variously known as TYROBP, DAP12, KARAP, PLOSL, and TYRO protein tyrosine kinase binding protein). See e.g., Lanier, Immunol Rev., January; 227 (1): 150-60. doi: 10.1111/j.1600-065X.2008.00720.x, 2009, incorporated by reference herein in its entirety. DAP-12 is a transmembrane signaling adaptor that contains a YxxM ITAM motif in its cytoplasmic domain. In some embodiments, an intracellular signaling domain can be an intracellular signaling domain from a human DAP-12 polypeptide. An exemplary polypeptide sequence of a human DAP-12 (e.g., UniProt Protein Accession: 043914, found at URL www.uniprot.org/uniprot/043914) with an exemplary intracellular signaling domain underlined is SEQ ID NO: 79. An intracellular signaling domain can be a portion of the exemplary intracellular signaling domain underlined in SEQ ID NO: 79, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 79.

(Human DAP-12 polypeptide with an exemplary
intracellular signaling domain underlined)
SEQ ID NO: 79
MGGLEPCSRLLLLPLLLAVSGLRPVQAQAQSDCSCSTVSPGVLAGIVMGD
LVLTVLIALAVYFLGRLVPRGRGAAEAATRKQRITETESPYQELQGQRSD
VYSDLNTQRPYYK

In some embodiments, an intracellular signaling domain can be or can include a sequence that is at least 80%, 81%, 82%, 83%, 84%, 85%, 86%, 87%, 88%, 89%, 90%, 91%, 92%, 93%, 94%, 95%, 96%, 97%, 98%, or 99% identical (or is identical) to amino acids 180 to 220 of SEQ ID NO: 80, or a fragment thereof.

(DAP-12 intracellular signaling domain)
SEQ ID NO: 80
YFLGRLVPRGRGAAEAATRKQRITETESPYQELQGQRSDVYSDLNTQRPY
YK

In some embodiments, an intracellular signaling domain can be an intracellular signaling domain from a mouse DAP-12 polypeptide. An exemplary polypeptide sequence of a mouse DAP-12 (e.g., a polypeptide as shown in UniProt Protein Accession: O54885, found at URL www.uniprot.org/uniprot/054885) with an exemplary intracellular signaling domain underlined is SEQ ID NO: 81. An intracellular signaling domain can be a portion of the exemplary intracellular signaling domain underlined in SEQ ID NO: 81, or a sequence that is at least 50% identical (e.g., at least 55%, at least 60%, at least 70%, at least 75%, at least 80%, at least 85%, at least 90%, at least 95%, or at least 99% identical) to the portion of the exemplary intracellular signaling domain underlined in SEQ ID NO: 81.

(Mouse DAP-12 polypeptide with an exemplary
intracellular signaling domain underlined)
SEQ ID NO: 81
MGALEPSWCLLFLPVLLTVGGLSPVQAQSDTFPRCDCSSVSPGVLAGIVL
GDLVLTLLIALAVYSLGRLVSRGQGTAEGTRKQHIAETESPYQELQGQRP
EVYSDLNTQRQYYR

In some embodiments, an intracellular signaling domain can be an intracellular signaling domain of B7-H3 (also called CD276). An exemplary sequence of human B7-H3 polypeptide is SEQ ID NO: 82. An exemplary intracellular signaling domain can be amino acids 488 to 534 of SEQ ID NO: 82. An intracellular signaling domain can be a sequence that is at least 50% identical (e.g., at least 55%, at least 60%, at least 70%, at least 75%, at least 80%, at least 85%, at least 90%, at least 95%, or at least 99% identical) to amino acids 488 to 534 of SEQ ID NO: 82.

(Human B7-H3 polypeptide)

SEQ ID NO: 82

mlrrrgspgm gvhvgaalga lwfcltgale vqvpedpvva
lvgtdatlcc sfspepgfsl aqlnliwqlt dtkqlvhsfa
egqdqgsaya nrtalfpdll aqgnaslrlq rvrvadegsf
tcfvsirdfg saavslqvaa pyskpsmtle pnkdlrpgdt
vtitcssyqg ypeaevfwqd gqgvpltgnv ttsqmaneqg
lfdvhsilrv vlgangtysc lvrnpvlqqd ahssvtitpq
rsptgavevq vpedpvvalv gtdatlrcsf spepgfslaq
lnliwqltdt kqlvhsfteg rdqgsayanr talfpdllaq
gnaslrlqrv rvadegsftc fvsirdfgsa avslqvaapy
skpsmtlepn kdlrpgdtvt itcssyrgyp eaevfwqdgq
gvpltgnvtt sqmaneqglf dvhsvlrvvl gangtysclv
rnpvlqqdah gsvtitgqpm tfppealwvt vglsvclial
lvalafvcwr kikqsceeen agaedqdgeg egsktalqpl
khsdskeddg qeia

In some embodiments, an intracellular signaling domain (e.g., one or more intracellular signaling domains) can be a variant polypeptide sequence that differs by at least one amino acid from an intracellular signaling domain of a wildtype protein selected from the group consisting of: Lck, FasR, TNFR-I, TNFR-II, LIGHT, ICOS, 4-1BB, CD27, OX40, CD2, CD5, CD30, CD40, CD27, CD28, CD11a, ICAM-1, B7-H3, NKG2C, GITR, DAP-10, and DAP-12. A variant polypeptide sequence, as used herein (e.g., in reference to an intracellular signaling domain) is an intracellular signaling domain having a polypeptide sequence that is at least about 80% (e.g., at least about 85%, at least about 90%, at least about 95%, at least about 96%, at least about 97%, at least about 98%, or at least about 99%) identical to the wild type polypeptide sequence of an intracellular signaling domain (e.g., of a protein selected from the group consisting of: Lck, FasR, TNFR-I, TNFR-II, LIGHT, ICOS, 4-1BB, CD27, OX40, CD2, CD5, CD30, CD40, CD27, CD28, CD11a, ICAM-1, B7-H3, NKG2C, GITR, DAP-10, and DAP-12). In some embodiments, a single-chain or a multi-chain CAR comprises two or more intracellular signaling domains, at least one of which comprises a wild type intracellular signaling domain polypeptide sequence, and at least one of which comprises a variant intracellular signaling domain polypeptide sequence. In some embodiments, a CAR comprises two or more variant intracellular signaling domains. In some embodiments, a CAR comprises a variant intracellular signaling domain comprising at least the amino acid residues that play a role in mediating signaling. For example, a CAR can comprise a variant intracellular signaling domain comprising at least the amino acid residues that play a role in interacting with various tumor necrosis factor receptor (TNF-R)-associated factor (TRAF) proteins including, but not limited to, TRAF1, TRAF2, TRAF3, and/or TRAF 5. See, e.g., Xie, TRAF molecules in cell signaling and in human diseases, J. Mol. Signal., 8 (1): 7. doi: 10.1186/1750-2187-8-7, 2013. Those of ordinary skill in the art will be aware of known structural and functional features of individual intracellular signaling domains and their partners that play a role in mediating signaling, will be able to select appropriate amino acid residues of endogenous intracellular signaling domains for use in designing variant intracellular signaling domains for use in the single-chain and multi-chain CARs described herein.

### Immunoreceptor Tyrosine-Based Activation Motifs (ITAMs)

ITAMs include a tyrosine separated from a leucine or isoleucine by any two other amino acids, and can thus be represented as, e.g., Tyr-X-X-Leu/Ile. ITAMs are typically repeated (e.g., two or more times) in the cytoplasmic tails of certain cell surface proteins of the immune system, and are typically separated by between six and eight amino acids.

In some embodiments, a single-chain chimeric antigen receptor or a multi-chain chimeric antigen receptor includes an ITAM, or portion thereof, from an endogenous mammalian (e.g., human) polypeptide, wherein endogenous mammalian (e.g., human) polypeptide is selected from the group of: CD3ξ (also referred to as CD3 zeta), CD3Δ (CD3 delta), CD3ε (CD3 epsilon), CD3γ (CD3 gamma), DAP12, FCεR1γ (Fc epsilon receptor I gamma chain), FcRy, FcRft, CD35, CD22, CD79A (antigen receptor complex-associated protein alpha chain), CD79B (antigen receptor complex-associated protein beta chain), and CD66d. The letters “CD” is the previous sentence stand for “Cluster of Differentiation.” For example, CD3 stands for “Cluster of Differentiation 3.”

Any ITAM, or portion thereof, that serves to mediate signaling in an endogenous mammalian (e.g., human) transmembrane protein suitable for use in accordance with compositions and methods disclosed herein. In some embodiments, a single-chain chimeric antigen receptor or a multi-chain chimeric antigen receptor includes an ITAM, or portion thereof, from human CD3 zeta (e.g. Accession No. P20963, e.g., an ITAM present in amino acids 52-164 of SEQ ID NO: 7, or a portion thereof; or SEQ ID NO: 83 or a portion thereof). In some embodiments, an ITAM comprises a sequence that is at least 80% (e.g., at least 82%, at least 84%, at least 86%, at least 88%, at least 90%, at least 92%, at least 94%, at least 96%, at least 98%, at least 99%, or 100% identical to: the sequence of amino acids 52-165 of SEQ ID NO: 7 (or a portion thereof), or the sequence of SEQ ID NO: 83 (or a portion thereof).

SEQ ID NO: 7
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR
(Human CD3 zeta signaling domain)
SEQ ID NO: 83
LRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
(cDNA encoding human CD3 zeta signaling
domain of SEQ ID NO: 83)
SEQ ID NO: 84
ctgagagtgaagttcagcaggagcgcagacgcccccgcgtaccagcaggg
ccagaaccagctctataacgagctcaatctaggacgaagagaggagtacg
atgttttggacaagagacgtggccgggaccctgagatggggggaaagccg
agaaggaagaaccctcaggaaggcctgtacaatgaactgcagaaagataa
gatggcggaggcctacagtgagattgggatgaaaggcgagcgccggaggg
gcaaggggcacgatggcctttaccagggtctcagtacagccaccaaggac
acctacgacgcccttcacatgcaggccctgccccctcgc

As will be appreciated by those of ordinary skill in the art, certain polypeptides have two or more isoforms that differ at least in their primary polypeptide sequence. For example, different isoforms can be generated as a result of alternative splicing. A single-chain chimeric antigen receptor or a multi-chain chimeric antigen receptor disclosed herein can include an ITAM that includes a sequence of amino acids from any isoform of an endogenous mammalian transmembrane polypeptide having an ITAM including, e.g., a mammalian (e.g., human) isoform of: CD35, CD3D, CD3E, CD3G, DAP12, FCER1G, FcRy, FcRft, CD35, CD22, CD79A, CD79B, or CD66d.

In some embodiments, an ITAM, or portion thereof, of a single-chain chimeric antigen receptor or a multi-chain chimeric antigen receptor includes a sequence of amino acids having one or more (e.g., two, three, four, or five) amino acid substitutions, deletions, or additions as compared to an ITAM of one or more of an ITAM in an endogenous mammalian (e.g., human) transmembrane protein, such as, CD35, CD3D, CD3E, CD3G, DAP12, FCERIG, FcRy, FcRft, CD35, CD22, CD79A, CD79B, or CD66d. For example, the tyrosine and leucine or isoleucine of an ITAM could be retained, while the two amino acids separating them could be replaced with different amino acids.

In some embodiments, a single-chain chimeric antigen receptor or a multi-chain chimeric antigen receptor includes an ITAM that is a chimeric ITAM having portions of an ITAM from two or more endogenous mammalian (e.g., human) transmembrane polypeptides including, without limitation, CD35, CD3D, CD3E, CD3G, DAP12, FCER1G, FcRy, FcRft, CD35, CD22, CD79A, CD79B, or CD66d (including, without limitation, a mammalian or human homolog of any of these polypeptides), such that the two or more ITAM portions together constitute a functional ITAM. In some embodiments, such a portion of a chimeric ITAM can include one or more amino acid substitutions, deletions, or additions as compared to a corresponding portion of a wild type ITAM.

In some embodiments, a single-chain chimeric antigen receptor or a multi-chain chimeric antigen receptor includes two or more ITAMs, e.g., two, three, four, or five, or more ITAMs. In some embodiments, the two or more ITAMs are identical (e.g., they have the same amino acid sequence). In some embodiments, the two or more ITAMs are not identical. For example, the ITAMs can be selected from different endogenous mammalian (e.g., human) transmembrane polypeptides including, without limitation, CD35, CD3D, CD3E, CD3G, DAP12, FCERIG, FcRy, FcRft, CD35, CD22, CD79A, CD79B (including, without limitation, a mammalian or human homolog of any of these polypeptides). In some embodiments, the two or more ITAMs can differ from each other by one or more amino acid substitutions, deletions, or additions.

### Nucleic Acids

Also provided herein are nucleic acids that encode any of the single-chain chimeric antigen receptors and multi-chain chimeric antigen receptors described herein. Also provided herein are a set of nucleic acids that encode one or more single-chain polypeptides that make up a multi-chain chimeric antigen receptor described herein (e.g., where at least one single-chain polypeptide that makes up the multi-chain chimeric antigen receptor is encoded by each nucleic acid in the set).

### Vectors

Provided herein are vectors that include any of the nucleic acids provided herein. A “vector” according to the present disclosure is a polynucleotide capable of inducing the expression of a recombinant protein (e.g., a single-chain chimeric antigen receptor or a multi-chain chimeric antigen receptor) in a mammalian cell. A vector provided herein can be, e.g., in circular or linearized form. Non-limiting examples of vectors include plasmids, SV40 vectors, adenoviral viral vectors, and adeno-associated virus (AAV) vectors. Non-limiting examples of vectors include lentiviral vectors or retroviral vectors, e.g., gamma-retroviral vectors. See, e.g., Carlens et al., Exp. Hematol. 28 (10:1137-1146, 2000; Park et al., Trends Biotechnol. 29 (11): 550-557, 2011; and Alonso-Camino et al., Mol. Ther. Nucleic Acids 2: e93, 2013. Non-limiting examples of retroviral vectors include those derived from Moloney murine leukemia virus, myeloproliferative sarcoma virus, murine embryonic stem cell virus, murine stem cell virus, spleen focus forming virus, or adeno-associated virus. Non-limiting examples of retroviral vectors are described in, e.g., U.S. Pat. Nos. 5,219,740 and 6,207,453; Miller et al., BioTechniques 7:980-990, 1989; Miller, Human Gene Therapy 1:5-14, 1990; Scarpa et al., Virology 180:849-852, 1991; Burns et al., Proc. Natl. Acad. Sci. U.S.A. 90:8033-8037, 1993; and Boris-Lawrie et al., Cur. Opin. Genet. Develop. 3:102-109, 1993. Exemplary lentiviral vectors are described in, e.g., Wang et al., J. Immunother. 35 (9): 689-701, 2003; Cooper et al., Blood 101:1637-1644, 2003; Verhoeyen et al., Methods Mol. Biol. 506:97-114, 2009; and Cavalieri et al., Blood 102 (2): 497-505, 2003.

Exemplary vectors, in which any of the nucleic acids provided herein can be inserted, are described in, e.g., Ausubel et al., Eds. “Current Protocols in Molecular Biology” Current Protocols, 1993; and Sambrook et al., Eds. “Molecular Cloning: A Laboratory Manual,” 2nd ed., Cold Spring Harbor Press, 1989.

In some embodiments, the vectors further include a promoter and/or enhancer operably linked to any of the nucleic acids described herein. Non-limiting examples of promoters include promoters from human cytomegalovirus (CMV), mouse phosphoglycerate kinase 1, polyoma adenovirus, thyroid stimulating hormone a, vimentin, simian virus 40 (SV40), tumor necrosis factor, β-globin, α-fetoprotein, γ-globin, β-interferon, γ-glutamyl transferase, human ubiquitin C (UBC), mouse mammary tumor virus (MMTV), Rous sarcoma virus, glyceraldehyde-3-phosphate dehydrogenase, β-actin, metallothionein II (MT II), amylase, human EFla, cathepsin, MI muscarinic receptor, retroviral LTR (e.g. human T-cell leukemia virus HTLV), AAV ITR, interleukin-2, collagenase, platelet-derived growth factor, adenovirus E2, stromelysin, murine MX, rat insulin, glucose regulated protein 78, human immunodeficiency virus, glucose regulated protein 94, α-2-macroglobulin, MHC class I, HSP70, proliferin, immunoglobulin light chain, T-cell receptor, HLA DQα, HLA DQβ, interleukin-2 receptor, MHC class II, prealbumin (transthyretin), elastase I, albumin, c-fos, neural cell adhesion molecule (NCAM), H2B histone, rat growth hormone, human serum amyloid (SAA), muscle creatinine kinase, troponin I (TN I), and Gibbon Ape Leukemia Virus (GALV). In some embodiments, the promoter may be an inducible promoter or a constitutive promoter. Additional examples of promoters are known in the art.

In some examples, the vectors provided herein further include a poly(A) sequence, which is operably linked and positioned 3′ to the sequence encoding the single-chain chimeric antigen receptor or any of the polypeptides that make up the multi-chain chimeric antigen receptor. Non-limiting examples of a poly(A) sequence include those derived from bovine growth hormone (Woychik et al., Proc. Natl. Acad. Sci. U.S.A. 81 (13): 3944-3948, 1984, and U.S. Pat. No. 5,122,458), mouse-β-globin, mouse-α-globin (Orkin et al., EMBO J. 4 (2): 453-456, 1985), human collagen, polyoma virus (Batt et al., Mol. Cell Biol. 15 (9): 4783-4790, 1995), the Herpes simplex virus thymidine kinase gene (HSV TK), IgG heavy chain gene polyadenylation signal (U.S. Patent Application Publication No. 2006/0040354), human growth hormone (hGH) (Szymanski et al., Mol. Therapy 15 (7): 1340-1347, 2007), SV40 poly(A) site, e.g., SV40 late and early poly(A) site (Schek et al., Mol. Cell Biol. 12 (12): 5386-5393, 1992). In some embodiments, the poly(A) sisequence includes a highly conserved upstream element (AATAAA). The this AATAAA sequence can, e.g., be substituted with other hexanucleotide sequences with homology to AATAAA which are capable of signaling polyadenylation, including, e.g., ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, and AATAAG. See, e.g., WO 06012414 A2).

A poly(A) sequence can, e.g., be a synthetic polyadenylation site. See, e.g., Levitt el al, Genes Dev. 3 (7): 1019-1025, 1989). In some examples, a poly(A) sequence can be the polyadenylation signal of soluble neuropilin-1: AAATAAAATACGAAATG (SEQ ID NO: 85). Additional examples of poly(A) sequences are known in the art. Additional examples and aspects of vectors are also known in the art.

Methods of Introducing a Nucleic Acid or Vectors into a Mammalian Cell

A variety of different methods known in the art can be used to introduce any of the nucleic acids and vectors disclosed herein into a mammalian cell (e.g., any of the mammalian cells described herein, e.g., any of the T cells (e.g., human T cells) described herein). Non-limiting examples of methods that can be used to introduce a nucleic acid or vector into a mammalian cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection. Additional methods of introducing a nucleic acid or vector into a mammalian cell are known in the art.

### Mammalian Cells

Also provided herein are mammalian cells that include any of the nucleic acids or vectors described herein. In some embodiments, the mammalian cell is previously obtained from a subject (e.g., a human subject, e.g., a human subject identified or diagnosed as having a cancer).

Non-limiting examples of mammalian cells include a T cell (e.g., a human T cell). Non limiting examples of T cells (e.g., human T cells) include, e.g., an immature thymocyte, a peripheral blood lymphocyte, a naïve T cell, a pluripotent T_{H} cell precursor, a lymphoid progenitor cell, a Treg cell, a memory T cell, a T_{H}17 cell, a T_{H}22 cell, a T_{H}9 cell, a T_{H}2 cell, a T_{H}1 cell, a T_{H}3 cell, γδ T cell, an αβ T cell, a Treg cell, and a tumor-infiltrating T cell. Additional examples of a T cell (e.g., a human T cell) include a CD8⁺ T cell, a CD4⁺ T cell, a memory T cell, a Treg cell, natural killer T cell, B cell, and a monocyte. Additional examples of mammalian cells include a mast cell, a macrophage, a neutrophil, a dendritic cell, a basophil, an eosinophil, and a natural killer cell.

### Compositions and Kits

Also provided herein are compositions (e.g., pharmaceutical compositions) that include any of the nucleic acids, vectors, sets of nucleic acids, sets of vectors, or mammalian cells described herein. For example, provided herein is a composition that includes any of the nucleic acids or sets of nucleic acids described herein, or any of the vectors or sets of vectors provided herein and a pharmaceutically acceptable solvent or carrier.

In some embodiments, a composition can be any of the mammalian cells described herein (e.g., any of the mammalian cells described herein previously obtained from a subject, e.g., a subject identified or diagnosed as having a cancer) comprising a nucleic acid encoding any of the single-chain chimeric antigen receptors or multi-chain chimeric antigen receptors described herein. In a composition including any of the mammalian cells described herein, the composition can further include a cell culture medium or a pharmaceutically acceptable buffer (e.g., phosphate-buffered saline). A composition that includes any of the mammalian cells described herein can be formulated for intravenous or intraarterial administration.

Also provided are kits that include one or more of any of the compositions described herein. In some embodiments, a kit can further include instructions for performing any of the methods described herein.

### Methods of Treating a Cancer in a Subject

Also provided herein are methods of treating a cancer in a subject (e.g., a human, a mouse, a rabbit, a rat, a horse, a dog, a monkey, or an ape) that include administering a therapeutically effective amount of any of the mammalian cells including a nucleic acid encoding any of the single-chain chimeric antigen receptors described herein or any of the multi-chain chimeric antigen receptors described herein. In some examples of these methods, the mammalian cell is a T cell (e.g., a CD8+ T cell, a CD4+ T cell, a memory T cell, a Treg cell, and a natural killer T cell). In some examples, the mammalian cell (e.g., any of the mammalian cells described herein) is a mammalian cell previously obtained from a subject (e.g., a subject that has been identified or diagnosed as having a cancer, e.g., any of the cancers described herein). Some embodiments of these methods further include obtaining the mammalian cell from the subject.

Some embodiments of these methods further include introducing a nucleic acid encoding the the single-chain chimeric antigen receptor described herein or the multi-chain chimeric antigen receptor described into a mammalian cell (e.g., any of the mammalian cells described herein or known in the art) to generate the mammalian cell that is administered to the subject.

Non-limiting examples of cancer that can be treated using any of the methods provided herein include: acute lymphoblastic leukemia, acute myeloid leukemia, adrenocortical carcinoma, Kaposi sarcoma, lymphoma, anal cancer, appendix cancer, teratoid/rhabdoid tumor, basal cell carcinoma, bile duct cancer, bladder cancer, bone cancer, brain cancer, breast cancer, bronchial tumor, carcinoid tumor, cardiac tumor, cervical cancer, chordoma, chronic lymphocytic leukemia, chronic myeloproliferative neoplasm, colon cancer, colorectal cancer, craniopharyngioma, bile duct cancer, endometrial cancer, ependymoma, esophageal cancer, esthesioneuroblastoma, Ewing sarcoma, eye cancer, fallopian tube cancer, gallbladder cancer, gastric cancer, gastrointestinal carcinoid tumor, gastrointestinal stromal tumor, germ cell tumor, hairy cell leukemia, head and neck cancer, heart cancer, liver cancer, hypopharngeal cancer, pancreatic cancer, kidney cancer, laryngeal cancer, chronic myelogenous leukemia, lip and oral cavity cancer, lung cancer, melanoma, Merkel cell carcinoma, mesothelioma, mouth cancer, oral cancer, osteosarcoma, ovarian cancer, penile cancer, pharyngeal cancer, prostate cancer, rectal cancer, salivary gland cancer, skin cancer, small intestine cancer, soft tissue sarcoma, gastric cancer, testicular cancer, throat cancer, thyroid cancer, urethral cancer, uterine cancer, vaginal cancer, and vulvar cancer.

In some embodiments of any of these methods, the methods result in a decrease in the tumor burden (e.g., tumor mass and/or volume) in a subject. For example, any of the methods described herein can result in at least about 1% to about 99% (e.g., about 1% to about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 2% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 3% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 5% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, or about 10% (inclusive); about 10% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, or about 15% (inclusive); about 15% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, or about 20% (inclusive); about 20% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, or about 25% (inclusive); about 25% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, or about 30% (inclusive); about 30% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, or about 35% (inclusive); about 35% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, or about 40% (inclusive); about 40% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, or about 45% (inclusive); about 45% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, or about 50% (inclusive); about 50% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, or about 55% (inclusive); about 55% to about 99%, about 98%, about 96%, about 94%, about 92%, about 90%, about 88%, about 86%, about 84%, about 82%, about 80%, about 78%, about 76%, about 74%, about 72%, about 70%, about 65%, or about 60% (inclusive); about 60% to about 99%, about 98%, about 96%, about 94%, about 92%, about 90%, about 88%, about 86%, about 84%, about 82%, about 80%, about 78%, about 76%, about 74%, about 72%, about 70%, or about 65% (inclusive); about 65% to about 99%, about 98%, about 96%, about 94%, about 92%, about 90%, about 88%, about 86%, about 84%, about 82%, about 80%, about 78%, about 76%, about 74%, about 72%, or about 70% (inclusive); about 70% to about 99%, about 98%, about 96%, about 94%, about 92%, about 90%, about 88%, about 86%, about 84%, about 82%, about 80%, about 78%, about 76%, about 74%, or about 72% (inclusive); about 72% to about 99%, about 98%, about 96%, about 94%, about 92%, about 90%, about 88%, about 86%, about 84%, about 82%, about 80%, about 78%, about 76%, or about 74% (inclusive); about 74% to about 99%, about 98%, about 96%, about 94%, about 92%, about 90%, about 88%, about 86%, about 84%, about 82%, about 80%, about 78%, or about 76% (inclusive); about 76% to about 99%, about 98%, about 96%, about 94%, about 92%, about 90%, about 88%, about 86%, about 84%, about 82%, about 80%, or about 78% (inclusive); about 78% to about 99%, about 98%, about 96%, about 94%, about 92%, about 90%, about 88%, about 86%, about 84%, about 82%, or about 80% (inclusive); about 80% to about 99%, about 98%, about 96%, about 94%, about 92%, about 90%, about 88%, about 86%, about 84%, or about 82% (inclusive); about 82% to about 99%, about 98%, about 96%, about 94%, about 92%, about 90%, about 88%, about 86%, or about 84% (inclusive); about 84% to about 99%, about 98%, about 96%, about 94%, about 92%, about 90%, about 88%, or about 86% (inclusive); about 86% to about 99%, about 98%, about 96%, about 94%, about 92%, about 90%, or about 88% (inclusive); about 88% to about 99%, about 98%, about 96%, about 94%, about 92%, or about 90% (inclusive); about 90% to about 99%, about 98%, about 96%, about 94%, or about 92% (inclusive); about 92% to about 99%, about 98%, about 96%, or about 94% (inclusive); about 94% to about 99%, about 98%, or about 96% (inclusive); or about 96% to about 99% or about 98% (inclusive)) reduction in the tumor burden in a subject (e.g., as compared to the tumor burden in the subject prior to treatment).

In some embodiments, the methods result in a decrease in the rate of progression of a cancer in the subject. For example, any of the methods described herein can result in at least about 1% to about 99% (e.g., about 1% to about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 2% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 3% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, about 10%, or about 5% (inclusive); about 5% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, about 15%, or about 10% (inclusive); about 10% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, about 20%, or about 15% (inclusive); about 15% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, about 25%, or about 20% (inclusive); about 20% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, about 30%, or about 25% (inclusive); about 25% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, about 35%, or about 30% (inclusive); about 30% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, about 40%, or about 35% (inclusive); about 35% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, about 45%, or about 40% (inclusive); about 40% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, about 50%, or about 45% (inclusive); about 45% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, about 55%, or about 50% (inclusive); about 50% to about 99%, about 98%, about 96%, about 94%, about 92%, about 90%, about 88%, about 86%, about 84%, about 82%, about 80%, about 78%, about 76%, about 74%, about 72%, about 70%, about 65%, about 60%, or about 55% (inclusive); about 55% to about 99%, about 98%, about 96%, about 94%, about 92%, about 90%, about 88%, about 86%, about 84%, about 82%, about 80%, about 78%, about 76%, about 74%, about 72%, about 70%, about 65%, or about 60% (inclusive); about 60% to about 99%, about 98%, about 96%, about 94%, about 92%, about 90%, about 88%, about 86%, about 84%, about 82%, about 80%, about 78%, about 76%, about 74%, about 72%, about 70%, or about 65% (inclusive); about 65% to about 99%, about 98%, about 96%, about 94%, about 92%, about 90%, about 88%, about 86%, about 84%, about 82%, about 80%, about 78%, about 76%, about 74%, about 72%, or about 70% (inclusive); about 70% to about 99%, about 98%, about 96%, about 94%, about 92%, about 90%, about 88%, about 86%, about 84%, about 82%, about 80%, about 78%, about 76%, about 74%, or about 72% (inclusive); about 72% to about 99%, about 98%, about 96%, about 94%, about 92%, about 90%, about 88%, about 86%, about 84%, about 82%, about 80%, about 78%, about 76%, or about 74% (inclusive); about 74% to about 99%, about 98%, about 96%, about 94%, about 92%, about 90%, about 88%, about 86%, about 84%, about 82%, about 80%, about 78%, or about 76% (inclusive); about 76% to about 99%, about 98%, about 96%, about 94%, about 92%, about 90%, about 88%, about 86%, about 84%, about 82%, about 80%, or about 78% (inclusive); about 78% to about 99%, about 98%, about 96%, about 94%, about 92%, about 90%, about 88%, about 86%, about 84%, about 82%, or about 80% (inclusive); about 80% to about 99%, about 98%, about 96%, about 94%, about 92%, about 90%, about 88%, about 86%, about 84%, or about 82% (inclusive); about 82% to about 99%, about 98%, about 96%, about 94%, about 92%, about 90%, about 88%, about 86%, or about 84% (inclusive); about 84% to about 99%, about 98%, about 96%, about 94%, about 92%, about 90%, about 88%, or about 86% (inclusive); about 86% to about 99%, about 98%, about 96%, about 94%, about 92%, about 90%, or about 88% (inclusive); about 88% to about 99%, about 98%, about 96%, about 94%, about 92%, or about 90% (inclusive); about 90% to about 99%, about 98%, about 96%, about 94%, or about 92% (inclusive); about 92% to about 99%, about 98%, about 96%, or about 94% (inclusive); about 94% to about 99%, about 98%, or about 96% (inclusive); or about 96% to about 99% or about 98% (inclusive)) reduction in the rate of progression of a cancer in a subject (e.g., as compared to the rate of progression of a cancer in the subject prior to treatment or in a control subject or a control population of subjects having the same cancer and administered no treatment or a different treatment).

In some embodiments of any of these methods, the methods result in an increase in the time of survival of a cancer in a subject. For example, any of the methods described herein can result in an about 1% to about 800% (e.g., about 1% to about 750%, about 700%, about 650%, about 600%, about 550%, about 500%, about 450%, about 400%, about 350%, about 300%, about 250%, about 200%, about 150%, about 100%, about 80%, about 60%, about 40%, about 20%, about 10%, or about 5% (inclusive); about 5% to about 800%, about 750%, about 700%, about 650%, about 600%, about 550%, about 500%, about 450%, about 400%, about 350%, about 300%, about 250%, about 200%, about 150%, about 100%, about 80%, about 60%, about 40%, about 20%, or about 10% (inclusive); about 10% to about 800%, about 750%, about 700%, about 650%, about 600%, about 550%, about 500%, about 450%, about 400%, about 350%, about 300%, about 250%, about 200%, about 150%, about 100%, about 80%, about 60%, about 40%, or about 20% (inclusive); about 20% to about 800%, about 750%, about 700%, about 650%, about 600%, about 550%, about 500%, about 450%, about 400%, about 350%, about 300%, about 250%, about 200%, about 150%, about 100%, about 80%, about 60%, or about 40% (inclusive); about 40% to about 800%, about 750%, about 700%, about 650%, about 600%, about 550%, about 500%, about 450%, about 400%, about 350%, about 300%, about 250%, about 200%, about 150%, about 100%, about 80%, or about 60% (inclusive); about 60% to about 800%, about 750%, about 700%, about 650%, about 600%, about 550%, about 500%, about 450%, about 400%, about 350%, about 300%, about 250%, about 200%, about 150%, about 100%, about 80% (inclusive); about 80% to about 800%, about 750%, about 700%, about 650%, about 600%, about 550%, about 500%, about 450%, about 400%, about 350%, about 300%, about 250%, about 200%, about 150%, or about 100% (inclusive); about 100% to about 800%, about 750%, about 700%, about 650%, about 600%, about 550%, about 500%, about 450%, about 400%, about 350%, about 300%, about 250%, about 200%, or about 150% (inclusive); about 150% to about 800%, about 750%, about 700%, about 650%, about 600%, about 550%, about 500%, about 450%, about 400%, about 350%, about 300%, about 250%, or about 200% (inclusive); about 200% to about 800%, about 750%, about 700%, about 650%, about 600%, about 550%, about 500%, about 450%, about 400%, about 350%, about 300%, or about 250% (inclusive); about 250% to about 800%, about 750%, about 700%, about 650%, about 600%, about 550%, about 500%, about 450%, about 400%, about 350%, or about 300% (inclusive); about 300% to about 800%, about 750%, about 700%, about 650%, about 600%, about 550%, about 500%, about 450%, about 400%, or about 350% (inclusive); about 350% to about 800%, about 750%, about 700%, about 650%, about 600%, about 550%, about 500%, about 450%, or about 400% (inclusive); about 400% to about 800%, about 750%, about 700%, about 650%, about 600%, about 550%, about 500%, or about 450% (inclusive); about 450% to about 800%, about 750%, about 700%, about 650%, about 600%, about 550%, or about 500% (inclusive); about 500% to about 800%, about 750%, about 700%, about 650%, about 600%, or about 550% (inclusive); about 550% to about 800%, about 750%, about 700%, about 650%, or about 600% (inclusive); about 600% to about 800%, about 750%, about 700%, or about 650% (inclusive); about 650% to about 800%, about 750%, or about 700% (inclusive); about 700% to about 800% or about 750% (inclusive); or about 750% to about 800% (inclusive)) increase in the time of survival of a cancer in a subject (e.g., as compared to the time of survival for a control subject or a population of control subjects having the same cancer and receiving no treatment or a different treatment).

### EXAMPLES

The invention is further described in the following examples, which do not limit the scope of the invention described in the claims.

### Example 1: Percent Lysis of Cell Lines After Co-culturing with CAR-T Cells Expressing Various CAR Constructs

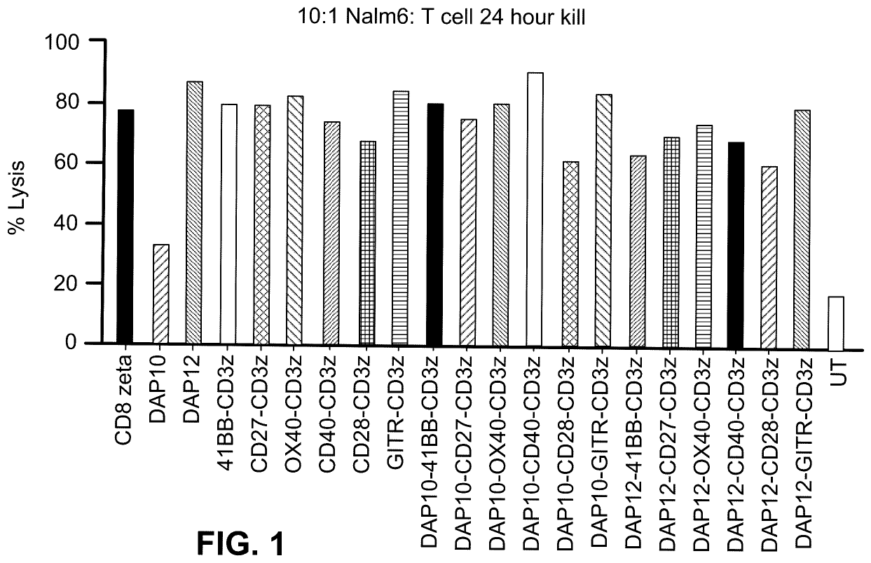

CAR-T cells were generated to express CARs that include the anti-CD19 FMC63 ScFv, the CD8 hinge region, the CD8 transmembrane region, and various co-stimulatory domains. The X axis of FIG. 1 shows the intracellular signaling domain(s) and immunoreceptor tyrosine-based activation motifs in each tested construct. The Y axis of FIG. 1 shows the percentage of target cells that were lysed in the presence of the various CAR-T cells expressing the indicated CARs.

For the various CAR constructs tested and shown in FIG. 1, the following intracellular sequences were used:

4-1BB
(SEQ ID NO: 45)
KRGRKKLLYIFKQPFMRPVQTTQEEDGCSCRFPEEEEGGCEL
GITR
(SEQ ID NO: 74)
QLGLHIWQLRSQCMWPRETQLLLEVPPSTEDARSCQFPEEERGERSAEEK
GRLGDLWV
OX40
(SEQ ID NO: 54)
ALYLLRRDQRLPPDAHKPPGGGSFRTPIQEEQADAHSTLAKI
CD27
(SEQ ID NO: 51)
QRRKYRSNKGESPVEPAEPCHYSCPREEEGSTIPIQEDYRKPEPACSP
CD40
(SEQ ID NO: 86)
KKVAKKPTNKAPHPKQEPQEINFPDDLPGSNTAAPVQETLHGCQPVTQED
GKESRISVQERQ
CD28
(SEQ ID NO: 65)
RSKRSRLLHSDYMNMTPRRPGPTRKHYQPYAPPRDFAAYRS
DAP10
(SEQ ID NO: 77)
LCARPRRSPAQEDGKVYINMPGRG
DAP12
(SEQ ID NO: 80)
YFLGRLVPRGRGAAEAATRKQRITETESPYQELQGQRSDVYSDLNTQRPY
YK
CD3z
(SEQ ID NO: 83)
LRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR

Constructs were expressed in primary T cells at roughly equivalent surface levels, cultured for fourteen days after CD3/CD28 activation and transduction, and 20,000 CAR T cells were plated at 10:1 target-to-effector cell ratios with the CD19+ luciferase transduced NALM6 fluc cell line. Nalm-6 was obtained from the ATCC, and transduced in-house. 24 hours after co-culture, NALM6_fluc lysis was quantified using a luminimeter and the percent lysis was calculated by subtracting the observed luciferase activity for each CAR-T construct from that observed in untreated cells at each concentration of 20,000 CAR-T cells. UT-Untransduced.

### Example 2: CD69 Expression of CAR-T Cells Expressing Various CAR Constructs

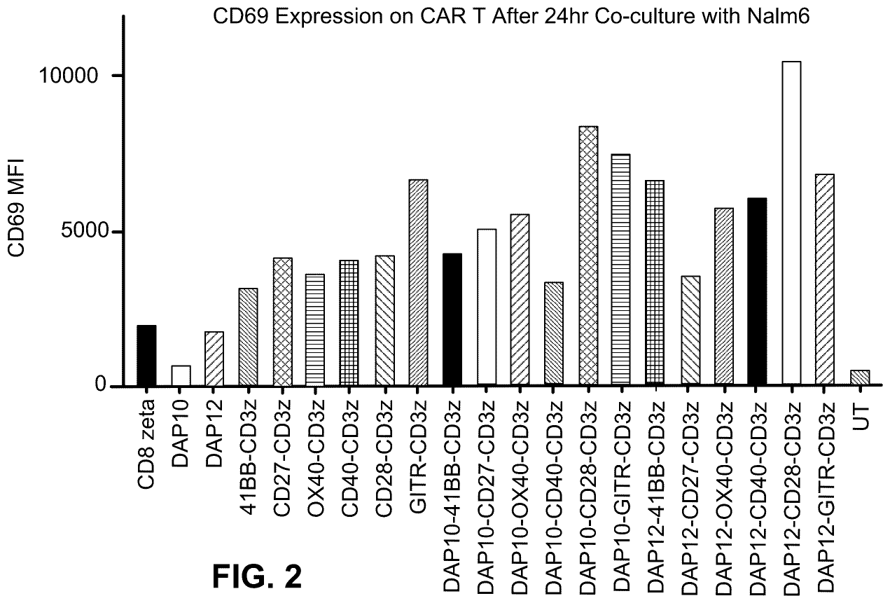

CAR-T cells were generated to express CARs that include the anti-CD19 FMC63 ScFv, the CD8 hinge region, the CD8 transmembrane region, and various co-stimulatory domains using standard molecular biology techniques. The X axis of FIG. 2 shows the intracellular signaling domain(s) and immunoreceptor tyrosine-based activation motifs in each tested construct. The sequences of the various intracellular signaling domain(s) and immunoreceptor tyrosine-based activation motifs are as described in Example 1. The Y axis of FIG. 2 shows the expression of CD69. MFI=median fluorescence intensity.

Constructs were expressed in primary T cells at roughly equivalent surface levels, cultured for fourteen days after CD3/CD28 activation and transduction, and plated at 1:1 ratios with the CD19+ cell line NALM6. 24 hours after co-culture, expression of activation marker CD69 was evaluated in the CAR-T cells by flow cytometry. UT=Untransduced

### Example 3: Cytokine Production of CAR-T Cells Expressing Various CAR Constructs

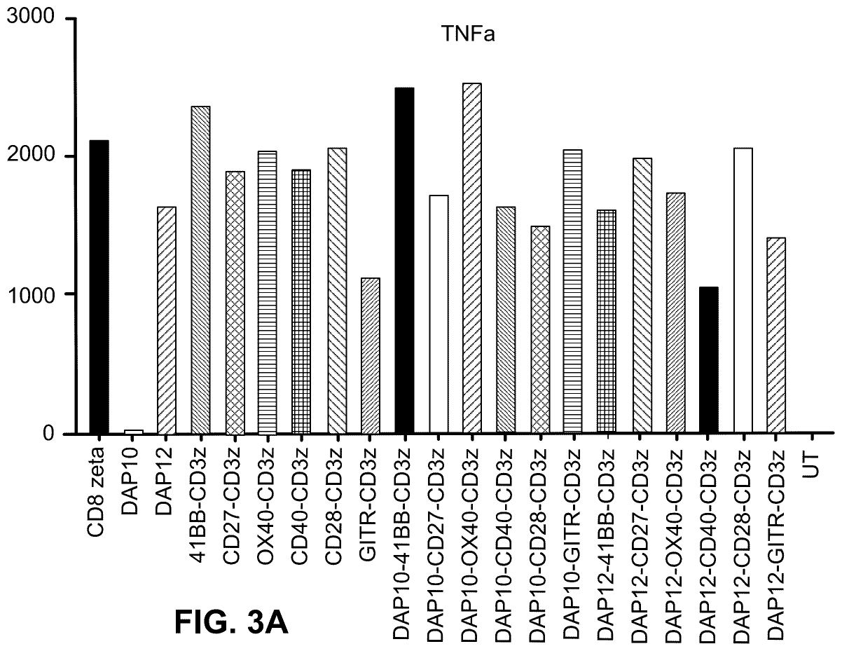

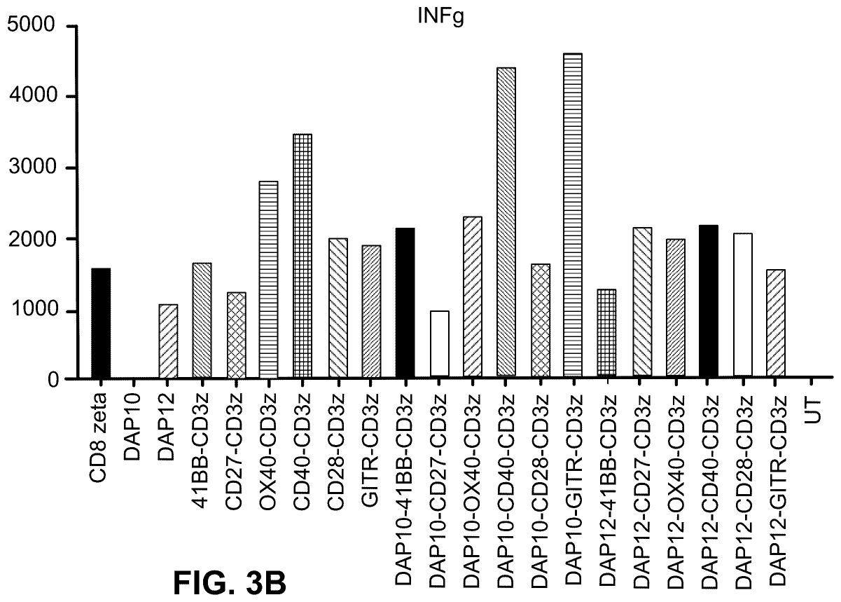

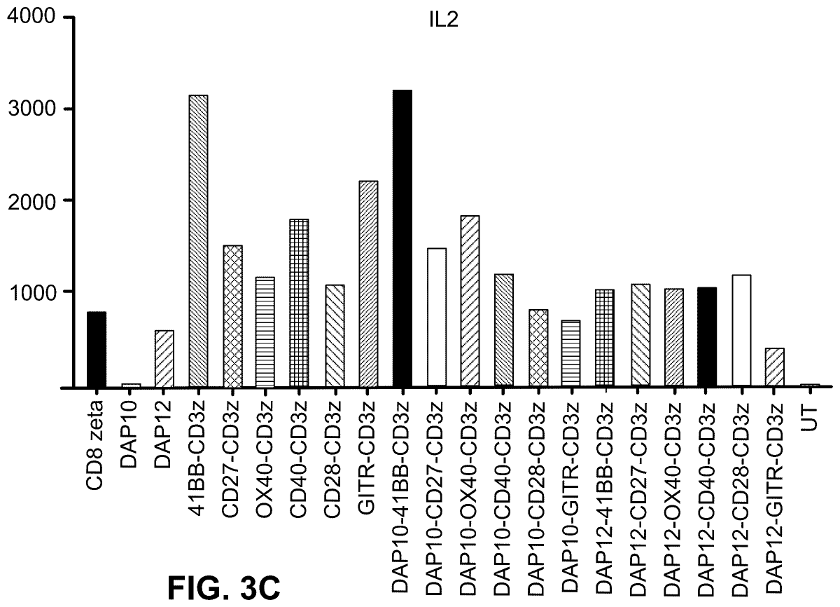

CAR-T cells were generated to express CARs that include the anti-CD19 FMC63 ScFv, the CD8 hinge region, the CD8 transmembrane region, and various co-stimulatory domains using standard molecular biology techniques. The X axes of FIGS. 3A-C show the intracellular signaling domain(s) and immunoreceptor tyrosine-based activation motifs in each tested construct. The sequences of the various intracellular signaling domain(s) and immunoreceptor tyrosine-based activation motifs are as described in Example 1. The Y axes of FIGS. 3A-C show the expression of TNF-alpha, interferon gamma, and interleukin 2 cytokines, respectively.

Constructs were expressed in primary T cells at roughly equivalent surface levels, cultured for fourteen days after CD3/CD28 activation and transduction, and plated at 1:1 ratios with the CD19+ cell line NALM6. 24 hours after co-culture, supernatant was collected from each sample and Luminex Assays Millipore/Sigma (Cat. Number HSTCMAG28SPMX21) were performed to quantitate the levels of TNF-alpha, interferon gamma, and interleukin 2 cytokines. The cytokine concentrations shown in FIGS. 3A-C are displayed in pg/mL. UT=Untransduced.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-188. (canceled)

189. A method of treating a cancer in a human subject in need thereof, the method comprising administering to the human subject a therapeutically effective amount of a human T cell, a human NKT cell, or a human NK cell expressing a nucleic acid encoding a single-chain chimeric antigen receptor, wherein the single-chain chimeric antigen receptor comprises from N-terminal to C-terminal or from C-terminal to N-terminal:
an extracellular antigen-binding domain that binds to a tumor antigen expressed on a cancer cell of the human subject;
a transmembrane domain;
a first intracellular signaling domain from human DAP-12;
a second intracellular signaling domain from human CD28; and
a CD3ξ intracellular signaling domain comprising an immunoreceptor tyrosine-based activation motif (ITAM),
wherein when the single-chain chimeric antigen receptor is expressed in a human T cell CD69 and TNFα expression is increased relative to a corresponding human T cell expressing a single-chain chimeric antigen receptor wherein the second intracellular signaling domain from human CD28 is absent or substituted with an intracellular signaling domain from human 4-1BB, CD27, OX40, CD40, or GITR.

190. The method of claim 189, wherein the extracellular antigen-binding domain is a scFv.

191. The method of claim 189, wherein the tumor antigen is selected from the group consisting of: CD19, CD20, CD22, MAGE, MUC16, WT-1, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGI, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

192. The method of claim 189, wherein the transmembrane domain comprises a human CD8α transmembrane domain.

193. The method of claim 189, wherein the single-chain polypeptide further comprises a hinge domain.

194. The method of claim 193, wherein the hinge domain comprises a human CD8α hinge domain.

195. The method of claim 189, wherein the single-chain polypeptide further comprises a hinge domain from human CD8α and wherein the transmembrane domain is from human CD8α.

196. The method of claim 189, wherein the human subject is administered the human T cell.

197. The method of claim 196, wherein the human T cell is selected from the group consisting of: a CD8⁺ T cell, a CD4⁺ T cell, a memory T cell, and a Treg cell.

198. The method of claim 190, wherein the single-chain polypeptide comprises from N-terminal to C-terminal:
the scFv;
the transmembrane domain, wherein the transmembrane domain is from human CD8α;
the first intracellular signaling domain from human DAP-12;
the second intracellular signaling domain from human CD28; and
the CD32 intracellular signaling domain.

199. The method of claim 198, wherein the single-chain polypeptide further comprises a hinge domain.

200. The method of claim 199, wherein the hinge domain comprises a human CD8α hinge domain.

201. The method of claim 190, wherein the single-chain polypeptide comprises from N-terminal to C-terminal:
the scFv;
the transmembrane domain;
the first intracellular signaling domain from human DAP-12 that comprises the sequence set forth in SEQ ID NO:80
the second intracellular signaling domain from human CD28 that comprises the sequence set forth in SEQ ID NO:65; and
the CD3ξ intracellular signaling domain that comprises the sequence set forth in SEQ ID NO: 83.

202. The method of claim 201, further comprising a hinge domain, wherein the hinge domain is positioned between the scFv and the transmembrane domain.

203. The method of claim 190, wherein the single-chain polypeptide comprises from N-terminal to C-terminal:
the scFv;
a human CD8α hinge domain that comprises the sequence set forth in SEQ ID NO:1;
a human CD8α transmembrane domain that comprises the sequence set forth in SEQ ID NO: 6;
the first intracellular signaling domain from human DAP-12 that comprises the sequence set forth in SEQ ID NO:80;
the second intracellular signaling domain from human CD28 that comprises the sequence set forth in SEQ ID NO:65; and
the CD3ξ intracellular signaling domain that comprises the sequence set forth in SEQ ID NO: 83.

204. The method of claim 189, wherein the cancer is a hematological cancer.

205. The method of claim 204, wherein the hematological cancer is a leukemia or a lymphoma.
